(19) United States
(12) Patent Application Publication
(10) Pub. No.: US 2024/0327408 A1
(43) Pub. Date: Oct. 3, 2024

(54) COMPOUNDS CONTAINING TETRAHYDRONAPHTHYRIDINONE OR TETRAHYDROPYRIDOPYRIMIDINONE SKELETON, PREPARATION METHOD THEREFOR AND PHARMACEUTICAL USE THEREOF

(71) Applicant: CHINA PHARMACEUTICAL UNIVERSITY, Jiangsu (CN)

(72) Inventors: Haiying SUN, Jiangsu (CN); Yibei XIAO, Jiangsu (CN); Yuantao FU, Jiangsu (CN); Rongliang TAN, Jiangsu (CN); Guangjun XIE, Jiangsu (CN); Jinxin JIANG, Jiangsu (CN); Yinan YUAN, Jiangsu (CN)

(73) Assignee: CHINA PHARMACEUTICAL UNIVERSITY, Jiangsu (CN)

(21) Appl. No.: 18/579,501

(22) PCT Filed: Jul. 14, 2022
(86) PCT No.: PCT/CN2022/105579

§ 371 Date: Jan. 16, 2024

(30) Foreign Application Priority Data
Jul. 16, 2021 (CN) 202110805091.6
Jul. 12, 2022 (CN) 202210814872.6

(51) Int. Cl. C07D 471/14; A61K 31/4375; A61K 31/444; A61K 31/4745; A61K 31/519; A61P 35/02; C07D 471/04; C07D 491/147; C07D 495/14; C07D 513/14

(52) U.S. Cl. CPC C07D 471/14; A61K 31/4375; A61K 31/444; A61K 31/4745; A61K 31/519; A61P 35/02; C07D 471/04; C07D 491/147; C07D 495/14; C07D 513/14

(57) ABSTRACT

The present invention is related to compounds of formula I containing a tetrahydronaphthyridinone or tetrahydropyridopyrimidinone skeleton and preparation methods therefor. It has been experimentally verified that the compounds containing a tetrahydronaphthyridinone or tetrahydropyridopyrimidinone skeleton have significant agonistic effects on caseinolytic protease P (ClpP), and can be applied in the treatment of various cancers.

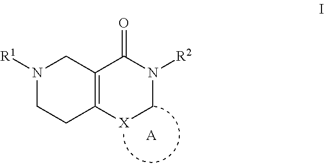

### BACKGROUND OF THE INVENTION

### 1. Technical Field

The present invention belongs to the technical field of pharmaceutical chemistry, and in particular relates to compounds containing a tetrahydronaphthyridinone or tetrahydropyridopyrimidinone skeleton, a preparation method therefor and pharmaceutical use thereof.

### 2. Background Art

Caseinolytic protease P (ClpP) is an oligomeric serine protease widely present in eukaryotic and prokaryotic cells. ClpP is a major protease in bacteria, which together with another protease LON degrade about 80% of proteins in bacteria, and its function has a key impact on bacterial infectivity. Therefore, early research on ClpP mainly focused on antibacterial drugs. ClpP is an important target for the development of antibacterial drugs against drug-resistant bacterial infections. In human cells, ClpP mainly exists in the mitochondrial matrix, can participate in the degradation of damaged or misfolded proteins in the mitochondrial matrix, and plays a key role in maintaining mitochondrial protein homeostasis. Research in recent years has found that ClpP is a unique target for anti-tumor drugs. Activation of ClpP can promote the selective degradation of ClpP substrates including a variety of respiratory chain proteins, thereby affecting the intracellular oxidative phosphorylation process and leading to the death of malignant tumor cells. ClpP has been reported to be overexpressed in a variety of cancers, including acute myeloid leukemia, breast cancer, lung cancer, liver cancer, ovarian cancer, bladder cancer, prostate cancer, uterine cancer, gastric cancer, testicular cancer, thyroid cancer, etc. (Nat. Rev. Mol. Cell Biol. 2018, 19, 109-120; Biochem. Biophys. Res. Commun. 2017, 491, 85-90; PeerJ 2020, 8, e8754), and ClpP agonists have been reported to have an inhibitory effect on the growth of various tumor cells and induce tumor cell apoptosis. For example, ADEP41 can effectively induce apoptosis in a variety of cancer cell lines, such as HeLa cervical cancer cells, U2OS osteosarcoma cells, and SH-SY5Y undifferentiated neuroblastoma cells (Cell Chem. Biol. 2018, 25, 1017-1030). In addition, the ClpP agonist ONC201 can cause tumor regression in multiple xenograft solid tumor models such as colon cancer, breast cancer, and brain tumors (Oncotarget 2016, 7, 74380-74392).

### SUMMARY OF THE INVENTION

Objective: The present invention aims to provide new ClpP agonists, i.e., compounds containing a tetrahydronaphthyridinone or tetrahydropyridopyrimidinone skeleton, which have significant agonistic effect on caseinolytic protease P (ClpP).

Technical scheme: provided are compounds of formula I or pharmaceutically acceptable salts thereof according to the present invention, wherein

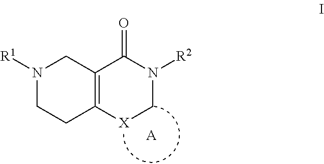

preferably, X is a carbon atom or a nitrogen atom; ring A is a substituted or unsubstituted aromatic ring or a substituted or unsubstituted aromatic heterocyclic ring;
preferably, R¹ is selected from cyclopropylmethyl, cyclobutylmethyl, cyclopentylmethyl, cyclohexylmethyl, benzyl, 2-fluorobenzyl, 2-chlorobenzyl, 2-bromobenzyl, 2-methylbenzyl, 2-ethylbenzyl, 2-methoxybenzyl, 3-fluorobenzyl, 3-chlorobenzyl, 3-bromobenzyl, 3-methylbenzyl, 3-ethylbenzyl, 3-methoxybenzyl, 3-cyanobenzyl, 4-fluorobenzyl, 4-chlorobenzyl, 4-bromobenzyl, 4-methylbenzyl, 4-ethylbenzyl, 4-methoxybenzyl, 4-trifluoromethylbenzyl, (1-methyl-1H-pyrazol-3-yl)methyl, (1-methyl-1H-pyrazol-5-yl)methyl, (1,3-dimethyl-1H-pyrazol-5-yl)methyl, pyridin-2-ylmethyl, pyridin-3-ylmethyl, pyridin-4-ylmethyl, (4-chloropyridin-2-yl)methyl, (1-methyl-1H-imidazol-2-yl)methyl, (1-methyl-1H-imidazol-4-yl)methyl, (1-methyl-1H-imidazol-5-yl)methyl, (3,5-dimethylisoxazol-4-yl)methyl, (2-methylthiazol-5-yl)methyl, benzo[1,3]dioxol-4-ylmethyl, and the like.

Preferably, R² is selected from cyclopropylmethyl, cyclobutylmethyl, cyclopentylmethyl, cyclohexylmethyl, benzyl, 2-fluorobenzyl, 2-chlorobenzyl, 2-bromobenzyl, 2-methylbenzyl, 2-ethylbenzyl, 2-methoxybenzyl, 3-fluorobenzyl, 3-chlorobenzyl, 3-bromobenzyl, 3-methylbenzyl, 3-ethylbenzyl, 3-methoxybenzyl, 4-fluorobenzyl, 4-chlorobenzyl, 4-bromobenzyl, 4-methylbenzyl, 4-ethylbenzyl, 4-methoxybenzyl, 4-nitrobenzyl, 4-methylaminobenzyl, 4-dimethylaminobenzyl, 4-trifluoromethylbenzyl, 2,4-difluorobenzyl, 2-fluoro-4-chlorobenzyl, 2-fluoro-4-methylbenzyl 2-fluoro-4-methoxybenzyl, 2-fluoro-4-trifluoromethylbenzyl, 2-fluoro-4-bromobenzyl, (1-methyl-1H-pyrazol-3-yl)methyl, (1-methyl-1H-pyrazol-5-yl)methyl, (1,3-dimethyl-1H-pyrazol-5-yl)methyl, pyridin-2-ylmethyl, pyridin-3-ylmethyl, pyridin-4-ylmethyl, (4-chloropyridin-2-yl)methyl, (1-methyl-1H-imidazol-2-yl)methyl, (1-methyl-1H-imidazol-4-yl)methyl, (1-methyl-1H-imidazol-5-yl)methyl, (3,5-dimethylisoxazol-4-yl)methyl, (2-methylthiazol-5-yl)methyl, benzo[1,3]dioxol-4-ylmethyl, benzo[1,3]dioxol-5-ylmethyl, benzo[1,4]dioxan-5-ylmethyl, benzo[1,4]dioxan-6-ylmethyl, and the like.

As a preferred technical scheme, the compounds of the present invention are shown in the following formula:

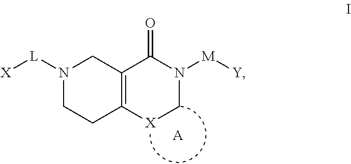

wherein: X is a carbon atom or a nitrogen atom; ring A is a benzene ring, a pyridine ring, a pyrimidine ring, an imidazole ring, a thiophene ring, a furan ring, a thiazole ring, a triazole ring, a pyrazole ring, or a pyrrole ring;
L and M are methylene and substituted methylene, respectively, and one or two hydrogens in the methylene can be substituted by C1-C6 alkyl or cycloalkyl;
X and Y are each C1-C10 alkyl or branched alkyl, C3-C10 cycloalkyl or substituted cycloalkyl, aryl or substituted aryl, or aromatic heterocyclic ring or substituted aromatic heterocyclic ring; the substituent in the group described above is halogen, alkoxy, trifluoromethyl, C1-C6 alkyl, or cycloalkyl.

Further, the preferred compounds of formula I of the present invention are as follows:
6-benzyl-3-(cyclopropylmethyl)-2,3,4,6-tetrahydropyrido[3,4-c][1,8]naphthyridine-5-(1H)-one (I-1);
6-benzyl-3-(cyclobutylmethyl)-2,3,4,6-tetrahydropyrido[3,4-c][1,8]naphthyridine-5-(1H)-one (I-2);
6-benzyl-3-(cyclopentylmethyl)-2,3,4,6-tetrahydropyrido[3,4-c][1,8]naphthyridine-5-(1H)-one (I-3);
6-benzyl-3-(cyclohexylmethyl)-2,3,4,6-tetrahydropyrido[3,4-c][1,8]naphthyridine-5-(1H)-one (I-4);
3,6-dibenzyl-2,3,4,6-tetrahydropyrido[3,4-c][1,8]naphthyridine-5-(1H)-one (I-5);
6-benzyl-3-(2-fluorobenzyl)-2,3,4,6-tetrahydropyrido[3,4-c][1,8]naphthyridine-5-(1H)-one (I-6);
6-benzyl-3-(2-chlorobenzyl)-2,3,4,6-tetrahydropyrido[3,4-c][1,8]naphthyridine-5-(1H)-one (I-7);
6-benzyl-3-(2-bromobenzyl)-2,3,4,6-tetrahydropyrido[3,4-c][1,8]naphthyridine-5-(1H)-one (I-8);
6-benzyl-3-(2-methylbenzyl)-2,3,4,6-tetrahydropyrido[3,4-c][1,8]naphthyridine-5-(1H)-one (I-9);
6-benzyl-3-(2-ethylbenzyl)-2,3,4,6-tetrahydropyrido[3,4-c][1,8]naphthyridine-5-(1H)-one (I-10);
6-benzyl-3-(2-methoxybenzyl)-2,3,4,6-tetrahydropyrido[3,4-c][1,8]naphthyridine-5-(1H)-one (I-11);
6-benzyl-3-(3-fluorobenzyl)-2,3,4,6-tetrahydropyrido[3,4-c][1,8]naphthyridine-5-(1H)-one (I-12);
6-benzyl-3-(3-chlorobenzyl)-2,3,4,6-tetrahydropyrido[3,4-c][1,8]naphthyridine-5-(1H)-one (I-13);
6-benzyl-3-(3-bromobenzyl)-2,3,4,6-tetrahydropyrido[3,4-c][1,8]naphthyridine-5-(1H)-one (I-14);
6-benzyl-3-(3-methylbenzyl)-2,3,4,6-tetrahydropyrido[3,4-c][1,8]naphthyridine-5-(1H)-one (I-15);
6-benzyl-3-(3-ethylbenzyl)-2,3,4,6-tetrahydropyrido[3,4-c][1,8]naphthyridine-5-(1H)-one (I-16);
6-benzyl-3-(3-methoxybenzyl)-2,3,4,6-tetrahydropyrido[3,4-c][1,8]naphthyridine-5-(1H)-one (I-17);
6-benzyl-3-(4-fluorobenzyl)-2,3,4,6-tetrahydropyrido[3,4-c][1,8]naphthyridine-5-(1H)-one (I-18);
6-benzyl-3-(4-chlorobenzyl)-2,3,4,6-tetrahydropyrido[3,4-c][1,8]naphthyridine-5-(1H)-one (I-19);
6-benzyl-3-(4-bromobenzyl)-2,3,4,6-tetrahydropyrido[3,4-c][1,8]naphthyridine-5-(1H)-one (I-20);
6-benzyl-3-(4-methylbenzyl)-2,3,4,6-tetrahydropyrido[3,4-c][1,8]naphthyridine-5-(1H)-one (I-21);
6-benzyl-3-(4-ethylbenzyl)-2,3,4,6-tetrahydropyrido[3,4-c][1,8]naphthyridine-5-(1H)-one (I-22);
6-benzyl-3-(4-methoxybenzyl)-2,3,4,6-tetrahydropyrido[3,4-c][1,8]naphthyridine-5-(1H)-one (I-23);
6-benzyl-3-((1-methyl-1H-pyrazol-3-yl)methyl)-2,3,4,6-tetrahydropyrido[3,4-c][1,8]naphthyridine-5-(1H)-one (I-24);
6-benzyl-3-((1-methyl-1H-pyrazol-5-yl)methyl)-2,3,4,6-tetrahydropyrido[3,4-c][1,8]naphthyridine-5-(1H)-one (I-25);
6-benzyl-3-((1,3-dimethyl-1H-pyrazol-5-yl)methyl)-2,3,4,6-tetrahydropyrido[3,4-c][1,8]naphthyridine-5-(1H)-one (I-26);
6-benzyl-3-(pyridin-2-ylmethyl)-2,3,4,6-tetrahydropyrido[3,4-c][1,8]naphthyridine-5-(1H)-one (I-27);
6-benzyl-3-(pyridin-3-ylmethyl)-2,3,4,6-tetrahydropyrido[3,4-c][1,8]naphthyridine-5-(1H)-one (I-28);
6-benzyl-3-(pyridin-4-ylmethyl)-2,3,4,6-tetrahydropyrido[3,4-c][1,8]naphthyridine-5-(1H)-one (I-29);
6-benzyl-3-((4-chloropyridin-2-yl)methyl)-2,3,4,6-tetrahydropyrido[3,4-c][1,8]naphthyridine-5-(1H)-one (I-30);
6-benzyl-3-((1-methyl-1H-imidazol-2-yl)methyl)-2,3,4,6-tetrahydropyrido[3,4-c][1,8]naphthyridine-5-(1H)-one (I-31);
6-benzyl-3-((1-methyl-1H-imidazol-4-yl)methyl)-2,3,4,6-tetrahydropyrido[3,4-c][1,8]naphthyridine-5-(1H)-one (I-32);
6-benzyl-3-((1-methyl-1H-imidazol-5-yl)methyl)-2,3,4,6-tetrahydropyrido[3,4-c][1,8]naphthyridine-5-(1H)-one (I-33);
6-benzyl-3-((3,5-dimethylisoxazol-4-yl)methyl)-2,3,4,6-tetrahydropyrido[3,4-c][1,8]naphthyridine-5-(1H)-one (I-34);
6-benzyl-3-((2-methylthiazol-5-yl)methyl)-2,3,4,6-tetrahydropyrido[3,4-c][1,8]naphthyridine-5-(1H)-one (I-35);
3-benzyl-6-(cyclopropylmethyl)-2,3,4,6-tetrahydropyrido[3,4-c][1,8]naphthyridine-5-(1H)-one (I-36);
3-benzyl-6-(cyclobutylmethyl)-2,3,4,6-tetrahydropyrido[3,4-c][1,8]naphthyridine-5-(1H)-one (I-37);
3-benzyl-6-(cyclopentylmethyl)-2,3,4,6-tetrahydropyrido[3,4-c][1,8]naphthyridine-5-(1H)-one (I-38);
3-benzyl-6-(cyclohexylmethyl)-2,3,4,6-tetrahydropyrido[3,4-c][1,8]naphthyridine-5-(1H)-one (I-39);
3-benzyl-6-(2-fluorobenzyl)-2,3,4,6-tetrahydropyrido[3,4-c][1,8]naphthyridine-5-(1H)-one (I-40);
3-benzyl-6-(2-chlorobenzyl)-2,3,4,6-tetrahydropyrido[3,4-c][1,8]naphthyridine-5-(1H)-one (I-41);
3-benzyl-6-(2-bromobenzyl)-2,3,4,6-tetrahydropyrido[3,4-c][1,8]naphthyridine-5-(1H)-one (I-42);
3-benzyl-6-(2-methylbenzyl)-2,3,4,6-tetrahydropyrido[3,4-c][1,8]naphthyridine-5-(1H)-one (I-43);
3-benzyl-6-(2-ethylbenzyl)-2,3,4,6-tetrahydropyrido[3,4-c][1,8]naphthyridine-5-(1H)-one (I-44);
3-benzyl-6-(2-methoxybenzyl)-2,3,4,6-tetrahydropyrido[3,4-c][1,8]naphthyridine-5-(1H)-one (I-45);
3-benzyl-6-(3-fluorobenzyl)-2,3,4,6-tetrahydropyrido[3,4-c][1,8]naphthyridine-5-(1H)-one (I-46);
3-benzyl-6-(3-chlorobenzyl)-2,3,4,6-tetrahydropyrido[3,4-c][1,8]naphthyridine-5-(1H)-one (I-47);
3-benzyl-6-(3-bromobenzyl)-2,3,4,6-tetrahydropyrido[3,4-c][1,8]naphthyridine-5-(1H)-one (I-48);
3-benzyl-6-(3-methylbenzyl)-2,3,4,6-tetrahydropyrido[3,4-c][1,8]naphthyridine-5-(1H)-one (I-49);
3-benzyl-6-(3-ethylbenzyl)-2,3,4,6-tetrahydropyrido[3,4-c][1,8]naphthyridine-5-(1H)-one (I-50);
3-benzyl-6-(3-methoxybenzyl)-2,3,4,6-tetrahydropyrido[3,4-c][1,8]naphthyridine-5-(1H)-one (I-51);
3-benzyl-6-(4-fluorobenzyl)-2,3,4,6-tetrahydropyrido[3,4-c][1,8]naphthyridine-5-(1H)-one (I-52);
3-benzyl-6-(4-chlorobenzyl)-2,3,4,6-tetrahydropyrido[3,4-c][1,8]naphthyridine-5-(1H)-one (I-53);
3-benzyl-6-(4-bromobenzyl)-2,3,4,6-tetrahydropyrido[3,4-c][1,8]naphthyridine-5-(1H)-one (I-54);
3-benzyl-6-(4-methylbenzyl)-2,3,4,6-tetrahydropyrido[3,4-c][1,8]naphthyridine-5-(1H)-one (I-55);
3-benzyl-6-(4-ethylbenzyl)-2,3,4,6-tetrahydropyrido[3,4-c][1,8]naphthyridine-5-(1H)-one (I-56);
3-benzyl-6-(4-methoxybenzyl)-2,3,4,6-tetrahydropyrido[3,4-c][1,8]naphthyridine-5-(1H)-one (I-57);
3-benzyl-6-((1-methyl-1H-pyrazol-3-yl)methyl)-2,3,4,6-tetrahydropyrido[3,4-c][1,8]naphthyridine-5-(1H)-one (I-58);
3-benzyl-6-((1-methyl-1H-pyrazol-5-yl)methyl)-2,3,4,6-tetrahydropyrido[3,4-c][1,8]naphthyridine-5-(1H)-one (I-59);
3-benzyl-6-((1,3-dimethyl-1H-pyrazol-5-yl)methyl)-2,3,4,6-tetrahydropyrido[3,4-c][1,8]naphthyridine-5-(1H)-one (I-60);
3-benzyl-6-(pyridin-2-ylmethyl)-2,3,4,6-tetrahydropyrido[3,4-c][1,8]naphthyridine-5-(1H)-one (I-61);
3-benzyl-6-(pyridin-3-ylmethyl)-2,3,4,6-tetrahydropyrido[3,4-c][1,8]naphthyridine-5-(1H)-one (I-62);
3-benzyl-6-(pyridin-4-ylmethyl)-2,3,4,6-tetrahydropyrido[3,4-c][1,8]naphthyridine-5-(1H)-one (I-63);
3-benzyl-6-((4-chloropyridin-2-yl)methyl)-2,3,4,6-tetrahydropyrido[3,4-c][1,8]naphthyridine-5-(1H)-one (I-64);
3-benzyl-6-((1-methyl-1H-imidazol-2-yl)methyl)-2,3,4,6-tetrahydropyrido[3,4-c][1,8]naphthyridine-5-(1H)-one (I-65);
3-benzyl-6-((1-methyl-1H-imidazol-4-yl)methyl)-2,3,4,6-tetrahydropyrido[3,4-c][1,8]naphthyridine-5-(1H)-one (I-66);
3-benzyl-6-((1-methyl-1H-imidazol-5-yl)methyl)-2,3,4,6-tetrahydropyrido[3,4-c][1,8]naphthyridine-5-(1H)-one (I-67);
3-benzyl-6-((3,5-dimethylisoxazol-4-yl)methyl)-2,3,4,6-tetrahydropyrido[3,4-c][1,8]naphthyridine-5-(1H)-one (I-68);
3-benzyl-6-((2-methylthiazol-5-yl)methyl)-2,3,4,6-tetrahydropyrido[3,4-c][1,8]naphthyridine-5-(1H)-one (I-69);
3-benzyl-6-(4-(trifluoromethyl)benzyl)-2,3,4,6-tetrahydropyrido[3,4-c][1,8]naphthyridine-5-(1H)-one (I-70);
3-(3-fluorobenzyl)-6-(4-fluorobenzyl)-2,3,4,6-tetrahydropyrido[3,4-c][1,8]naphthyridine-5-(1H)-one (I-71);
3-(3-chlorobenzyl)-6-(4-fluorobenzyl)-2,3,4,6-tetrahydropyrido[3,4-c][1,8]naphthyridine-5-(1H)-one (I-72);
3-(3-bromobenzyl)-6-(4-fluorobenzyl)-2,3,4,6-tetrahydropyrido[3,4-c][1,8]naphthyridine-5-(1H)-one (I-73);
3-(3-fluorobenzyl)-6-(4-chlorobenzyl)-2,3,4,6-tetrahydropyrido[3,4-c][1,8]naphthyridine-5-(1H)-one (I-74);
3-(3-chlorobenzyl)-6-(4-chlorobenzyl)-2,3,4,6-tetrahydropyrido[3,4-c][1,8]naphthyridine-5-(1H)-one (I-75);
3-(3-bromobenzyl)-6-(4-chlorobenzyl)-2,3,4,6-tetrahydropyrido[3,4-c][1,8]naphthyridine-5-(1H)-one (I-76);
3-(3-fluorobenzyl)-6-(4-bromobenzyl)-2,3,4,6-tetrahydropyrido[3,4-c][1,8]naphthyridine-5-(1H)-one (I-77);
3-(3-chlorobenzyl)-6-(4-bromobenzyl)-2,3,4,6-tetrahydropyrido[3,4-c][1,8]naphthyridine-5-(1H)-one (I-78);
3-(3-bromobenzyl)-6-(4-bromobenzyl)-2,3,4,6-tetrahydropyrido[3,4-c][1,8]naphthyridine-5-(1H)-one (I-79);
3-benzyl-6-(4-(trifluoromethyl)benzyl)-2,3,4,6-tetrahydrobenzo[c][2,7]naphthyridine-5-(1H)-one (I-80);
8-benzyl-5-(4-(trifluoromethyl)benzyl)-7,8,9,10-tetrahydropyrimidinyl[4,5-c][2,7]naphthyridine-6-(5H)-one (I-81);
6-benzyl-3-(3-cyanobenzyl)-2,3,4,6-tetrahydropyrido[3,4-c][1,8]naphthyridine-5-(1H)-one (I-82);
6-(4-trifluoromethylbenzyl)-3-(3-cyanobenzyl)-2,3,4,6-tetrahydropyrido[3,4-c][1,8]naphthyridine-5-(1H)-one (I-83);
6-(4-fluorobenzyl)-3-(3-cyanobenzyl)-2,3,4,6-tetrahydropyrido[3,4-c][1,8]naphthyridine-5-(1H)-one (I-84);
6-(4-chlorobenzyl)-3-(3-cyanobenzyl)-2,3,4,6-tetrahydropyrido[3,4-c][1,8]naphthyridine-5-(1H)-one (I-85);
6-(4-bromobenzyl)-3-(3-cyanobenzyl)-2,3,4,6-tetrahydropyrido[3,4-c][1,8]naphthyridine-5-(1H)-one (I-86);
6-(4-methoxybenzyl)-3-(3-cyanobenzyl)-2,3,4,6-tetrahydropyrido[3,4-c][1,8]naphthyridine-5-(1H)-one (I-87);
6-(4-nitrobenzyl)-3-(3-cyanobenzyl)-2,3,4,6-tetrahydropyrido[3,4-c][1,8]naphthyridine-5-(1H)-one (I-88);
6-(4-methylaminobenzyl)-3-(3-cyanobenzyl)-2,3,4,6-tetrahydropyrido[3,4-c][1,8]naphthyridine-5-(1H)-one (I-89);
6-(4-dimethylaminobenzyl)-3-(3-cyanobenzyl)-2,3,4,6-tetrahydropyrido[3,4-c][1,8]naphthyridine-5-(1H)-one (I-90);
6-(4-methylbenzyl)-3-(3-cyanobenzyl)-2,3,4,6-tetrahydropyrido[3,4-c][1,8]naphthyridine-5-(1H)-one (I-91);
6-(4-methylbenzyl)-3-(3-chlorobenzyl)-2,3,4,6-tetrahydropyrido[3,4-c][1,8]naphthyridine-5-(1H)-one (I-92);
6-(4-methylbenzyl)-3-(3-fluorobenzyl)-2,3,4,6-tetrahydropyrido[3,4-c][1,8]naphthyridine-5-(1H)-one (I-93);
6-(4-ethylbenzyl)-3-(3-cyanobenzyl)-2,3,4,6-tetrahydropyrido[3,4-c][1,8]naphthyridine-5-(1H)-one (I-94);
6-(4-ethylbenzyl)-3-(3-fluorobenzyl)-2,3,4,6-tetrahydropyrido[3,4-c][1,8]naphthyridine-5-(1H)-one (I-95);
6-(4-ethylbenzyl)-3-(3-chlorobenzyl)-2,3,4,6-tetrahydropyrido[3,4-c][1,8]naphthyridine-5-(1H)-one (I-96);
3-(3-chlorobenzyl)-6-((2,3-dihydro[1,4]benzodioxin-6-yl)methyl)-2,3,4,6-tetrahydropyrido[3,4-c][1,8]naphthyridine-5(1H)-one (I-97);
6-([1,3]benzodioxol-5-ylmethyl)-3-(3-cyanobenzyl)-2,3,4,6-tetrahydropyrido[3,4-c][1,8]naphthyridine-5-(1H)-one (I-98);
6-(4-methoxybenzyl)-3-(3-fluorobenzyl)-2,3,4,6-tetrahydropyrido[3,4-c][1,8]naphthyridine-5-(1H)-one (I-99);
6-(4-methoxybenzyl)-3-(3-chlorobenzyl)-2,3,4,6-tetrahydropyrido[3,4-c][1,8]naphthyridine-5-(1H)-one (I-100);
7-benzyl-4-(4-trifluoromethylbenzyl)-6,7,8,9-tetrahydrofuro[3,4-C][2,7]naphthyridine-5(4H)-one (I-101);
7-benzyl-4-(4-trifluoromethylbenzyl)-6,7,8,9-tetrahydrothieno[3,4-C][2,7]naphthyridine-5(4H)-one (I-102);
7-benzyl-4-(4-trifluoromethylbenzyl)-6,7,8,9-tetrahydrothieno[3,2-C][2,7]naphthyridine-5(4H)-one (I-103);
7-benzyl-4-(4-trifluoromethylbenzyl)-6,7,8,9-tetrahydrothieno[2,3-C][2,7]naphthyridine-5(4H)-one (I-104);
7-benzyl-4-(4-trifluoromethylbenzyl)-6,7,8,9-tetrahydrothiazolo[4,5-C][2,7]naphthyridine-5(4H)-one (I-105);
7-benzyl-4-(2-methylbenzyl)-6,7,8,9-tetrahydroimidazo[1,2-a]pyrido[3,4-e]pyrimidine-5(4)-one (I-106);
4,7-dibenzyl-6,7,8,9-tetrahydroimidazo[1,2-a]pyrido[3,4-e]pyrimidine-5(4H)-one (I-107);
7-(3-fluorobenzyl)-4-benzyl-6,7,8,9-tetrahydroimidazo[1,2-a]pyrido[3,4-e]pyrimidine-5(4H)-one (I-108);
7-(3-chlorobenzyl)-4-benzyl-6,7,8,9-tetrahydroimidazo[1,2-a]pyrido[3,4-e]pyrimidine-5(4H)-one (I-109);
7-(3-methylbenzyl)-4-benzyl-6,7,8,9-tetrahydroimidazo[1,2-a]pyrido[3,4-e]pyrimidine-5(4H)-one (I-110);
7-(3-cyanobenzyl)-4-benzyl-6,7,8,9-tetrahydroimidazo[1,2-a]pyrido[3,4-e]pyrimidine-5(4H)-one (I-111);
7-benzyl-4-(4-fluorobenzyl)-6,7,8,9-tetrahydroimidazo[1,2-a]pyrido[3,4-e]pyrimidine-5(4H)-one (I-112);
7-(3-fluorobenzyl)-4-(4-fluorobenzyl)-6,7,8,9-tetrahydroimidazo[1,2-a]pyrido[3,4-e]pyrimidine-5(4H)-one (I-113);
7-(3-chlorobenzyl)-4-(4-fluorobenzyl)-6,7,8,9-tetrahydroimidazo[1,2-a]pyrido[3,4-e]pyrimidine-5(4H)-one (I-114);
7-(3-methylbenzyl)-4-(4-fluorobenzyl)-6,7,8,9-tetrahydroimidazo[1,2-a]pyrido[3,4-e]pyrimidine-5(4H)-one (I-115);
7-(3-cyanobenzyl)-4-(4-fluorobenzyl)-6,7,8,9-tetrahydroimidazo[1,2-a]pyrido[3,4-e]pyrimidine-5(4H)-one (I-116);
7-benzyl-4-(4-chlorobenzyl)-6,7,8,9-tetrahydroimidazo[1,2-a]pyrido[3,4-e]pyrimidine-5(4H)-one (I-117);
7-(3-fluorobenzyl)-4-(4-chlorobenzyl)-6,7,8,9-tetrahydroimidazo[1,2-a]pyrido[3,4-e]pyrimidine-5(4H)-one (I-118);
7-(3-chlorobenzyl)-4-(4-chlorobenzyl)-6,7,8,9-tetrahydroimidazo[1,2-a]pyrido[3,4-e]pyrimidine-5(4H)-one (I-119);
7-(3-methylbenzyl)-4-(4-chlorobenzyl)-6,7,8,9-tetrahydroimidazo[1,2-a]pyrido[3,4-e]pyrimidine-5(4H)-one (I-120);
7-(3-cyanobenzyl)-4-(4-chlorobenzyl)-6,7,8,9-tetrahydroimidazo[1,2-a]pyrido[3,4-e]pyrimidine-5(4H)-one (I-121);
7-benzyl-4-(2,4-difluorobenzyl)-6,7,8,9-tetrahydroimidazo[1,2-a]pyrido[3,4-e]pyrimidine-5(4H)-one (I-122);
7-(3-fluorobenzyl)-4-(2,4-difluorobenzyl)-6,7,8,9-tetrahydroimidazo[1,2-a]pyrido[3,4-e]pyrimidine-5(4H)-one (I-123);
7-(3-chlorobenzyl)-4-(2,4-difluorobenzyl)-6,7,8,9-tetrahydroimidazo[1,2-a]pyrido[3,4-e]pyrimidine-5(4H)-one (I-124);
7-(3-methylbenzyl)-4-(2,4-difluorobenzyl)-6,7,8,9-tetrahydroimidazo[1,2-a]pyrido[3,4-e]pyrimidine-5(4H)-one (I-125);
7-(3-cyanobenzyl)-4-(2,4-difluorobenzyl)-6,7,8,9-tetrahydroimidazo[1,2-a]pyrido[3,4-e]pyrimidine-5(4H)-one (I-126);
7-benzyl-4-(4-trifluoromethylbenzyl)-6,7,8,9-tetrahydroimidazo[1,2-a]pyrido[3,4-e]pyrimidine-5(4H)-one (I-127);
7-(3-fluorobenzyl)-4-(4-trifluoromethylbenzyl)-6,7,8,9-tetrahydroimidazo[1,2-a]pyrido[3,4-e]pyrimidine-5(4H)-one (I-128);
7-(3-chlorobenzyl)-4-(4-trifluoromethylbenzyl)-6,7,8,9-tetrahydroimidazo[1,2-a]pyrido[3,4-e]pyrimidine-5(4H)-one (I-129);
7-(3-methylbenzyl)-4-(4-trifluoromethylbenzyl)-6,7,8,9-tetrahydroimidazo[1,2-a]pyrido[3,4-e]pyrimidine-5(4H)-one (I-130);
7-(3-cyanobenzyl)-4-(4-trifluoromethylbenzyl)-6,7,8,9-tetrahydroimidazo[1,2-a]pyrido[3,4-e]pyrimidine-5(4H)-one (I-131);
7-benzyl-4-(4-methylbenzyl)-6,7,8,9-tetrahydroimidazo[1,2-a]pyrido[3,4-e]pyrimidine-5(4H)-one (I-132);
7-(3-fluorobenzyl)-4-(4-methylbenzyl)-6,7,8,9-tetrahydroimidazo[1,2-a]pyrido[3,4-e]pyrimidine-5(4H)-one (I-133);
7-(3-chlorobenzyl)-4-(4-methylbenzyl)-6,7,8,9-tetrahydroimidazo[1,2-a]pyrido[3,4-e]pyrimidine-5(4H)-one (I-134);
7-(3-methylbenzyl)-4-(4-methylbenzyl)-6,7,8,9-tetrahydroimidazo[1,2-a]pyrido[3,4-e]pyrimidine-5(4H)-one (I-135);
7-(3-cyanobenzyl)-4-(4-methylbenzyl)-6,7,8,9-tetrahydroimidazo[1,2-a]pyrido[3,4-e]pyrimidine-5(4H)-one (I-136);
7-benzyl-4-(4-ethylbenzyl)-6,7,8,9-tetrahydroimidazo[1,2-a]pyrido[3,4-e]pyrimidine-5(4H)-one (I-137);
7-(3-fluorobenzyl)-4-(4-ethylbenzyl)-6,7,8,9-tetrahydroimidazo[1,2-a]pyrido[3,4-e]pyrimidine-5(4H)-one (I-138);
7-(3-chlorobenzyl)-4-(4-ethylbenzyl)-6,7,8,9-tetrahydroimidazo[1,2-a]pyrido[3,4-e]pyrimidine-5(4H)-one (I-139);
7-(3-methylbenzyl)-4-(4-ethylbenzyl)-6,7,8,9-tetrahydroimidazo[1,2-a]pyrido[3,4-e]pyrimidine-5(4H)-one (I-140);
7-(3-cyanobenzyl)-4-(4-ethylbenzyl)-6,7,8,9-tetrahydroimidazo[1,2-a]pyrido[3,4-e]pyrimidine-5(4H)-one (I-141);
7-benzyl-4-(4-methoxybenzyl)-6,7,8,9-tetrahydroimidazo[1,2-a]pyrido[3,4-e]pyrimidine-5(4H)-one (I-142);
7-(3-fluorobenzyl)-4-(4-methoxybenzyl)-6,7,8,9-tetrahydroimidazo[1,2-a]pyrido[3,4-e]pyrimidine-5(4H)-one (I-143);
7-(3-chlorobenzyl)-4-(4-methoxybenzyl)-6,7,8,9-tetrahydroimidazo[1,2-a]pyrido[3,4-e]pyrimidine-5(4H)-one (I-144);
7-(3-methylbenzyl)-4-(4-methoxybenzyl)-6,7,8,9-tetrahydroimidazo[1,2-a]pyrido[3,4-e]pyrimidine-5(4H)-one (I-145);
7-(3-cyanobenzyl)-4-(4-methoxybenzyl)-6,7,8,9-tetrahydroimidazo[1,2-a]pyrido[3,4-e]pyrimidine-5(4H)-one (I-146);
7-(3-chlorobenzyl)-4-([1,3]benzodioxol-5-ylmethyl)-6,7,8,9-tetrahydroimidazo[1,2-a]pyrido[3,4-e]pyrimidine-5(4H)-one (I-147);
7-(3-chlorobenzyl)-4-((2,3-dihydro[1,4]benzodioxin-6-yl)methyl)-6,7,8,9-tetrahydroimidazo[1,2-a]pyrido[3,4-e]pyrimidine-5(4H)-one (I-148);
4-(4-trifluoromethylbenzyl)-7-benzyl-6,7,8,9-tetrahydropyrido[3,4-e][1,2,4]triazolo[1,5-a]pyrimidine-5(4H)-one (I-149);
4-(4-trifluoromethylbenzyl)-7-(3-chlorobenzyl)-6,7,8,9-tetrahydropyrido[3,4-e][1,2,4]triazolo[1,5-a]pyrimidine-5(4H)-one (I-150);
4-(4-trifluoromethylbenzyl)-7-(3-cyanobenzyl)-6,7,8,9-tetrahydropyrido[3,4-e][1,2,4]triazolo[1,5-a]pyrimidine-5(4H)-one (I-151);
4-(4-chlorobenzyl)-7-benzyl-6,7,8,9-tetrahydropyrido[3,4-e][1,2,4]triazolo[1,5-a]pyrimidine-5(4H)-one (I-152);
4-(4-chlorobenzyl)-7-(3-fluorobenzyl)-6,7,8,9-tetrahydropyrido[3,4-e][1,2,4]triazolo[1,5-a]pyrimidine-5(4H)-one (I-153);
4-(4-chlorobenzyl)-7-(3-chlorobenzyl)-6,7,8,9-tetrahydropyrido[3,4-e][1,2,4]triazolo[1,5-a]pyrimidine-5(4H)-one (I-154);
4-(4-chlorobenzyl)-7-(3-cyanobenzyl)-6,7,8,9-tetrahydropyrido[3,4-e][1,2,4]triazolo[1,5-a]pyrimidine-5(4H)-one (I-155);
4-benzyl-7-(3-chlorobenzyl)-6,7,8,9-tetrahydropyrazolo[1,5-a]pyrido[3,4-e]pyrimidine-5(4H)-one (I-156);
4-benzyl-7-(3-cyanobenzyl)-6,7,8,9-tetrahydropyrazolo[1,5-a]pyrido[3,4-e]pyrimidine-5(4H)-one (I-157);
4-benzyl-7-(3-methylbenzyl)-6,7,8,9-tetrahydropyrazolo[1,5-a]pyrido[3,4-e]pyrimidine-5(4H)-one (I-158);
4-(4-trifluoromethylbenzyl)-7-benzyl-6,7,8,9-tetrahydropyrazolo[1,5-a]pyrido[3,4-e]pyrimidine-5(4H)-one (I-159);
4-(4-trifluoromethylbenzyl)-7-(3-cyanobenzyl)-6,7,8,9-tetrahydropyrazolo[1,5-a]pyrido[3,4-e]pyrimidine-5(4H)-one (I-160);
4-(4-chlorobenzyl)-7-benzyl-6,7,8,9-tetrahydropyrazolo[1,5-a]pyrido[3,4-e]pyrimidine-5(4H)-one (I-161);
4-(4-chlorobenzyl)-7-(3-chlorobenzyl)-6,7,8,9-tetrahydropyrazolo[1,5-a]pyrido[3,4-e]pyrimidine-5(4H)-one (I-162);
4-(4-chlorobenzyl)-7-(3-cyanobenzyl)-6,7,8,9-tetrahydropyrazolo[1,5-a]pyrido[3,4-e]pyrimidine-5(4H)-one (I-163);
7-benzyl-4-(4-trifluoromethylbenzyl)-6,7,8,9-tetrahydroimidazo[1,5-a]pyrido[3,4-e]pyrimidine-5(4H)-one (I-164);
7-(3-cyanobenzyl)-4-(4-trifluoromethylbenzyl)-6,7,8,9-tetrahydroimidazo[1,5-a]pyrido[3,4-e]pyrimidine-5(4H)-one (I-165);
7-(3-chlorobenzyl)-4-(4-chlorobenzyl)-6,7,8,9-tetrahydroimidazo[1,5-a]pyrido[3,4-e]pyrimidine-5(4H)-one (I-166);
7-(3-cyanobenzyl)-4-(4-chlorobenzyl)-6,7,8,9-tetrahydroimidazo[1,5-a]pyrido[3,4-e]pyrimidine-5(4H)-one (I-167);
3-benzyl-6-(4-trifluoromethylbenzyl)-1,2,3,4-tetrahydropyrido[3,4-e]pyrrolo[1,2-a]pyrimidine-5(6H)-one (I-168); and
3-(3-chlorobenzyl)-6-(4-chlorobenzyl)-1,2,3,4-tetrahydropyrido[3,4-e]pyrrolo[1,2-a]pyrimidine-5(6H)-one (I-169).

Provided is a preparation method for the compounds represented by formula I described above, wherein when ring A is of a structure represented by general formula II, a synthetic route comprises

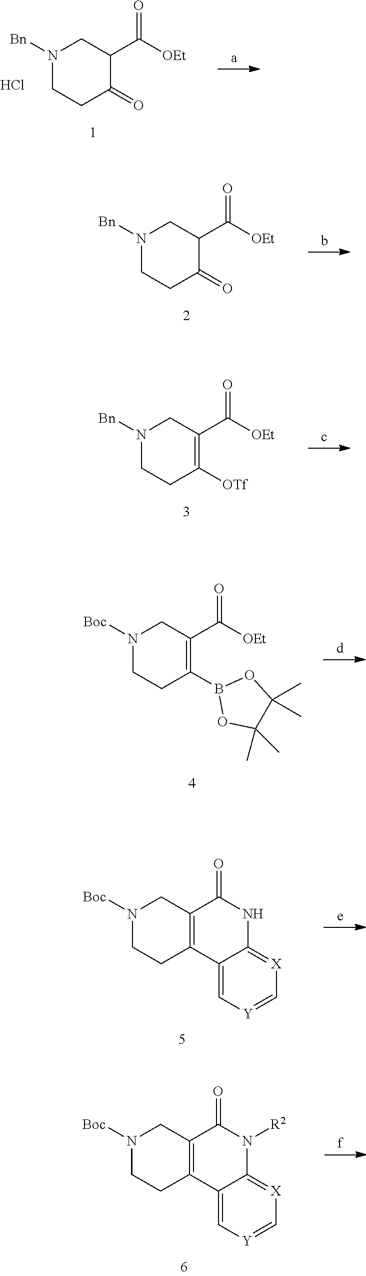

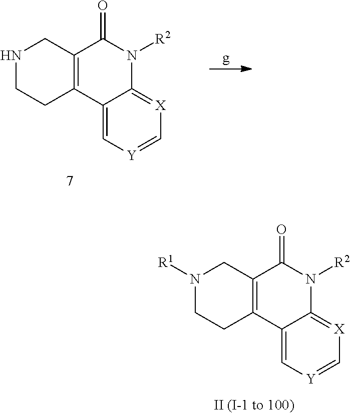

the following steps:
(1) step a: after hydrogenation to remove a benzyl group, reacting compound 1 with di-tert-butyl dicarbonate to prepare compound 2;
(2) step b: reacting compound 2 with trifluoromethanesulfonic anhydride to prepare compound 3;
(3) step c: reacting compound 3 with bis(pinacolato)diboron to prepare compound 4;
(4) step d: reacting compound 4 with iodo- and amino-disubstituted pyridine, benzene or pyrimidine to prepare compound 5;
(5) step e: reacting compound 5 with different halogenated hydrocarbons to prepare compound 6;
(6) step f: deprotecting compound 6 with trifluoroacetic acid from a tert-butoxycarbonyl group to prepare compound 7; and
(7) step g: reacting compound 7 with different halogenated hydrocarbons to prepare compounds represented by general formula II (I-1 to I-100);
compound 4 can be reacted with other heterocyclic aromatic amines substituted with ortho-iodo or ortho-bromo according to the method described above to synthesize compounds of general formula I (I-101 to I-105) in which ring A is other aromatic heterocyclic rings, including furan, thiophene, thiazole, and the like.

When ring A in general formula I is an imidazole ring in general formula III, a synthetic route comprises

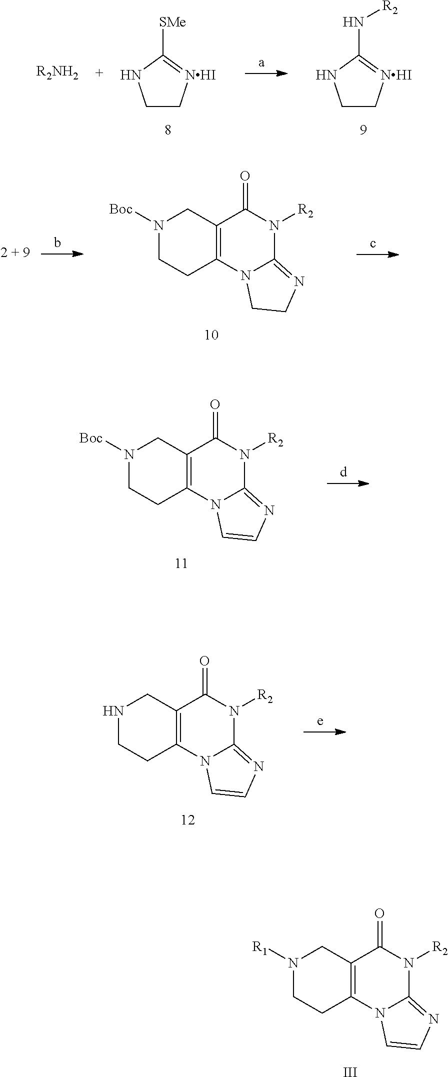

the following steps:
(1) step a: reacting compound 8 with different amines to prepare compound 9;
(2) step b: reacting compound 2 with compound 9 to prepare compound 10;
(3) step c: oxidizing a dihydroimidazole ring in compound 10 into an imidazole ring to prepare compound 11;
(4) step d: deprotecting compound 11 with trifluoroacetic acid from a tert-butoxycarbonyl group to prepare compound 12; and
(5) step e: reacting compound 12 with different halogenated hydrocarbons to prepare compounds I-106 to I-141 represented by general formula III.

When ring A in the general formula I is an imidazole ring, a triazole ring, a pyrazole ring or a pyrrole ring in general formula IV, and X, Y and Z are each a nitrogen atom or a methine group, a synthetic route comprises

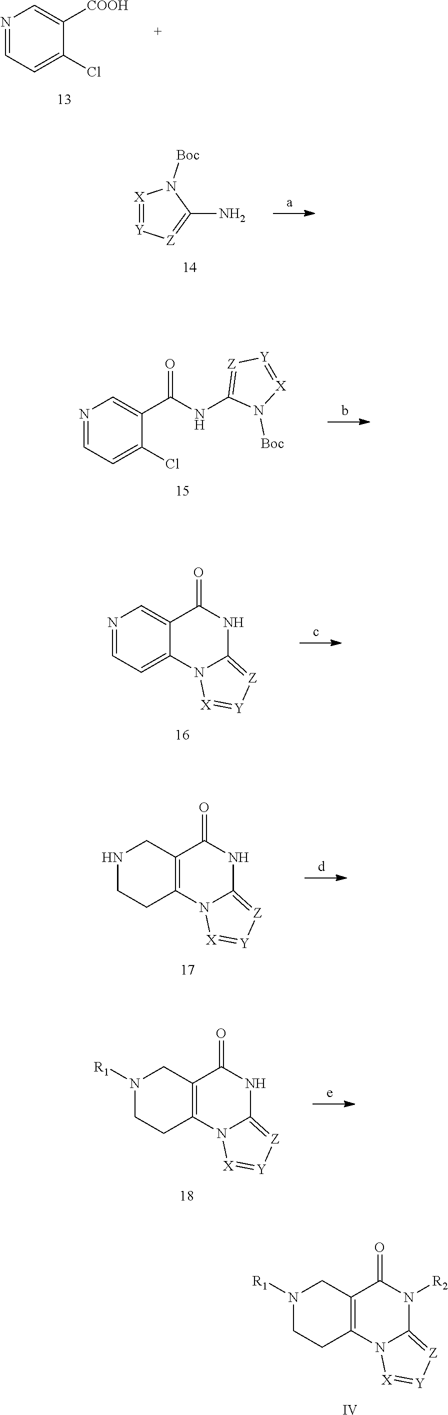

the following steps:
(1) step a: condensing compound 13 with compound 14 to synthesize compound 15; wherein when a five-membered ring of compound 14 contains multiple nitrogen atoms, a Boc protecting group can be on different nitrogen atoms;
(2) step b: removing the Boc protecting group of compound 15 and then performing cyclization through an intramolecular nucleophilic substitution reaction of aryl groups under basic conditions to prepare compound 16;
(3) step c: reducing a pyridine ring in compound 16 by hydrogenation to prepare compound 17;
(4) step d: reductive amination of the amino group in compound 17 with different aromatic aldehydes to synthesize compound 18; and
(5) step e: nucleophilic substitution of compound 18 with different halogenated hydrocarbons to synthesize compounds I-106 to I-162 represented by general formula IV.

When ring A in general formula I is an imidazole ring, a triazole ring or a pyrazole ring in general formula IV, and X, Y and Z are each a nitrogen atom or a methine group, a synthetic route may also comprise

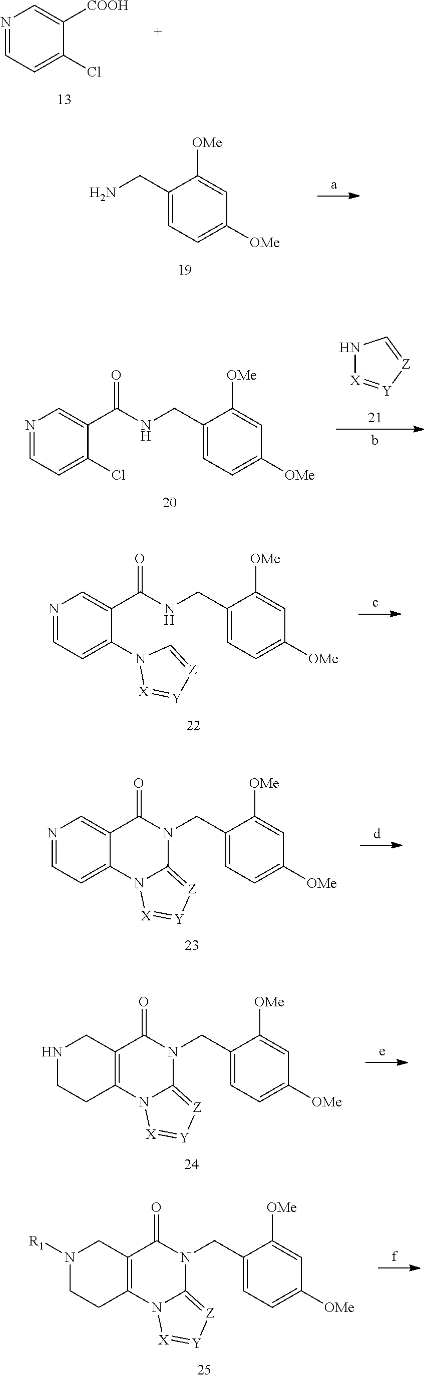

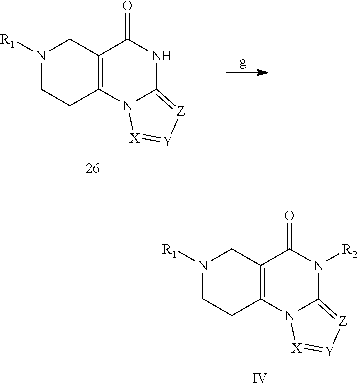

the following steps:
(1) step a: condensing compound 13 with compound 19 to prepare compound 20; (2) step b: nucleophilic substitution of compound 21 with compound 20 under basic conditions to synthesize compound 22;
(3) step c: subjecting compound 22 to intramolecular coupling and cyclization under the catalysis of a cuprous salt to synthesize compound 23;
(4) step d: reducing pyridine in compound 23 under catalytic hydrogenation to synthesize compound 24;
(5) step e: subjecting compound 24 to a nucleophilic substitution reaction with halogenated hydrocarbons to synthesize compound 25;
(6) step f: removing a 2,4-dimethoxybenzyl group of compound 25 under acidic conditions to prepare compound 26;
(7) step g: subjecting compound 26 to a nucleophilic substitution reaction with halogenated hydrocarbons to synthesize compounds represented by general formula IV.

When R₂ in general formula IV does not contain a group that can be reduced by hydrogenation, a synthetic route may also comprise

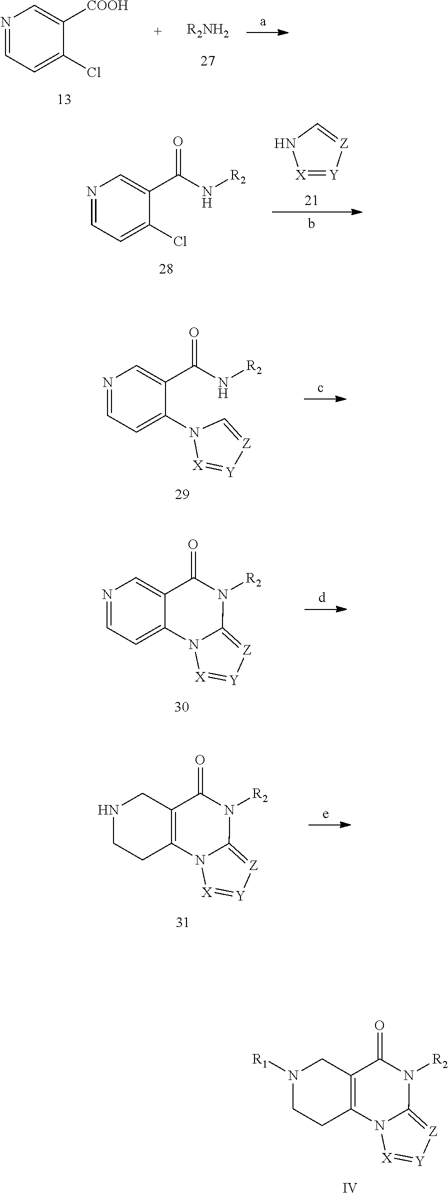

the following steps:
(1) step a: condensing compound 13 with compound 29 to prepare compound 30;
(2) step b: subjecting compound 30 to a nucleophilic substitution reaction of aromatic rings with compound 21 under basic conditions to synthesize compound 31;
(3) step c: subjecting compound 22 to intramolecular coupling and cyclization under the catalysis of a cuprous salt to synthesize compound 32;
(4) step d: reducing pyridine in compound 32 under catalytic hydrogenation to synthesize compound 33; and
(5) step e: subjecting compound 33 to a nucleophilic substitution reaction with halogenated hydrocarbons to synthesize compounds represented by general formula IV.

Provided are pharmaceutical compositions according to the present invention, which comprise the compounds or the pharmaceutically acceptable salts thereof described above.

Use of the compounds of formula I or the pharmaceutically acceptable salts thereof described above and the pharmaceutical compositions described above in the preparation of ClpP agonists are also within the protection scope of the present invention.

Use of the compounds of formula I or the pharmaceutically acceptable salts thereof described above and the pharmaceutical compositions described above in the preparation of medicaments for treating cancer are also within the protection scope of the present invention. The drugs achieve the purpose of treating cancer by activating caseinolytic protease P (ClpP).

Beneficial effects: Through experimental verification, the compounds containing a tetrahydronaphthyridinone or tetrahydropyridopyrimidinone skeleton of the present invention have significant agonistic effects on caseinolytic protease P (ClpP) and can be applied to the treatment of various cancers.

### DETAILED DESCRIPTION OF THE EMBODIMENTS

The content of the present invention will be specifically described below through examples.

### Example 1

### Preparation of ethyl 1-N-tert-butoxycarbonyl-4-piperidone-3-carboxylate (2)

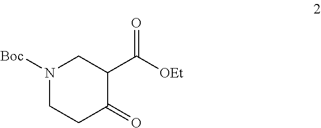 [SMILES: CCOC(=O)C1CN(C(=O)OC(C)(C)C)CCC1=O]

Ethyl 1-benzyl-4-piperidone-3-carboxylate hydrochloride (1) (10 g, 33.6 mmol) was dissolved in 200 mL of ethanol at room temperature, and palladium on carbon (1.0 g, 10% w/w) was added. The reaction system was purged with nitrogen, followed by hydrogen, and then subjected to a hydrogenation reaction at 1 atm for 12 h. After the reaction was completed as monitored by TLC, di-tert-butyl dicarbonate (8.1 g, 37.0 mmol) and triethylamine (10.3 mL, 73.9 mmol) were added, and the mixture was stirred for reaction at room temperature for 8 h. After the reaction was completed as monitored by TLC, the palladium on carbon was filtered out through celite, and the filtrate was concentrated by rotary evaporation under reduced pressure to remove the solvent. 200 mL of water was added, and the resulting mixture was extracted with dichloromethane (150 mL×3). The organic phases were combined, washed with saturated brine, dried over anhydrous sodium sulfate, concentrated by rotary evaporation under reduced pressure to remove the solvent, and purified by silica gel column chromatography to give a colorless oily liquid (8.6 g, yield: 94%). 1H NMR (300 MHz, Chloroform-d) δ 4.23 (q, J=6.8 Hz, 2H), 4.08 (s, 2H), 3.28 (t, J=5.8 Hz, 2H), 2.37 (t, J=5.8 Hz, 2H), 1.58 (s, 9H), 1.32 (t, J=6.8 Hz, 3H).

### Example 2

### Preparation of 1-(tert-butyl)-3-ethyl-4-(((trifluoromethyl)sulfonyl)oxy)-5,6-dihydro-1,3(2H)-pyridinedicarboxylate (3)

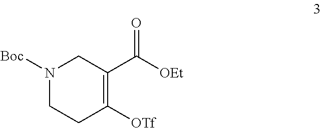 [SMILES: CCOC(=O)C1=C(OS(=O)(=O)C(F)(F)F)CCN(C(=O)OC(C)(C)C)C1]

Ethyl 1-N-tert-butoxycarbonyl-4-piperidone-3-carboxylate 2 (7.5 g, 27.7 mmol) was dissolved in 150 mL of dichloromethane at room temperature, and N,N-diisopropylethylamine (11.7 mL, 82.8 mmol) was added under argon atmosphere. The reaction system was cooled to −78° C., and trifluoromethanesulfonic anhydride (6.0 mL, 35.9 mmol) was slowly added dropwise. After stirring for 1 h, the reaction system was warmed to 0° C., and stirred at this temperature for 23 h. After the reaction was completed as monitored by TLC, the reaction was quenched by adding 100 mL of saturated sodium bicarbonate solution. The aqueous phase was extracted with dichloromethane (50 mL×3). The organic phases were combined, washed with saturated brine, dried over anhydrous sodium sulfate, concentrated by rotary evaporation under reduced pressure to remove the solvent, and purified by silica gel column chromatography to give a colorless oily liquid (6.5 g, yield: 58%). ¹H NMR (300 MHz, Chloroform-d) δ 4.38-4.24 (m, 4H), 3.63 (t, J=5.7 Hz, 2H), 2.57-2.46 (m, 2H), 1.49 (s, 9H), 1.34 (t, J=7.1 Hz, 3H).

### Example 3

### Preparation of 1-(tert-butyl)-3-ethyl-4-(4,4,5,5-tetramethyl-1,3,2-dioxaboran-2-yl)-5,6-dihydro-1,3(2H)-pyridine dicarboxylate (4)

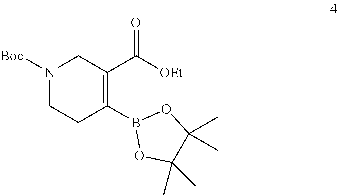 [SMILES: CCOC(=O)C1=C(B2OC(C)(C)C(C)(C)O2)CCN(C(=O)OC(C)(C)C)C1]

1-(Tert-butyl)-3-ethyl-4-(((trifluoromethyl)sulfonyl)oxy)-5,6-dihydro-1,3(2H)-pyridine dicarboxylate 3 (6.5 g, 16.1 mmol) was dissolved in 130 mL of 1,4-dioxane at room temperature, and bis(pinacolato)diboron (4.9 g, 19.4 mmol), [1,1′-bis(diphenylphosphino)ferrocene]dichloropalladium(II) (1.18 g, 1.61 mmol), 2-dicyclohexylphosphine-2′,6′-dimethoxybiphenyl (661 mg, 1.61 mmol), and potassium acetate (3.16 g, 32.2 mmol) were added under argon atmosphere. The mixture was stirred and reacted at reflux overnight. After the reaction was completed as monitored by TLC, the reaction mixture was cooled to room temperature, filtered to remove the solid, concentrated by rotary evaporation under reduced pressure to remove the solvent, and purified by silica gel column chromatography to give a colorless oily liquid (3.6 g, yield: 54%). ¹H NMR (300 MHz, Chloroform-d) δ 4.20-3.98 (m, 4H), 3.36 (t, J=5.4 Hz, 2H), 2.28-2.20 (m, 2H), 1.38 (s, 9H), 1.26 (s, 12H), 1.24 (t, J=6.5 Hz, 3H).

### Example 4

### Preparation of tert-butyl 5-oxo-1,4,5,6-tetrahydropyrido[3,4-c][1,8]naphthyridine-3-(2H)-carboxylate (5-A)

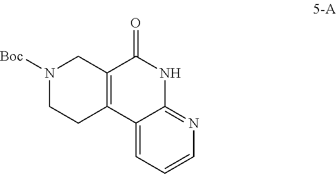 [SMILES: CC(C)(C)OC(=O)N1CCc2c(c(=O)[nH]c3ncccc23)C1]

1-(Tert-butyl)-3-ethyl-4-(4,4,5,5-tetramethyl-1,3,2-dioxaboran-2-yl)-5,6-dihydro-1,3(2H)-pyridine dicarboxylate (4) (3 g, 7.87 mmol) was dissolved in a mixed solvent of 48 mL of 1,4-dioxane and 8 mL of water at room temperature, and 2-amino-3-iodopyridine (2.60 g, 11.8 mmol), [1,1′-bis(diphenylphosphino)ferrocene]dichloropalladium(II) (578 mg, 0.79 mmol), 2-dicyclohexylphosphine-2′,6′-dimethoxybiphenyl (324 mg, 0.79 mmol), and cesium carbonate (3.84 g, 11.8 mmol) were added. The mixture was stirred and reacted at reflux overnight under argon atmosphere. After the reaction was completed as monitored by TLC, the reaction mixture was cooled to room temperature, filtered to remove the solid, concentrated by rotary evaporation under reduced pressure to remove the solvent, and purified by silica gel column chromatography to give a white solid (1.9 g, yield: 80%). ¹H NMR (300 MHz, Chloroform-d) δ 13.16 (s, 1H), 8.75 (d, J=3.6 Hz, 1H), 8.00 (d, J=7.7 Hz, 1H), 7.26 (dd, J=7.9, 4.8 Hz, 1H), 4.51 (s, 2H), 3.78 (t, J=5.6 Hz, 2H), 2.93 (t, J=5.7 Hz, 2H), 1.52 (s, 9H).

### Example 5

### Preparation of tert-butyl 5-oxo-1,4,5,6-benzo[c][2,7]naphthyridine-3-(2H)-carboxylate (5-B)

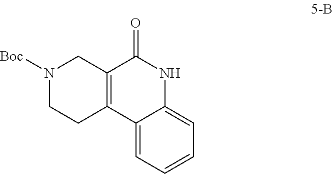 [SMILES: CC(C)(C)OC(=O)N1CCc2c(c(=O)[nH]c3ccccc23)C1]

Compound 5-B was synthesized by referring to the preparation method for the compound tert-butyl 5-oxo-1,4,5,6-tetrahydropyrido[3,4-c][1,8]naphthyridine-3-(2H)-carboxylic acid (5-A) in Example 4, with 1-(tert-butyl)-3-ethyl-4-(4,4,5,5-tetramethyl-1,3,2-dioxaboran-2-yl)-5,6-dihydro-1,3(2H)-pyridine dicarboxylate (4) and 2-iodoaniline as starting materials. White solid, ¹H NMR (300 MHz, Chloroform-d) δ 12.54 (s, 1H), 8.42 (d, J=3.6 Hz, 1H), 7.62 (d, J=7.7 Hz, 1H), 7.13 (dd, J=7.9, 4.8 Hz, 1H), 4.46 (s, 2H), 3.82 (t, J=5.5 Hz, 2H), 2.91 (t, J=5.6 Hz, 2H), 1.52 (s, 9H).

### Example 6

### Preparation of tert-butyl 6-oxo-5,7,9,10-tetrahydropyrimidinyl[4,5-c][2,7]naphthyridine-8-(6H)-carboxylate (5-C)

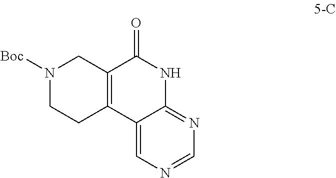 [SMILES: CC(C)(C)OC(=O)N1CCc2c(c(=O)[nH]c3ncncc23)C1]

Compound 5-C was synthesized by referring to the synthetic method for the compound tert-butyl 5-oxo-1,4,5,6-tetrahydropyrido[3,4-c][1,8]naphthyridine-3-(2H)-carboxylate (5-A) in Example 4, with 1-(tert-butyl)-3-ethyl-4-(4,4,5,5-tetramethyl-1,3,2-dioxaboran-2-yl)-5,6-dihydro-1,3(2H)-pyridinedicarboxylate (4) and 4-amino-5-iodopyrimidine as starting materials. White solid, ¹H NMR (300 MHz, Chloroform-d) δ 11.27 (s, 1H), 9.13 (s, 1H), 9.00 (s, 1H), 4.52 (s, 2H), 3.79 (t, J=5.7 Hz, 2H), 3.00 (t, J=5.5 Hz, 2H), 1.51 (s, 9H).

### Example 7

### Preparation of tert-butyl 5-oxo-4,5,8,9-tetrahydrofuro[3,4-c][2,7]naphthyridine-7-(6H)-carboxylate (5-D)

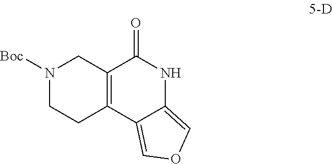 [SMILES: CC(C)(C)OC(=O)N1CCc2c(c(=O)[nH]c3cocc23)C1]

Compound 5-D was synthesized by referring to the synthetic method for the compound tert-butyl 5-oxo-1,4,5,6-tetrahydropyrido[3,4-c][1,8]naphthyridine-3-(2H)-carboxylate (5-A) in Example 4, with 1-(tert-butyl)-3-ethyl-4-(4,4,5,5-tetramethyl-1,3,2-dioxaboran-2-yl)-5,6-dihydro-1,3(2H)-pyridine dicarboxylate (4) and 3-amino-4-iodofuran as starting materials. White solid, MS (ESI) m/z: 291.2 [M+H]⁺.

### Example 8

### Preparation of tert-butyl 5-oxo-4,5,8,9-tetrahydrothieno[3,4-c][2,7]naphthyridine-7-(6H)-carboxylate (5-E)

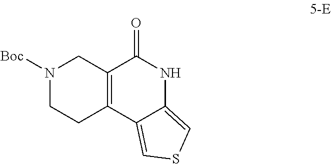 [SMILES: CC(C)(C)OC(=O)N1CCc2c(c(=O)[nH]c3cscc23)C1]

Compound 5-E was synthesized by referring to the synthetic method for the compound tert-butyl 5-oxo-1,4,5,6-tetrahydropyrido[3,4-c][1,8]naphthyridine-3-(2H)-carboxylate (5-A) in Example 4, with 1-(tert-butyl)-3-ethyl-4-(4,4,5,5-tetramethyl-1,3,2-dioxaboran-2-yl)-5,6-dihydro-1,3(2H)-pyridine dicarboxylate (4) and 3-amino-4-iodothiophene as starting materials. White solid, MS (ESI) m/z: 307.2 [M+H]⁺.

### Example 9

### Preparation of tert-butyl 5-oxo-4,5,8,9-tetrahydrothieno[3,2-c][2,7]naphthyridine-7-(6H)-carboxylate (5-F)

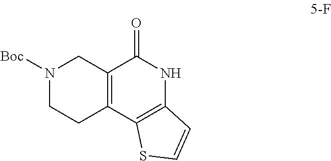 [SMILES: CC(C)(C)OC(=O)N1CCc2c(c(=O)[nH]c3ccsc23)C1]

Compound 5-F was synthesized by referring to the synthetic method for the compound tert-butyl 5-oxo-1,4,5,6-tetrahydropyrido[3,4-c][1,8]naphthyridine-3-(2H)-carboxylate (5-A) in Example 4, with 1-(tert-butyl)-3-ethyl-4-(4,4,5,5-tetramethyl-1,3,2-dioxaboran-2-yl)-5,6-dihydro-1,3(2H)-pyridine dicarboxylate (4) and 3-amino-2-iodothiophene as starting materials. White solid, MS (ESI) m/z: 307.2 [M+H]⁺.

### Example 10

### Preparation of tert-butyl 5-oxo-4,5,8,9-tetrahydrothieno[2,3-c][2,7]naphthyridine-7-(6H)-carboxylate (5-G)

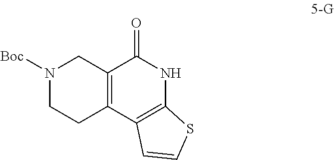 [SMILES: CC(C)(C)OC(=O)N1CCc2c(c(=O)[nH]c3sccc23)C1]

Compound 5-G was synthesized by referring to the synthetic method for the compound tert-butyl 5-oxo-1,4,5,6-tetrahydropyrido[3,4-c][1,8]naphthyridine-3-(2H)-carboxylate (5-A) in Example 4, with 1-(tert-butyl)-3-ethyl-4-(4,4,5,5-tetramethyl-1,3,2-dioxaboran-2-yl)-5,6-dihydro-1,3(2H)-pyridine dicarboxylate (4) and 2-amino-3-iodothiophene as starting materials. White solid, MS (ESI) m/z: 307.2 [M+H]⁺.

### Example 11

### Preparation of tert-butyl 5-oxo-4,6,8,9-tetrahydrothiazolo[4,5-c][2,7]naphthyridine-7-(5H)-carboxylate (5-H)

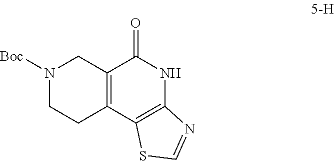 [SMILES: CC(C)(C)OC(=O)N1CCc2c(c(=O)[nH]c3ncsc23)C1]

Compound 5-H was synthesized by referring to the synthetic method for the compound tert-butyl 5-oxo-1,4,5,6-tetrahydropyrido[3,4-c][1,8]naphthyridine-3-(2H)-carboxylate (5-A) in Example 4, with 1-(tert-butyl)-3-ethyl-4-(4,4,5,5-tetramethyl-1,3,2-dioxaboran-2-yl)-5,6-dihydro-1,3(2H)-pyridinedicarboxylate (4) and 4-amino-5-iodothiazole as starting materials. White solid, MS (ESI) m/z: 308.2 [M+H]⁺.

### Example 12

### Preparation of tert-butyl 6-benzyl-5-oxo-1,4,5,6-tetrahydropyrido[3,4-c][1,8]naphthyridine-3(2H)-carboxylate (6-A-1)

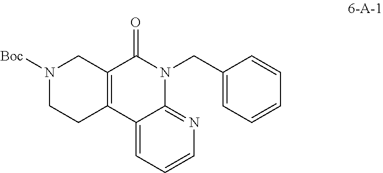 [SMILES: CC(C)(C)OC(=O)N1CCc2c(c(=O)n(Cc3ccccc3)c3ncccc23)C1]

Compound 5-A-1 (600 mg, 1.99 mmol) was dissolved in 10 mL of N,N-dimethylformamide at room temperature, and potassium carbonate (275 mg, 3.98 mmol) was added, followed by dropwise addition of benzyl bromide (681 mg, 3.98 mmol). The mixture was stirred overnight. After the reaction was completed as monitored by TLC, 50 mL of water was added, and the mixture was extracted with ethyl acetate (30 mL×3). The organic phases were combined, washed with saturated brine, dried over anhydrous sodium sulfate, concentrated by rotary evaporation under reduced pressure to remove the solvent, and purified by silica gel column chromatography to give a white solid (545 mg, yield: 70%). ¹H NMR (300 MHz, Chloroform-d) δ 8.55 (dd, J=4.6, 1.7 Hz, 1H), 7.91 (dd, J=7.9, 1.7 Hz, 1H), 7.48-7.43 (m, 2H), 7.27 (d, J=4.3 Hz, 1H), 7.24-7.13 (m, 3H), 5.77 (s, 2H), 3.58 (s, 2H), 2.93 (t, J=5.6 Hz, 2H), 2.78 (t, J=5.7 Hz, 2H), 1.43 (s, 9H); MS (ESI) m/z: 392.2 [M+H]⁺.

### Example 13

### Preparation of tert-butyl 6-cyclopropylmethyl-5-oxo-1,4,5,6-tetrahydropyrido[3,4-c][1,8]naphthyridine-3(2H)-carboxylate (6-A-2)

The compound was prepared by referring to the preparation method for compound 6-A-1 in Example 12, with compound 5-A and (bromomethyl)cyclopropane as starting materials. MS (ESI) m/z: 356.2 [M+H]⁺.

### Example 14

### Preparation of tert-butyl 6-cyclobutylmethyl-5-oxo-1,4,5,6-tetrahydropyrido[3,4-c][1,8]naphthyridine-3(2H)-carboxylate (6-A-3)

The compound was prepared by referring to the preparation method for compound 6-A-1 in Example 12, with compound 5-A and (bromomethyl)cyclobutane as starting materials. MS (ESI) m/z: 370.2 [M+H]⁺.

### Example 15

### Preparation of tert-butyl 6-cyclopentylmethyl-5-oxo-1,4,5,6-tetrahydropyrido[3,4-c][1,8]naphthyridine-3(2H)-carboxylate (6-A-4)

The compound was prepared by referring to the preparation method for compound 6-A-1 in Example 12, with compound 5-A and (bromomethyl)cyclopentane as starting materials. MS (ESI) m/z: 384.2 [M+H]⁺.

### Example 16

### Preparation of tert-butyl 6-cyclohexylmethyl-5-oxo-1,4,5,6-tetrahydropyrido[3,4-c][1,8]naphthyridine-3(2H)-carboxylate (6-A-5)

The compound was prepared by referring to the preparation method for compound 6-A-1 in Example 12, with compound 5-A and (bromomethyl)cyclohexane as starting materials. MS (ESI) m/z: 398.2 [M+H]⁺.

### Example 17

### Preparation of tert-butyl 6-(2-fluorobenzyl)-5-oxo-1,4,5,6-tetrahydropyrido[3,4-c][1,8]naphthyridine-3(2H)-carboxylate (6-A-6)

The compound was prepared by referring to the preparation method for compound 6-A-1 in Example 12, with compound 5-A and 2-fluorobenzyl bromide as starting materials. MS (ESI) m/z: 410.2 [M+H]⁺.

### Example 18

### Preparation of tert-butyl 6-(2-chlorobenzyl)-5-oxo-1,4,5,6-tetrahydropyrido[3,4-c][1,8]naphthyridine-3(2H)-carboxylate (6-A-7)

The compound was prepared by referring to the preparation method for compound 6-A-1 in Example 12, with compound 5-A and 2-chlorobenzyl bromide as starting materials. MS (ESI) m/z: 426.2 [M+H]⁺.

### Example 19

### Preparation of tert-butyl 6-(2-bromobenzyl)-5-oxo-1,4,5,6-tetrahydropyrido[3,4-c][1,8]naphthyridine-3(2H)-carboxylate (6-A-8)

The compound was prepared by referring to the preparation method for compound 6-A-1 in Example 12, with compound 5-A and 2-bromobenzyl bromide as starting materials. MS (ESI) m/z: 471.1 [M+H]⁺.

### Example 20

### Preparation of tert-butyl 6-(2-methylbenzyl)-5-oxo-1,4,5,6-tetrahydropyrido[3,4-c][1,8]naphthyridine-3(2H)-carboxylate (6-A-9)

The compound was prepared by referring to the preparation method for compound 6-A-1 in Example 12, with compound 5-A and 2-methylbenzyl bromide as starting materials. MS (ESI) m/z: 406.2 [M+H]⁺.

### Example 21

### Preparation of tert-butyl 6-(2-ethylbenzyl)-5-oxo-1,4,5,6-tetrahydropyrido[3,4-c][1,8]naphthyridine-3(2H)-carboxylate (6-A-10)

The compound was prepared by referring to the preparation method for compound 6-A-1 in Example 12, with compound 5-A and 2-ethylbenzyl bromide as starting materials. MS (ESI) m/z: 420.2 [M+H]⁺.

### Example 22

### Preparation of tert-butyl 6-(2-methoxybenzyl)-5-oxo-1,4,5,6-tetrahydropyrido[3,4-c][1,8]naphthyridine-3(2H)-carboxylate (6-A-11)

The compound was prepared by referring to the preparation method for compound 6-A-1 in Example 12, with compound 5-A and 2-ethylbenzyl bromide as starting materials. MS (ESI) m/z: 422.2 [M+H]⁺.

### Example 23

### Preparation of tert-butyl 6-(3-fluorobenzyl)-5-oxo-1,4,5,6-tetrahydropyrido[3,4-c][1,8]naphthyridine-3(2H)-carboxylate (6-A-12)

The compound was prepared by referring to the preparation method for compound 6-A-1 in Example 12, with compound 5-A and 2-fluorobenzyl bromide as starting materials. MS (ESI) m/z: 410.2 [M+H]⁺.

### Example 24

### Preparation of tert-butyl 6-(3-chlorobenzyl)-5-oxo-1,4,5,6-tetrahydropyrido[3,4-c][1,8]naphthyridine-3(2H)-carboxylate (6-A-13)

The compound was prepared by referring to the preparation method for compound 6-A-1 in Example 12, with compound 5-A and 2-chlorobenzyl bromide as starting materials. MS (ESI) m/z: 426.2 [M+H]⁺.

### Example 25

### Preparation of tert-butyl 6-(3-bromobenzyl)-5-oxo-1,4,5,6-tetrahydropyrido[3,4-c][1,8]naphthyridine-3(2H)-carboxylate (6-A-14)

The compound was prepared by referring to the preparation method for compound 6-A-1 in Example 12, with compound 5-A and 2-bromobenzyl bromide as starting materials. MS (ESI) m/z: 471.1 [M+H]⁺.

### Example 26

### Preparation of tert-butyl 6-(3-methylbenzyl)-5-oxo-1,4,5,6-tetrahydropyrido[3,4-c][1,8]naphthyridine-3(2H)-carboxylate (6-A-15)

The compound was prepared by referring to the preparation method for compound 6-A-1 in Example 12, with compound 5-A and 2-methylbenzyl bromide as starting materials. MS (ESI) m/z: 406.2 [M+H]⁺.

### Example 27

### Preparation of tert-butyl 6-(3-ethylbenzyl)-5-oxo-1,4,5,6-tetrahydropyrido[3,4-c][1,8]naphthyridine-3(2H)-carboxylate (6-A-16)

The compound was prepared by referring to the preparation method for compound 6-A-1 in Example 12, with compound 5-A and 2-ethylbenzyl bromide as starting materials. MS (ESI) m/z: 420.2 [M+H]⁺.

### Preparation of tert-butyl 6-(3-methoxybenzyl)-5-oxo-1,4,5,6-tetrahydropyrido[3,4-c][1,8]naphthyridine-3(2H)-carboxylate (6-A-17)

The compound was prepared by referring to the preparation method for compound 6-A-1 in Example 12, with compound 5-A and 2-ethylbenzyl bromide as starting materials. MS (ESI) m/z: 422.2 [M+H]⁺.

### Example 29

### Preparation of tert-butyl 6-(4-fluorobenzyl)-5-oxo-1,4,5,6-tetrahydropyrido[3,4-c][1,8]naphthyridine-3(2H)-carboxylate (6-A-18)

The compound was prepared by referring to the preparation method for compound 6-A-1 in Example 12, with compound 5-A and 2-fluorobenzyl bromide as starting materials. MS (ESI) m/z: 410.2 [M+H]⁺.

### Example 30

### Preparation of tert-butyl 6-(4-chlorobenzyl)-5-oxo-1,4,5,6-tetrahydropyrido[3,4-c][1,8]naphthyridine-3(2H)-carboxylate (6-A-19)

The compound was prepared by referring to the preparation method for compound 6-A-1 in Example 12, with compound 5-A and 2-chlorobenzyl bromide as starting materials. MS (ESI) m/z: 426.2 [M+H]⁺.

### Example 31

### Preparation of tert-butyl 6-(4-bromobenzyl)-5-oxo-1,4,5,6-tetrahydropyrido[3,4-c][1,8]naphthyridine-3(2H)-carboxylate (6-A-20)

The compound was prepared by referring to the preparation method for compound 6-A-1 in Example 12, with compound 5-A and 2-bromobenzyl bromide as starting materials. MS (ESI) m/z: 471.1 [M+H]⁺.

### Example 32

### Preparation of tert-butyl 6-(4-methylbenzyl)-5-oxo-1,4,5,6-tetrahydropyrido[3,4-c][1,8]naphthyridine-3(2H)-carboxylate (6-A-21)

The compound was prepared by referring to the preparation method for compound 6-A-1 in Example 12, with compound 5-A and 2-methylbenzyl bromide as starting materials. MS (ESI) m/z: 406.2 [M+H]⁺.

### Example 33

### Preparation of tert-butyl 6-(4-ethylbenzyl)-5-oxo-1,4,5,6-tetrahydropyrido[3,4-c][1,8]naphthyridine-3(2H)-carboxylate (6-A-22)

The compound was prepared by referring to the preparation method for compound 6-A-1 in Example 12, with compound 5-A and 2-ethylbenzyl bromide as starting materials. MS (ESI) m/z: 420.2 [M+H]⁺.

### Example 34

### Preparation of tert-butyl 6-(4-methoxybenzyl)-5-oxo-1,4,5,6-tetrahydropyrido[3,4-c][1,8]naphthyridine-3(2H)-carboxylate (6-A-23)

The compound was prepared by referring to the preparation method for compound 6-A-1 in Example 12, with compound 5-A and 2-ethylbenzyl bromide as starting materials. MS (ESI) m/z: 422.2 [M+H]⁺.

### Example 35

### Preparation of tert-butyl 6-(4-trifluoromethylbenzyl)-5-oxo-1,4,5,6-tetrahydropyrido[3,4-c][1,8]naphthyridine-3(2H)-carboxylate (6-A-24)

The compound was prepared by referring to the preparation method for compound 6-A-1 in Example 12, with compound 5-A and 4-trifluoromethylbenzyl bromide as starting materials. MS (ESI) m/z: 460.2 [M+H]⁺.

### Example 36

### Preparation of tert-butyl 6-((1-methyl-1H-pyrazol-3-yl)methyl)-5-oxo-1,4,5,6-tetrahydropyrido[3,4-c][1,8]naphthyridine-3(2H)-carboxylate (6-A-25)

The compound was prepared by referring to the preparation method for compound 6-A-1 in Example 12, with compound 5-A and 3-bromomethyl-1-methyl-1H-pyrazole as starting materials. MS (ESI) m/z: 396.2 [M+H]⁺.

### Example 37

### Preparation of tert-butyl 6-((1-methyl-1H-pyrazol-5-yl)methyl)-5-oxo-1,4,5,6-tetrahydropyrido[3,4-c][1,8]naphthyridine-3(2H)-carboxylate (6-A-26)

The compound was prepared by referring to the preparation method for compound 6-A-1 in Example 12, with compound 5-A and 5-bromomethyl-1-methyl-1H-pyrazole as starting materials. MS (ESI) m/z: 396.2 [M+H]⁺.

### Example 38

### Preparation of tert-butyl 6-((1,3-dimethyl-1H-pyrazol-5-yl)methyl)-5-oxo-1,4,5,6-tetrahydropyrido[3,4-c][1,8]naphthyridine-3(2H)-carboxylate (6-A-27)

The compound was prepared by referring to the preparation method for compound 6-A-1 in Example 12, with compound 5-A and 5-bromomethyl-1,3-dimethyl-1H-pyrazole as starting materials. MS (ESI) m/z: 410.2 [M+H]⁺.

### Preparation of tert-butyl 6-(pyridin-2-ylmethyl)-5-oxo-1,4,5,6-tetrahydropyrido[3,4-c][1,8]naphthyridine-3(2H)-carboxylate (6-A-28)

The compound was prepared by referring to the preparation method for compound 6-A-1 in Example 12, with compound 5-A and 2-chloromethylpyridine as starting materials. MS (ESI) m/z: 393.2 [M+H]⁺.

### Example 40

### Preparation of tert-butyl 6-(pyridin-3-ylmethyl)-5-oxo-1,4,5,6-tetrahydropyrido[3,4-c][1,8]naphthyridine-3(2H)-carboxylate (6-A-29)

The compound was prepared by referring to the preparation method for compound 6-A-1 in Example 12, with compound 5-A and 3-chloromethylpyridine as starting materials. MS (ESI) m/z: 393.2 [M+H]⁺.

### Example 41

### Preparation of tert-butyl 6-(pyridin-4-ylmethyl)-5-oxo-1,4,5,6-tetrahydropyrido[3,4-c][1,8]naphthyridine-3(2H)-carboxylate (6-A-30)

The compound was prepared by referring to the preparation method for compound 6-A-1 in Example 12, with compound 5-A and 4-chloromethylpyridine as starting materials. MS (ESI) m/z: 393.2 [M+H]⁺.

### Example 42

### Preparation of tert-butyl 6-((4-chloropyridin-2-yl)methyl)-5-oxo-1,4,5,6-tetrahydropyrido[3,4-c][1,8]naphthyridine-3(2H)-carboxylate (6-A-31)

The compound was prepared by referring to the preparation method for compound 6-A-1 in Example 12, with compound 5-A and 2-chloromethyl-4-chloropyridine as starting materials. MS (ESI) m/z: 427.2 [M+H]⁺.

### Example 43

### Preparation of tert-butyl 6-((1-methyl-1H-imidazol-2-yl)methyl)-5-oxo-1,4,5,6-tetrahydropyrido[3,4-c][1,8]naphthyridine-3(2H)-carboxylate (6-A-32)

The compound was prepared by referring to the preparation method for compound 6-A-1 in Example 12, with compound 5-A and 2-chloromethyl-1-methyl-1H-imidazole as starting materials. MS (ESI) m/z: 396.2 [M+H]⁺.

### Example 44

### Preparation of tert-butyl 6-((1-methyl-1H-imidazol-4-yl)methyl)-5-oxo-1,4,5,6-tetrahydropyrido[3,4-c][1,8]naphthyridine-3(2H)-carboxylate (6-A-33)

The compound was prepared by referring to the preparation method for compound 6-A-1 in Example 12, with compound 5-A and 4-chloromethyl-1-methyl-1H-imidazole as starting materials. MS (ESI) m/z: 396.2 [M+H]⁺.

### Example 45

### Preparation of tert-butyl 6-((1-methyl-1H-imidazol-5-yl)methyl)-5-oxo-1,4,5,6-tetrahydropyrido[3,4-c][1,8]naphthyridine-3(2H)-carboxylate (6-A-34)

The compound was prepared by referring to the preparation method for compound 6-A-1 in Example 12, with compound 5-A and 5-chloromethyl-1-methyl-1H-imidazole as starting materials. MS (ESI) m/z: 396.2 [M+H]⁺.

### Example 46

### Preparation of tert-butyl 6-((3,5-dimethylisoxazol-4-yl)methyl)-5-oxo-1,4,5,6-tetrahydropyrido[3,4-c][1,8]naphthyridine-3(2H)-carboxylate (6-A-35)

The compound was prepared by referring to the preparation method for compound 6-A-1 in Example 12, with compound 5-A and 4-chloromethyl-3,5-dimethylisoxazole as starting materials. MS (ESI) m/z: 411.2 [M+H]⁺.

### Example 47

### Preparation of tert-butyl 6-((2-methylthiazol-5-yl)methyl)-5-oxo-1,4,5,6-tetrahydropyrido[3,4-c][1,8]naphthyridine-3(2H)-carboxylate (6-A-36)

The compound was prepared by referring to the preparation method for compound 6-A-1 in Example 12, with compound 5-A and 5-chloromethyl-2-methylthiazole as starting materials. MS (ESI) m/z: 413.2 [M+H]⁺.

### Example 48

### Preparation of tert-butyl 6-(4-nitrobenzyl)-5-oxo-1,4,5,6-tetrahydropyrido[3,4-c][1,8]naphthyridine-3(2H)-carboxylate (6-A-37)

The compound was prepared by referring to the preparation method for compound 6-A-1 in Example 12, with compound 5-A and 4-nitrobenzyl bromide as starting materials. MS (ESI) m/z: 437.4 [M+H]⁺.

### Example 49

### Preparation of tert-butyl 6-((2,3-dihydro[1,4]benzodioxin-6-yl)methyl)-5-oxo-1,4,5,6-tetrahydropyrido[3,4-c][1,8]naphthyridine-3(2H)-carboxylate (6-A-38)

The compound was prepared by referring to the preparation method for compound 6-A-1 in Example 12, with compound 5-A and 6-bromomethyl-2,3-dihydro[1,4]benzodioxine as starting materials. MS (ESI) m/z: 450.4 [M+H]⁺.

### Example 50

### Preparation of tert-butyl 6-(benzo[1,3]dioxopentacyclo-5-yl)methyl)-5-oxo-1,4,5,6-tetrahydropyrido[3,4-c][1,8]naphthyridine-3(2H)-carboxylate (6-A-39)

The compound was prepared by referring to the preparation method for compound 6-A-1 in Example 12, with compound 5-A and 5-bromomethyl-benzo[1,3]dioxolane as starting materials. MS (ESI) m/z: 436.3 [M+H]⁺.

### Example 51

### Preparation of tert-butyl 6-(4-(trifluoromethyl)benzyl)-5-oxo-1,4,5,6-tetrahydrobenzo[c][2,7]naphthyridine-3-(2H)-carboxylate (6-B-1)

The compound was prepared by referring to the preparation method for compound 6-A-1 in Example 12, with compound 5-B and 4-(trifluoromethyl)benzyl bromide as starting materials. MS (ESI) m/z: 459.2 [M+H]⁺.

### Example 52

### Preparation of tert-butyl 5-(4-(trifluoromethyl)benzyl)-6-oxo-5,7,9,10-tetrahydropyrimidino[4,5-c][2,7]naphthyridine-8-(6H)-carboxylate (6-C-1)

The compound was prepared by referring to the preparation method for compound 6-A-1 in Example 12, with compound 5-C and 4-(trifluoromethyl)benzyl bromide as starting materials. MS (ESI) m/z: 461.2 [M+H]⁺.

### Example 53

### Preparation of tert-butyl 5-oxo-4-(4-trifluoromethylbenzyl)-4,5,8,9-tetrahydrofuro[3,4-c][2,7]naphthyridine-7-(6H)-carboxylate (6-D)

The compound was prepared by referring to the preparation method for compound 6-A-1 in Example 12, with compound 5-D and 4-(trifluoromethyl)benzyl bromide as starting materials. MS (ESI) m/z: 449.3 [M+H]⁺.

### Example 54

### Preparation of tert-butyl 5-oxo-4-(4-trifluoromethylbenzyl)-4,5,8,9-tetrahydrothieno[3,4-c][2,7]naphthyridine-7-(6H)-carboxylate (6-E)

The compound was prepared by referring to the preparation method for compound 6-A-1 in Example 12, with compound 5-E and 4-(trifluoromethyl)benzyl bromide as starting materials. MS (ESI) m/z: 465.3 [M+H]⁺.

### Example 55

### Preparation of tert-butyl 5-oxo-4-(4-trifluoromethylbenzyl)-4,5,8,9-tetrahydrothieno[3,2-c][2,7]naphthyridine-7-(6H)-carboxylate (6-F)

The compound was prepared by referring to the preparation method for compound 6-A-1 in Example 12, with compound 5-F and 4-(trifluoromethyl)benzyl bromide as starting materials. MS (ESI) m/z: 465.3 [M+H]⁺.

### Example 56

### Preparation of tert-butyl 5-oxo-4-(4-trifluoromethylbenzyl)-4,5,8,9-tetrahydrothieno[2,3-c][2,7]naphthyridine-7-(6H)-carboxylate (6-G)

The compound was prepared by referring to the preparation method for compound 6-A-1 in Example 12, with compound 5-G and 4-(trifluoromethyl)benzyl bromide as starting materials. MS (ESI) m/z: 465.3 [M+H]⁺.

### Example 57

### Preparation of tert-butyl 5-oxo-4-(4-trifluoromethylbenzyl)-4,6,8,9-tetrahydrothiazolo[4,5-c][2,7]naphthyridine-7-(5H)-carboxylate (6-H)

The compound was prepared by referring to the preparation method for compound 6-A-1 in Example 12, with compound 5-H and 4-(trifluoromethyl)benzyl bromide as starting materials. MS (ESI) m/z: 466.3 [M+H]⁺.

### Example 58

### Preparation of 6-benzyl-2,3,4,6-tetrahydropyrido[3,4-c][1,8]naphthyridine-5-(H)-one (7-1)

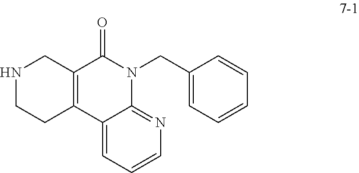 [SMILES: O=c1c2c(c3cccnc3n1Cc1ccccc1)CCNC2]

Tert-butyl 6-benzyl-5-oxo-1,4,5,6-tetrahydropyrido[3,4-c][1,8]naphthyridine-3(2H)-carboxylate (6-A-1) (500 mg, 1.28 mmol) was dissolved in 3 mL of dichloromethane at room temperature, and 6 mL of trifluoroacetic acid was added. The mixture was stirred at room temperature for 1 h. After the reaction was completed as monitored by TLC, the reaction mixture was concentrated by rotary evaporation under reduced pressure to remove the solvent, neutralized with a sodium bicarbonate solution to pH 8, extracted with dichloromethane, dried over anhydrous sodium sulfate, and concentrated to give a yellow viscous liquid (335 mg, yield: 90%); MS (ESI) m/z: 292.2 [M+H]⁺.

### Example 59

### Preparation of 6-benzyl-3-(cyclopropylmethyl)-2,3,4,6-tetrahydropyrido[3,4-c][1,8]naphthyridine-5-(1H)-one (I-1)

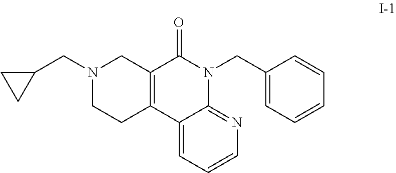 [SMILES: O=c1c2c(c3cccnc3n1Cc1ccccc1)CCN(CC1CC1)C2]

100 mg (0.26 mmol) of 6-benzyl-2,3,4,6-tetrahydropyrido[3,4-c][1,8]naphthyridine-5-(1H)-one (7-1) was dissolved in 1 mL of N,N-dimethylformamide, and 50 μL (0.51 mmol) of (bromomethyl)cyclopropane and 140 μL (0.77 mmol) of N,N-diisopropylethylamine were added. The reaction was stirred at room temperature overnight. After the reaction was completed as monitored by TLC, 5 mL of water was added, and the mixture was extracted with ethyl acetate (5 mL×3). The organic phases were combined, washed with saturated brine, dried over anhydrous sodium sulfate, concentrated by rotary evaporation under reduced pressure to remove the solvent, and purified by silica gel column chromatography. The resulting crude product was purified by semi-preparative HPLC, and freeze-dried to give a white solid (68.8 mg, yield: 78%). ¹H NMR (300 MHz, Chloroform-d) δ 8.49 (dd, J=4.6, 1.5 Hz, 1H), 7.81 (dd, J=7.9, 1.5 Hz, 1H), 7.49-7.43 (m, 2H), 7.24-7.05 (m, 4H), 5.75 (s, 2H), 3.65 (s, 2H), 2.87 (t, J=4.6 Hz, 2H), 2.81 (t, J=4.7 Hz, 2H), 2.47 (d, J=6.6 Hz, 2H), 1.03-0.91 (m, 1H), 0.61-0.53 (m, 2H), 0.23-0.16 (m, 2H). ¹³C NMR (75 MHz, CDCl3) δ 161.41, 148.72, 148.07, 139.34, 137.88, 131.66, 128.48, 128.19, 127.70, 127.02, 118.03, 115.79, 63.36, 51.40, 49.26, 44.01, 25.55, 8.66, 4.10. MS(ESI) m/z: 346.2 [M+H]⁺, 368.1 [M+Na]⁺.

### Example 60

### Preparation of 6-benzyl-3-(cyclobutylmethyl)-2,3,4,6-tetrahydropyrido[3,4-c][1,8]naphthyridine-5-(1H)-one (I-2)

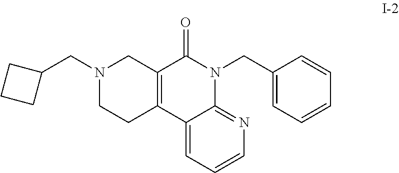 [SMILES: O=c1c2c(c3cccnc3n1Cc1ccccc1)CCN(CC1CCC1)C2]

The compound was prepared from compound 7-1 and (bromomethyl)cyclobutane by referring to the synthetic method for compound I-1 in Example 59. ¹H NMR (300 MHz, Chloroform-d) δ 8.54 (dd, J=4.6, 1.7 Hz, 1H), 7.90 (dd, J=7.9, 1.7 Hz, 1H), 7.47-7.40 (m, 2H), 7.26-7.13 (m, 4H), 5.77 (s, 2H), 3.54 (s, 2H), 2.92 (t, J=5.5 Hz, 2H), 2.76 (t, J=5.7 Hz, 2H), 2.71-2.60 (m, 3H), 2.18-2.06 (m, 2H), 1.96-1.71 (m, 4H). ¹³C NMR (75 MHz, CDCl₃) δ 161.50, 148.78, 148.19, 139.26, 137.87, 131.66, 128.47, 128.23, 127.81, 127.03, 118.03, 115.92, 64.84, 51.53, 49.49, 44.05, 33.77, 27.74, 25.52, 18.86. MS (ESI) m/z: 360.2 [M+H]⁺, 382.3 [M+Na]⁺.

### Example 61

### Preparation of 6-benzyl-3-(cyclopentylmethyl)-2,3,4,6-tetrahydropyrido[3,4-c][1,8]naphthyridine-5-(1H)-one (I-3)

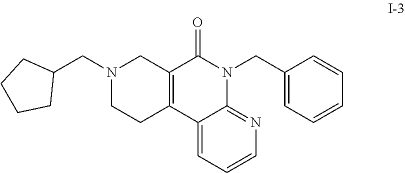 [SMILES: O=c1c2c(c3cccnc3n1Cc1ccccc1)CCN(CC1CCCC1)C2]

The compound was prepared from compound 7-1 and (bromomethyl)cyclopentane by referring to the synthetic method for compound I-1 in Example 59. ¹H NMR (300 MHz, Chloroform-d) δ 8.55 (dd, J=4.6, 1.7 Hz, 1H), 7.91 (dd, J=7.9, 1.7 Hz, 1H), 7.48-7.44 (m, 2H), 7.28-7.25 (m, 1H), 7.23-7.14 (m, 3H), 5.77 (s, 2H), 3.58 (s, 2H), 2.93 (t, J=5.6 Hz, 2H), 2.78 (t, J=5.7 Hz, 2H), 2.50 (d, J=7.4 Hz, 2H), 2.29-2.17 (m, 1H), 1.85-1.75 (m, 2H), 1.64-1.50 (m, 4H), 1.28-1.19 (m, 2H). ¹³C NMR (75 MHz, CDCl3) δ 161.57, 148.73, 148.20, 139.38, 137.91, 131.67, 128.57, 128.22, 127.96, 127.05, 118.02, 115.97, 64.32, 51.79, 49.65, 44.05, 37.31, 31.38, 25.61, 25.22. MS (ESI) m/z: 374.2 [M+H]⁺, 396.1 [M+Na]⁺.

### Example 62

### Preparation of 6-benzyl-3-(cyclohexylmethyl)-2,3,4,6-tetrahydropyrido[3,4-c][1,8]naphthyridine-5-(1H)-one (I-4)

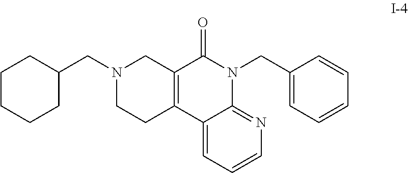 [SMILES: O=c1c2c(c3cccnc3n1Cc1ccccc1)CCN(CC1CCCCC1)C2]

The compound was prepared from compound 7-1 and (bromomethyl)cyclohexane by referring to the synthetic method for compound I-1 in Example 59. ¹H NMR (300 MHz, Chloroform-d) δ 8.55 (dd, J=4.6, 1.7 Hz, 1H), 7.92 (dd, J=7.9, 1.7 Hz, 1H), 7.49-7.41 (m, 2H), 7.28-7.15 (m, 4H), 5.77 (s, 2H), 3.53 (s, 2H), 2.93 (t, J=5.6 Hz, 2H), 2.74 (t, J=5.7 Hz, 2H), 2.37 (d, J=7.1 Hz, 2H), 1.84-1.59 (m, 7H), 1.29-1.23 (m, 2H), 1.00-0.83 (m, 2H). ¹³C NMR (75 MHz, CDCl3) δ 161.58, 148.73, 148.21, 139.41, 137.90, 131.67, 128.60, 128.22, 128.01, 127.06, 118.02, 115.99, 65.58, 58.44, 51.99, 49.82, 44.06, 35.16, 31.90, 26.80, 26.13, 25.65, 18.47. MS (ESI) m/z: 388.2 [M+H]⁺.

### Example 63

### Preparation of 3,6-dibenzyl-2,3,4,6-tetrahydropyrido[3,4-c][1,8]naphthyridine-5-(H)-one (I-5)

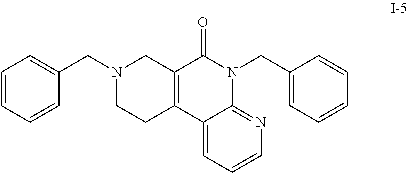 [SMILES: O=c1c2c(c3cccnc3n1Cc1ccccc1)CCN(Cc1ccccc1)C2]

The compound was prepared from compound 7-1 and benzyl bromide by referring to the synthetic method for compound I-1 in Example 59. ¹H NMR (300 MHz, Chloroform-d) δ 8.54 (dd, J=4.6, 1.7 Hz, 1H), 7.87 (dd, J=7.9, 1.7 Hz, 1H), 7.48-7.27 (m, 7H), 7.25-7.12 (m, 4H), 5.75 (s, 2H), 3.75 (s, 2H), 3.62 (s, 2H), 2.89 (t, J=5.4 Hz, 2H), 2.77 (t, J=5.6 Hz, 2H). ¹³C NMR (75 MHz, CDCl₃) δ 161.45, 148.82, 148.21, 139.42, 137.89, 137.78, 131.73, 129.23, 128.61, 128.44, 128.24, 127.84, 127.34, 127.09, 118.06, 115.92, 62.65, 51.74, 48.65, 44.08, 25.70. MS (ESI) m/z: 382.2 [M+H]⁺.

### Example 64

### Preparation of 6-benzyl-3-(2-fluorobenzyl)-2,3,4,6-tetrahydropyrido[3,4-c][1,8]naphthyridine-5-(1H)-one (I-6)

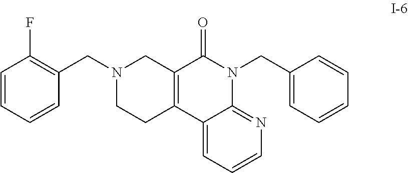 [SMILES: O=c1c2c(c3cccnc3n1Cc1ccccc1)CCN(Cc1ccccc1F)C2]

The compound was prepared from compound 7-1 and 2-fluorobenzyl bromide by referring to the synthetic method for compound I-1 in Example 59. MS (ESI) m/z: 400.2 [M+H]⁺.

### Example 65

### Preparation of 6-benzyl-3-(2-chlorobenzyl)-2,3,4,6-tetrahydropyrido[3,4-c][1,8]naphthyridine-5-(1H)-one (I-7)

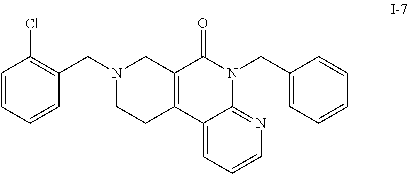 [SMILES: O=c1c2c(c3cccnc3n1Cc1ccccc1)CCN(Cc1ccccc1Cl)C2]

The compound was prepared from compound 7-1 and 2-chlorobenzyl bromide by referring to the synthetic method for compound I-1 in Example 59. ¹H NMR (300 MHz, Chloroform-d) δ 8.57 (dd, J=4.6, 1.5 Hz, 1H), 7.89 (dd, J=7.9, 1.5 Hz, 1H), 7.57-7.47 (m, 3H), 7.39 (dd, J=7.5, 1.6 Hz, 1H), 7.31-7.14 (m, 6H), 5.79 (s, 2H), 3.89 (s, 2H), 3.72 (s, 2H), 2.96-2.84 (m, 4H). ¹³C NMR (75 MHz, CDCl₃) δ 161.41, 148.83, 148.20, 139.38, 137.91, 135.62, 134.45, 131.72, 130.85, 129.60, 128.65, 128.45, 128.25, 127.76, 127.11, 126.81, 118.07, 115.88, 59.04, 51.64, 48.96, 44.09, 25.68; MS (ESI) m/z: 416.2 [M+H]⁺.

### Example 66

### Preparation of 6-benzyl-3-(2-bromobenzyl)-2,3,4,6-tetrahydropyrido[3,4-c][1,8]naphthyridine-5-(1H)-one (I-8)

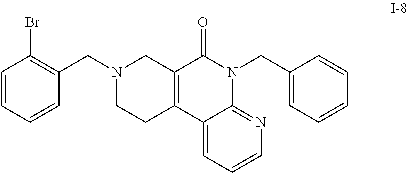 [SMILES: O=c1c2c(c3cccnc3n1Cc1ccccc1)CCN(Cc1ccccc1Br)C2]

The compound was prepared from compound 7-1 and 2-bromobenzyl bromide by referring to the synthetic method for compound I-1 in Example 59. MS (ESI) m/z: 460.1 [M+H]⁺.

### Example 67

### Preparation of 6-benzyl-3-(2-methylbenzyl)-2,3,4,6-tetrahydropyrido[3,4-c][1,8]naphthyridine-5-(1H)-one (I-9)

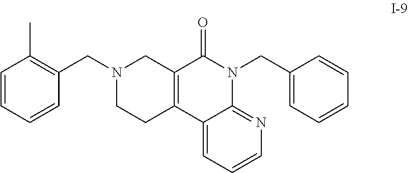 [SMILES: Cc1ccccc1CN1CCc2c(c(=O)n(Cc3ccccc3)c3ncccc23)C1]

The compound was prepared from compound 7-1 and 2-methylbenzyl bromide by referring to the synthetic method for compound I-1 in Example 59. MS (ESI) m/z: 396.2 [M+H]⁺.

### Example 68

### Preparation of 6-benzyl-3-(2-ethylbenzyl)-2,3,4,6-tetrahydropyrido[3,4-c][1,8]naphthyridine-5-(1H)-one (I-10)

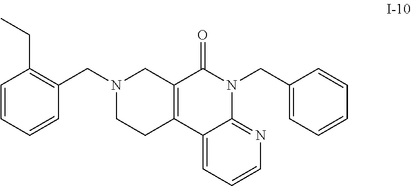 [SMILES: CCc1ccccc1CN1CCc2c(c(=O)n(Cc3ccccc3)c3ncccc23)C1]

The compound was prepared from compound 7-1 and 2-ethylbenzyl bromide by referring to the synthetic method for compound I-1 in Example 59. MS (ESI) m/z: 410.2 [M+H]⁺.

### Example 69

### Preparation of 6-benzyl-3-(2-methoxybenzyl)-2,3,4,6-tetrahydropyrido[3,4-c][1,8]naphthyridine-5-(1H)-one (I-11)

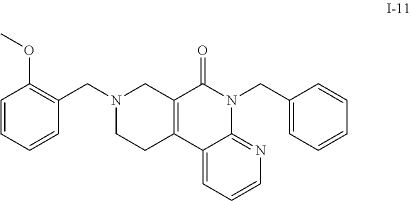 [SMILES: COc1ccccc1CN1CCc2c(c(=O)n(Cc3ccccc3)c3ncccc23)C1]

The compound was prepared from compound 7-1 and 2-methoxybenzyl bromide by referring to the synthetic method for compound I-1 in Example 59. MS (ESI) m/z: 412.2 [M+H]⁺.

### Example 70

### Preparation of 6-benzyl-3-(3-fluorobenzyl)-2,3,4,6-tetrahydropyrido[3,4-c][1,8]naphthyridine-5-(1H)-one (I-12)

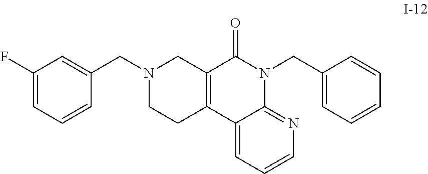 [SMILES: O=c1c2c(c3cccnc3n1Cc1ccccc1)CCN(Cc1cccc(F)c1)C2]

The compound was prepared from compound 7-1 and 2-fluorobenzyl bromide by referring to the synthetic method for compound I-1 in Example 59. MS (ESI) m/z: 400.2 [M+H]⁺.

### Example 71

### Preparation of 6-benzyl-3-(3-chlorobenzyl)-2,3,4,6-tetrahydropyrido[3,4-c][1,8]naphthyridine-5-(1H)-one (I-13)

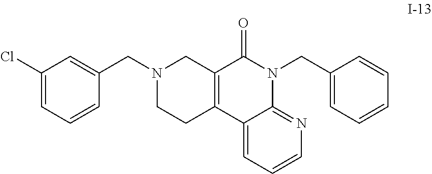 [SMILES: O=c1c2c(c3cccnc3n1Cc1ccccc1)CCN(Cc1cccc(Cl)c1)C2]

The compound was prepared from compound 7-1 and 3-chlorobenzyl bromide by referring to the synthetic method for compound I-1 in Example 59. ¹H NMR (300 MHz, Chloroform-d) δ 8.58 (dd, J=4.7, 1.7 Hz, 1H), 7.90 (dd, J=7.9, 1.7 Hz, 1H), 7.53-7.46 (m, 2H), 7.42 (s, 1H), 7.30-7.15 (m, 7H), 5.79 (s, 2H), 3.74 (s, 2H), 3.63 (s, 2H), 2.93 (t, J=5.5 Hz, 2H), 2.79 (t, J=5.6 Hz, 2H). ¹³C NMR (75 MHz, CDCl₃) δ 161.39, 148.89, 148.18, 140.12, 139.39, 137.86, 134.33, 131.75, 129.70, 129.03, 128.64, 128.25, 127.58, 127.52, 127.22, 127.12, 118.11, 115.84, 62.00, 51.64, 48.77, 44.09, 25.65; MS (ESI) m/z: 416.2 [M+H]⁺.

### Example 72

### Preparation of 6-benzyl-3-(3-bromobenzyl)-2,3,4,6-tetrahydropyrido[3,4-c][1,8]naphthyridine-5-(1H)-one (I-14)

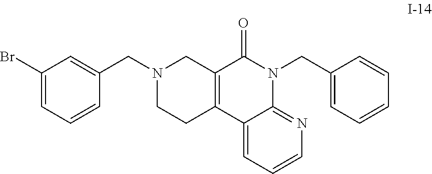 [SMILES: O=c1c2c(c3cccnc3n1Cc1ccccc1)CCN(Cc1cccc(Br)c1)C2]

The compound was prepared from compound 7-1 and 3-bromobenzyl bromide by referring to the synthetic method for compound I-1 in Example 59. MS (ESI) m/z: 460.1 [M+H]⁺.

### Example 73

### Preparation of 6-benzyl-3-(3-methylbenzyl)-2,3,4,6-tetrahydropyrido[3,4-c][1,8]naphthyridine-5-(1H)-one (I-15)

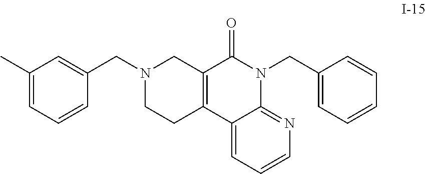 [SMILES: Cc1cccc(CN2CCc3c(c(=O)n(Cc4ccccc4)c4ncccc34)C2)c1]

The compound was prepared from compound 7-1 and 3-methylbenzyl bromide by referring to the synthetic method for compound I-1 in Example 59. MS (ESI) m/z: 396.2 [M+H]⁺.

### Example 74

### Preparation of 6-benzyl-3-(3-ethylbenzyl)-2,3,4,6-tetrahydropyrido[3,4-c][1,8]naphthyridine-5-(1H)-one (I-16)

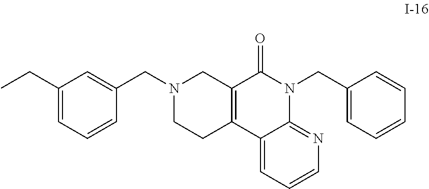 [SMILES: CCc1cccc(CN2CCc3c(c(=O)n(Cc4ccccc4)c4ncccc34)C2)c1]

The compound was prepared from compound 7-1 and 3-ethylbenzyl bromide by referring to the synthetic method for compound I-1 in Example 59. MS (ESI) m/z: 410.2 [M+H]⁺.

### Example 75

### Preparation of 6-benzyl-3-(3-methoxybenzyl)-2,3,4,6-tetrahydropyrido[3,4-c][1,8]naphthyridine-5-(1H)-one (I-17)

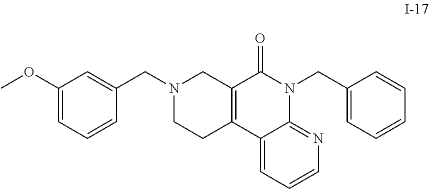 [SMILES: COc1cccc(CN2CCc3c(c(=O)n(Cc4ccccc4)c4ncccc34)C2)c1]

The compound was prepared from compound 7-1 and 3-methoxybenzyl bromide by referring to the synthetic method for compound I-1 in Example 59. ¹H NMR (300 MHz, Chloroform-d) δ 8.55 (dd, J=4.6, 1.7 Hz, 1H), 7.90 (dd, J=7.9, 1.7 Hz, 1H), 7.47-7.43 (m, 2H), 7.25-7.14 (m, 5H), 6.98-6.94 (m, 2H), 6.83-6.79 (m, 1H), 5.76 (s, 2H), 3.80 (s, 3H), 3.73 (s, 2H), 3.63 (s, 2H), 2.91 (t, J=5.5 Hz, 2H), 2.77 (t, J=5.6 Hz, 2H). ¹³C NMR (75 MHz, CDCl₃) δ 161.46, 159.74, 148.82, 148.22, 139.52, 139.41, 137.88, 131.72, 129.38, 128.60, 128.24, 127.87, 127.08, 121.50, 118.05, 115.93, 114.50, 112.84, 62.54, 55.25, 51.80, 48.58, 44.07, 25.70; MS (ESI) m/z: 412.2 [M+H]⁺.

### Example 76

### Preparation of 6-benzyl-3-(4-fluorobenzyl)-2,3,4,6-tetrahydropyrido[3,4-c][1,8]naphthyridine-5-(1H)-one (I-18)

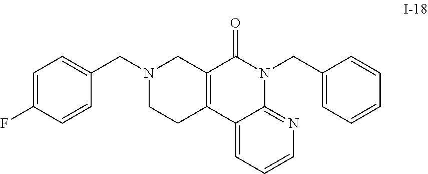 [SMILES: O=c1c2c(c3cccnc3n1Cc1ccccc1)CCN(Cc1ccc(F)cc1)C2]

The compound was prepared from compound 7-1 and 4-fluorobenzyl bromide by referring to the synthetic method for compound I-1 in Example 59. MS (ESI) m/z: 400.2 [M+H]⁺.

### Example 77

### Preparation of 6-benzyl-3-(4-chlorobenzyl)-2,3,4,6-tetrahydropyrido[3,4-c][1,8]naphthyridine-5-(1H)-one (I-19)

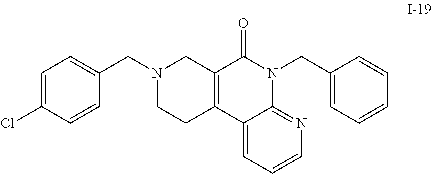 [SMILES: O=c1c2c(c3cccnc3n1Cc1ccccc1)CCN(Cc1ccc(Cl)cc1)C2]

The compound was prepared from compound 7-1 and 4-chlorobenzyl bromide by referring to the synthetic method for compound I-1 in Example 59. ¹H NMR (300 MHz, Chloroform-d) δ 8.50 (dd, J=4.6, 1.7 Hz, 1H), 7.78 (dd, J=7.9, 1.7 Hz, 1H), 7.49-7.41 (m, 2H), 7.29-7.05 (m, 8H), 5.72 (s, 2H), 3.65 (s, 2H), 3.55 (s, 2H), 2.80 (t, J=5.1 Hz, 2H), 2.70 (t, J=5.2 Hz, 2H). ¹³C NMR (75 MHz, CDCl₃) δ 161.36, 148.85, 148.13, 139.40, 137.91, 136.51, 132.96, 131.75, 130.45, 128.72, 128.56, 128.25, 127.57, 127.14, 118.11, 115.80, 61.84, 51.61, 48.76, 44.08, 25.64; MS (ESI) m/z: 416.2 [M+H]⁺.

### Example 78

### Preparation of 6-benzyl-3-(4-bromobenzyl)-2,3,4,6-tetrahydropyrido[3,4-c][1,8]naphthyridine-5-(1H)-one (I-20)

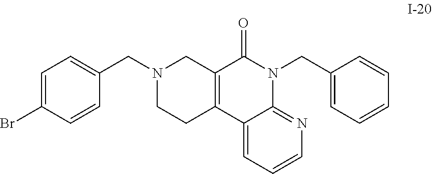 [SMILES: O=c1c2c(c3cccnc3n1Cc1ccccc1)CCN(Cc1ccc(Br)cc1)C2]

The compound was prepared from compound 7-1 and 4-bromobenzyl bromide by referring to the synthetic method for compound I-1 in Example 59. MS (ESI) m/z: 460.1 [M+H]⁺.

### Example 79

### Preparation of 6-benzyl-3-(4-methylbenzyl)-2,3,4,6-tetrahydropyrido[3,4-c][1,8]naphthyridine-5-(1H)-one (I-21)

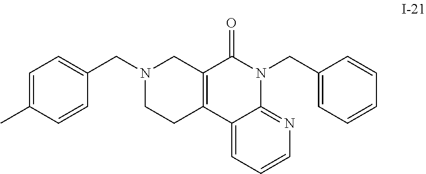 [SMILES: Cc1ccc(CN2CCc3c(c(=O)n(Cc4ccccc4)c4ncccc34)C2)cc1]

The compound was prepared from compound 7-1 and 4-methylbenzyl bromide by referring to the synthetic method for compound I-1 in Example 59. MS (ESI) m/z: 396.2 [M+H]⁺.

### Example 80

### Preparation of 6-benzyl-3-(4-ethylbenzyl)-2,3,4,6-tetrahydropyrido[3,4-c][1,8]naphthyridine-5-(1H)-one (I-22)

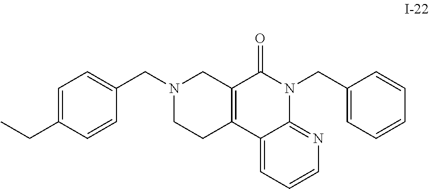 [SMILES: CCc1ccc(CN2CCc3c(c(=O)n(Cc4ccccc4)c4ncccc34)C2)cc1]

The compound was prepared from compound 7-1 and 4-ethylbenzyl bromide by referring to the synthetic method for compound I-1 in Example 59. MS (ESI) m/z: 410.2 [M+H]⁺.

### Example 81

### Preparation of 6-benzyl-3-(4-methoxybenzyl)-2,3,4,6-tetrahydropyrido[3,4-c][1,8]naphthyridine-5-(1H)-one (I-23)

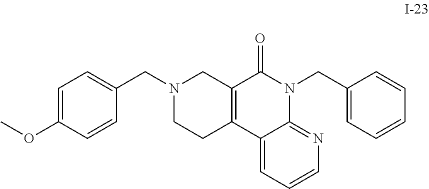 [SMILES: COc1ccc(CN2CCc3c(c(=O)n(Cc4ccccc4)c4ncccc34)C2)cc1]

The compound was prepared from compound 7-1 and 4-methoxybenzyl bromide by referring to the synthetic method for compound I-1 in Example 59. MS (ESI) m/z: 412.2 [M+H]⁺.

### Example 82

### 6-Benzyl-3-((1-methyl-1H-pyrazol-3-yl)methyl)-2,3,4,6-tetrahydropyrido[3,4-c][1,8]naphthyridine-5-(1H)-one (I-24)

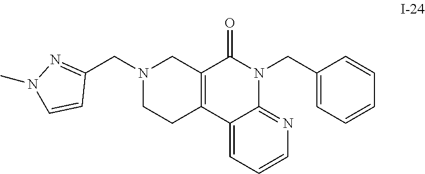 [SMILES: Cn1ccc(CN2CCc3c(c(=O)n(Cc4ccccc4)c4ncccc34)C2)n1]

The compound was prepared from compound 7-1 and 3-chloromethyl-1-methyl-1H-pyrazole by referring to the synthetic method for compound I-1 in Example 59. MS (ESI) m/z: 385.2 [M+H]⁺.

### Example 83

### Preparation of 6-benzyl-3-((1-methyl-1H-pyrazol-5-yl)methyl)-2,3,4,6-tetrahydropyrido[3,4-c][1,8]naphthyridine-5-(1H)-one (I-25)

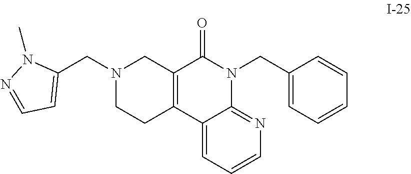 [SMILES: Cn1nccc1CN1CCc2c(c(=O)n(Cc3ccccc3)c3ncccc23)C1]

The compound was prepared from compound 7-1 and 5-chloromethyl-1-methyl-1H-pyrazole by referring to the synthetic method for compound I-1 in Example 59. MS (ESI) m/z: 386.2 [M+H]⁺.

### Example 84

### Preparation of 6-benzyl-3-((1,3-dimethyl-1H-pyrazol-5-yl)methyl)-2,3,4,6-tetrahydropyrido[3,4-c][1,8]naphthyridine-5-(1H)-one (I-26)

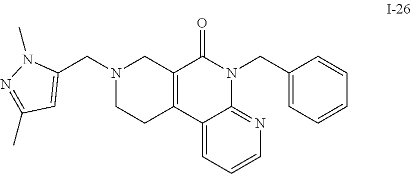 [SMILES: Cc1cc(CN2CCc3c(c(=O)n(Cc4ccccc4)c4ncccc34)C2)n(C)n1]

The compound was prepared from compound 7-1 and 5-chloromethyl-1,3-methyl-1H-pyrazole by referring to the synthetic method for compound I-1 in Example 59. MS (ESI) m/z: 400.2 [M+H]⁺.

### Example 85

### Preparation of 6-benzyl-3-(pyridin-2-ylmethyl)-2,3,4,6-tetrahydropyrido[3,4-c][1,8]naphthyridine-5-(1H)-one (I-27)

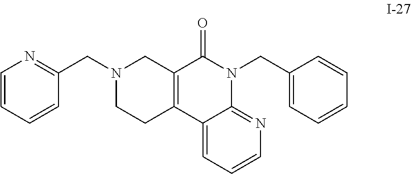 [SMILES: O=c1c2c(c3cccnc3n1Cc1ccccc1)CCN(Cc1ccccn1)C2]

The compound was prepared from compound 7-1 and 2-chloromethylpyridine by referring to the synthetic method for compound I-1 in Example 59. ¹H NMR (300 MHz, Methanol-d₄) δ 8.75-8.72 (m, 1H), 8.70 (dd, J=4.7, 1.7 Hz, 1H), 8.28 (dd, J=8.0, 1.7 Hz, 1H), 7.63-7.58 (m, 1H), 7.55-7.49 (m, 1H), 7.42 (dd, J=8.0, 4.7 Hz, 1H), 7.38-7.32 (m, 2H), 7.26-7.16 (m, 3H), 5.78 (s, 2H), 4.75 (s, 2H), 4.42 (s, 2H), 3.77 (t, J=6.2 Hz, 2H), 3.43 (t, J=6.1 Hz, 2H). MS (ESI) m/z: 383.2 [M+H]⁺.

### Preparation of 6-benzyl-3-(pyridin-3-ylmethyl)-2,3,4,6-tetrahydropyrido[3,4-c][1,8]naphthyridine-5-(1H)-one (I-28)

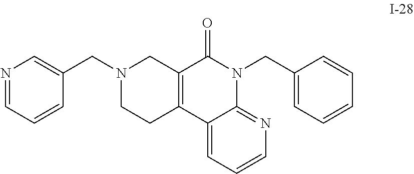 [SMILES: O=c1c2c(c3cccnc3n1Cc1ccccc1)CCN(Cc1cccnc1)C2]

The compound was prepared from compound 7-1 and 3-chloromethylpyridine by referring to the synthetic method for compound I-1 in Example 59. ¹H NMR (300 MHz, Methanol-d₄) δ 8.94-8.90 (m, 1H), 8.83-8.78 (m, 1H), 8.68 (dd, J=4.7, 1.7 Hz, 1H), 8.38 (dt, J=8.0, 1.8 Hz, 1H), 8.25 (dd, J=8.0, 1.7 Hz, 1H), 7.82 (dd, J=7.9, 5.3 Hz, 1H), 7.40 (dd, J=8.0, 4.7 Hz, 1H), 7.35-7.27 (m, 2H), 7.25-7.14 (m, 3H), 5.76 (s, 2H), 4.67 (s, 2H), 4.24 (s, 2H), 3.70 (t, J=6.1 Hz, 2H), 3.38 (t, J=6.1 Hz, 2H). MS (ESI) m/z: 383.2 [M+H]⁺.

### Example 87

### Preparation of 6-benzyl-3-(pyridin-4-ylmethyl)-2,3,4,6-tetrahydropyrido[3,4-c][1,8]naphthyridine-5-(1H)-one (I-29)

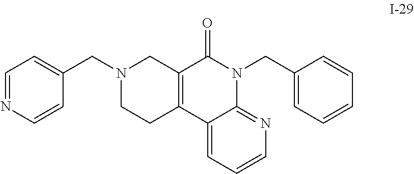 [SMILES: O=c1c2c(c3cccnc3n1Cc1ccccc1)CCN(Cc1ccncc1)C2]

The compound was prepared from compound 7-1 and 4-chloromethylpyridine by referring to the synthetic method for compound I-1 in Example 59. ¹H NMR (300 MHz, Methanol-d₄) δ 8.93-8.87 (m, 2H), 8.66 (dd, J=4.7, 1.6 Hz, 1H), 8.24 (dd, J=8.0, 1.7 Hz, 1H), 8.17-8.08 (m, 2H), 7.39 (dd, J=8.0, 4.7 Hz, 1H), 7.33-7.28 (m, 2H), 7.24-7.15 (m, 3H), 5.76 (s, 2H), 4.67 (s, 2H), 4.17 (s, 2H), 3.59 (t, J=6.0 Hz, 2H), 3.38-3.34 (m, 2H). MS (ESI) m/z: 383.2 [M+H]⁺.

### Example 88

### Preparation of 6-benzyl-3-((4-chloropyridin-2-yl)methyl)-2,3,4,6-tetrahydropyrido[3,4-c][1,8]naphthyridine-5-(1H)-one (I-30)

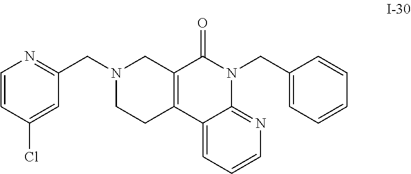 [SMILES: O=c1c2c(c3cccnc3n1Cc1ccccc1)CCN(Cc1cc(Cl)ccn1)C2]

The compound was prepared from compound 7-1 and 2-chloromethyl-4-chloropyridine by referring to the synthetic method for compound I-1 in Example 59. ¹H NMR (300 MHz, Methanol-d₄) δ 8.73 (dd, J=4.7, 1.7 Hz, 1H), 8.67 (d, J=5.4 Hz, 1H), 8.30 (dd, J=8.0, 1.7 Hz, 1H), 7.69 (d, J=1.6 Hz, 1H), 7.60 (dd, J=5.4, 2.0 Hz, 1H), 7.44 (dd, J=8.0, 4.7 Hz, 1H), 7.40-7.35 (m, 2H), 7.29-7.18 (m, 3H), 5.80 (s, 2H), 4.77 (s, 2H), 4.44 (s, 2H), 3.79 (t, J=6.1 Hz, 2H), 3.44 (t, J=6.2 Hz, 2H) MS (ESI) m/z: 417.2 [M+H]⁺.

### Example 89

### Preparation of 6-benzyl-3-((1-methyl-1H-imidazol-2-yl)methyl)-2,3,4,6-tetrahydropyrido[3,4-c][1,8]naphthyridine-5-(1H)-one (I-31)

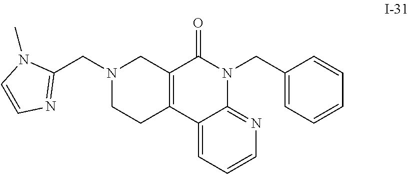 [SMILES: Cn1ccnc1CN1CCc2c(c(=O)n(Cc3ccccc3)c3ncccc23)C1]

The compound was prepared from compound 7-1 and 2-chloromethyl-1-methyl-1H-imidazole by referring to the synthetic method for compound I-1 in Example 59. MS (ESI) m/z: 386.2 [M+H]⁺.

### Example 90

### Preparation of 6-benzyl-3-((1-methyl-1H-imidazol-4-yl)methyl)-2,3,4,6-tetrahydropyrido[3,4-c][1,8]naphthyridine-5-(1H)-one (I-32)

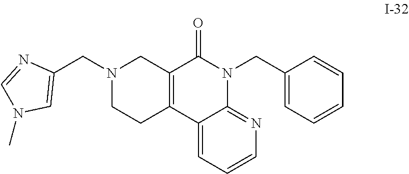 [SMILES: Cn1cnc(CN2CCc3c(c(=O)n(Cc4ccccc4)c4ncccc34)C2)c1]

The compound was prepared from compound 7-1 and 4-chloromethyl-1-methyl-1H-imidazole by referring to the synthetic method for compound I-1 in Example 59. MS (ESI) m/z: 386.2 [M+H]⁺.

### Example 91

### Preparation of 6-benzyl-3-((1-methyl-1H-imidazol-5-yl)methyl)-2,3,4,6-tetrahydropyrido[3,4-c][1,8]naphthyridine-5-(1H)-one (I-33)

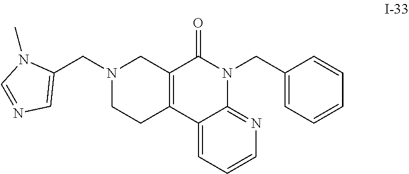 [SMILES: Cn1cncc1CN1CCc2c(c(=O)n(Cc3ccccc3)c3ncccc23)C1]

The compound was prepared from compound 7-1 and 5-chloromethyl-1-methyl-1H-imidazole by referring to the synthetic method for compound I-1 in Example 59. MS (ESI) m/z: 386.2 [M+H]⁺.

### Example 92

### Preparation of 6-benzyl-3-((3,5-dimethylisoxazol-4-yl)methyl)-2,3,4,6-tetrahydropyrido[3,4-c][1,8]naphthyridine-5-(1H)-one (I-34)

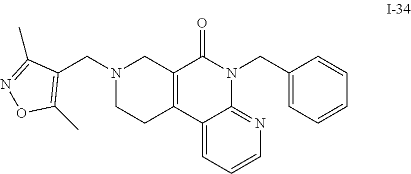 [SMILES: Cc1noc(C)c1CN1CCc2c(c(=O)n(Cc3ccccc3)c3ncccc23)C1]

The compound was prepared from compound 7-1 and 4-chloromethyl-3,5-dimethylisoxazole by referring to the synthetic method for compound I-1 in Example 59. MS (ESI) m/z: 401.2 [M+H]⁺.

### Example 93

### Preparation of 6-benzyl-3-((2-methylthiazol-5-yl)methyl)-2,3,4,6-tetrahydropyrido[3,4-c][1,8]naphthyridine-5-(1H)-one (I-35)

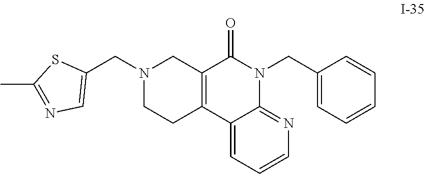 [SMILES: Cc1ncc(CN2CCc3c(c(=O)n(Cc4ccccc4)c4ncccc34)C2)s1]

The compound was prepared from compound 7-1 and 5-chloromethyl-2-methylthiazole by referring to the synthetic method for compound I-1 in Example 59. MS (ESI) m/z: 403.2 [M+H]⁺.

### Example 94

### Preparation of 3-benzyl-6-(cyclopropylmethyl)-2,3,4,6-tetrahydropyrido[3,4-c][1,8]naphthyridine-5-(1H)-one (I-36)

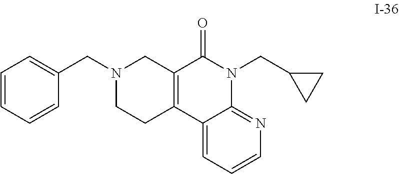 [SMILES: O=c1c2c(c3cccnc3n1CC1CC1)CCN(Cc1ccccc1)C2]

The Boc protecting group in compound 6-A-2 was first removed by referring to the method in Example 58, and then the compound was prepared by reacting the resulting amine with benzyl bromide by referring to the synthetic method for compound I-1 in Example 59. ¹H NMR (300 MHz, Chloroform-d) δ 8.54 (dd, J=4.6, 1.7 Hz, 1H), 7.91 (dd, J=7.9, 1.7 Hz, 1H), 7.41-7.28 (m, 5H), 7.16 (dd, J=7.9, 4.7 Hz, 1H), 4.43 (d, J=7.1 Hz, 2H), 3.77 (s, 2H), 3.63 (s, 2H), 2.92 (t, J=5.5 Hz, 2H), 2.79 (t, J=5.7 Hz, 2H), 1.44-1.35 (m, 1H), 0.55-0.48 (m, 2H), 0.46-0.38 (m, 2H). ¹³C NMR (75 MHz, CDCl₃) δ 161.61, 148.65, 148.39, 139.07, 137.81, 131.61, 129.21, 128.41, 127.78, 127.30, 117.75, 115.85, 62.61, 51.81, 48.61, 45.20, 25.64, 10.35, 3.96, 3.86; MS (ESI) m/z: 346.2 [M+H]⁺.

### Example 95

### Preparation of 3-benzyl-6-(cyclobutylmethyl)-2,3,4,6-tetrahydropyrido[3,4-c][1,8]naphthyridine-5-(1H)-one (I-37)

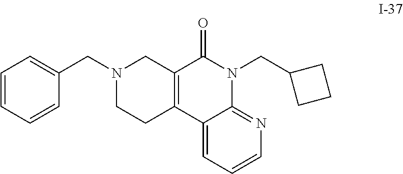 [SMILES: O=c1c2c(c3cccnc3n1CC1CCC1)CCN(Cc1ccccc1)C2]

The Boc protecting group in compound 6-A-3 was first removed by referring to the method in Example 58, and then the compound was prepared by reacting the resulting amine with benzyl bromide by referring to the synthetic method for compound I-1 in Example 59. ¹H NMR (300 MHz, Chloroform-d) δ 8.54 (dd, J=4.6, 1.7 Hz, 1H), 7.90 (dd, J=7.9, 1.7 Hz, 1H), 7.41-7.27 (m, 5H), 7.16 (dd, J=7.9, 4.7 Hz, 1H), 4.61 (d, J=7.3 Hz, 2H), 3.77 (s, 2H), 3.63 (s, 2H), 2.95-2.84 (m, 3H), 2.79 (t, J=5.7 Hz, 2H), 1.96-1.80 (m, 6H). ¹³C NMR (75 MHz, CDCl3) δ 161.70, 148.57, 148.49, 138.92, 137.81, 131.54, 129.24, 128.41, 127.75, 127.30, 117.70, 115.77, 62.64, 51.85, 48.60, 45.57, 34.80, 26.34, 25.65, 18.42; MS (ESI) m/z: 360.2 [M+H]⁺.

### Example 96

### Preparation of 3-benzyl-6-(cyclopentylmethyl)-2,3,4,6-tetrahydropyrido[3,4-c][1,8]naphthyridine-5-(1H)-one (I-38)

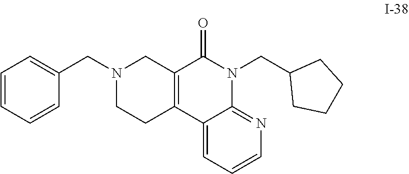 [SMILES: O=c1c2c(c3cccnc3n1CC1CCCC1)CCN(Cc1ccccc1)C2]

The Boc protecting group in compound 6-A-4 was first removed by referring to the method in Example 58, and then the compound was prepared by reacting the resulting amine with benzyl bromide by referring to the synthetic method for compound I-1 in Example 59. ¹H NMR (300 MHz, Chloroform-d) δ 8.54 (dd, J=4.6, 1.7 Hz, 1H), 7.89 (dd, J=7.9, 1.8 Hz, 1H), 7.41-7.23 (m, 5H), 7.15 (dd, J=7.9, 4.6 Hz, 1H), 4.52 (d, J=7.5 Hz, 2H), 3.77 (s, 2H), 3.63 (s, 2H), 2.92 (t, J=5.5 Hz, 2H), 2.79 (t, J=5.7 Hz, 2H), 2.53 (p, J=7.4 Hz, 1H), 1.72-1.56 (m, 4H), 1.53-1.34 (m, 4H). ¹³C NMR (75 MHz, CDCl₃) δ 161.75, 148.55, 138.93, 137.75, 131.53, 129.25, 128.41, 127.69, 127.31, 117.68, 115.77, 62.64, 51.81, 48.62, 45.21, 39.04, 30.31, 25.62, 24.93; MS (ESI) m/z: 374.2 [M+H]⁺.

### Example 97

### 3-Benzyl-6-(cyclohexylmethyl)-2,3,4,6-tetrahydropyrido[3,4-c][1,8]naphthyridine-5-(1H)-one (I-39)

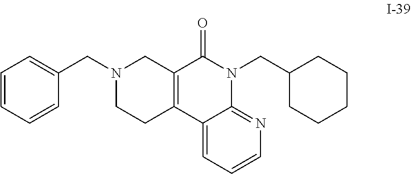 [SMILES: O=c1c2c(c3cccnc3n1CC1CCCCC1)CCN(Cc1ccccc1)C2]

The Boc protecting group in compound 6-A-5 was first removed by referring to the method in Example 58, and then the compound was prepared by reacting the resulting amine with benzyl bromide by referring to the synthetic method for compound I-1 in Example 59. ¹H NMR (300 MHz, Chloroform-d) δ 8.53 (dd, J=4.6, 1.7 Hz, 1H), 7.88 (dd, J=7.9, 1.7 Hz, 1H), 7.41-7.23 (m, 5H), 7.14 (dd, J=7.9, 4.6 Hz, 1H), 4.41 (d, J=7.3 Hz, 2H), 3.76 (s, 2H), 3.62 (s, 2H), 2.90 (t, J=5.3 Hz, 2H), 2.78 (t, J=5.6 Hz, 2H), 2.06-1.85 (m, 1H), 1.69-1.54 (m, 4H), 1.28-0.99 (m, 6H). ¹³C NMR (75 MHz, CDCl₃) δ 161.75, 148.63, 148.56, 138.97, 137.78, 131.52, 129.24, 128.40, 127.63, 127.29, 117.67, 115.69, 62.68, 51.79, 48.65, 46.75, 36.71, 30.87, 26.47, 25.98, 25.64; MS (ESI) m/z: 388.2 [M+H]⁺.

### Example 98

### 3-Benzyl-6-(2-fluorobenzyl)-2,3,4,6-tetrahydropyrido[3,4-c][1,8]naphthyridine-5-(1H)-one (I-40)

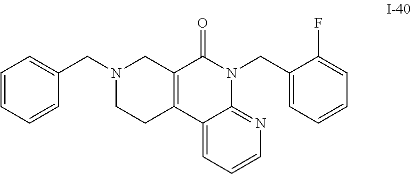 [SMILES: O=c1c2c(c3cccnc3n1Cc1ccccc1F)CCN(Cc1ccccc1)C2]

The Boc protecting group in compound 6-A-6 was first removed by referring to the method in Example 58, and then the compound was prepared by reacting the resulting amine with benzyl bromide by referring to the synthetic method for compound I-1 in Example 59. MS (ESI) m/z: 400.2 [M+H]⁺.

### Example 99

### 3-Benzyl-6-(2-chlorobenzyl)-2,3,4,6-tetrahydropyrido[3,4-c][1,8]naphthyridine-5-(1H)-one (I-41)

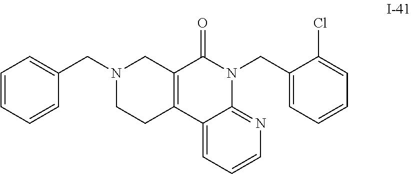 [SMILES: O=c1c2c(c3cccnc3n1Cc1ccccc1Cl)CCN(Cc1ccccc1)C2]

The Boc protecting group in compound 6-A-7 was first removed by referring to the method in Example 58, and then the compound was prepared by reacting the resulting amine with benzyl bromide by referring to the synthetic method for compound I-1 in Example 59. MS (ESI) m/z: 416.2 [M+H]⁺.

### Example 100

### 3-Benzyl-6-(2-bromobenzyl)-2,3,4,6-tetrahydropyrido[3,4-c][1,8]naphthyridine-5-(1H)-one (I-42)

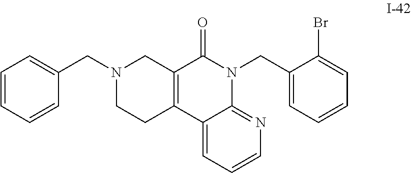 [SMILES: O=c1c2c(c3cccnc3n1Cc1ccccc1Br)CCN(Cc1ccccc1)C2]

The Boc protecting group in compound 6-A-8 was first removed by referring to the method in Example 58, and then the compound was prepared by reacting the resulting amine with benzyl bromide by referring to the synthetic method for compound I-1 in Example 59. MS (ESI) m/z: 460.1 [M+H]⁺.

### Example 101

### 3-Benzyl-6-(2-methylbenzyl)-2,3,4,6-tetrahydropyrido[3,4-c][1,8]naphthyridine-5-(1H)-one (I-43)

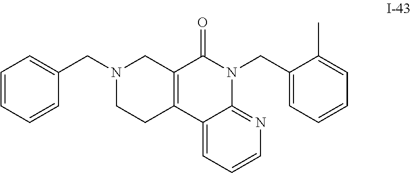 [SMILES: Cc1ccccc1Cn1c(=O)c2c(c3cccnc31)CCN(Cc1ccccc1)C2]

The Boc protecting group in compound 6-A-9 was first removed by referring to the method in Example 58, and then the compound was prepared by reacting the resulting amine with benzyl bromide by referring to the synthetic method for compound I-1 in Example 59. ¹H NMR (300 MHz, Chloroform-d) δ 8.48 (dd, J=4.6, 1.7 Hz, 1H), 7.94 (dd, J=7.9, 1.7 Hz, 1H), 7.41-7.25 (m, 5H), 7.20-7.13 (m, 2H), 7.07 (t, J=6.9 Hz, 1H), 6.95 (t, J=7.5 Hz, 1H), 6.64 (d, J=7.6 Hz, 1H), 5.72 (s, 2H), 3.77 (s, 2H), 3.65 (s, 2H), 2.97 (t, J=5.6 Hz, 2H), 2.82 (t, J=5.7 Hz, 2H), 2.50 (s, 3H). ¹³C NMR (75 MHz, CDCl₃) δ 161.48, 149.07, 148.34, 139.56, 137.68, 135.57, 135.47, 131.75, 130.10, 129.28, 128.44, 127.80, 127.36, 126.57, 125.87, 125.12, 118.10, 115.85, 62.67, 51.75, 48.63, 41.99, 25.75, 19.45; MS (ESI) m/z: 396.2 [M+H]⁺.

### Example 102

### 3-Benzyl-6-(2-ethylbenzyl)-2,3,4,6-tetrahydropyrido[3,4-c][1,8]naphthyridine-5-(H)-one (I-44)

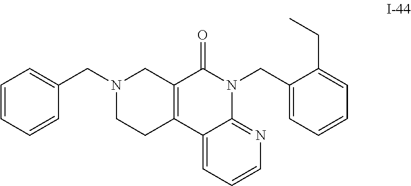 [SMILES: CCc1ccccc1Cn1c(=O)c2c(c3cccnc31)CCN(Cc1ccccc1)C2]

The Boc protecting group in compound 6-A-10 was first removed by referring to the method in Example 58, and then the compound was prepared by reacting the resulting amine with benzyl bromide by referring to the synthetic method for compound I-1 in Example 59. MS (ESI) m/z: 410.2 [M+H]⁺.

### Example 103

### Preparation of 3-benzyl-6-(2-methoxybenzyl)-2,3,4,6-tetrahydropyrido[3,4-c][1,8]naphthyridine-5-(1H)-one (I-45)

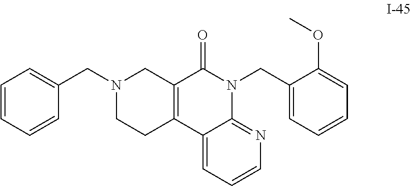 [SMILES: COc1ccccc1Cn1c(=O)c2c(c3cccnc31)CCN(Cc1ccccc1)C2]

The Boc protecting group in compound 6-A-11 was first removed by referring to the method in Example 58, and then the compound was prepared by reacting the resulting amine with benzyl bromide by referring to the synthetic method for compound I-1 in Example 59. MS (ESI) m/z: 412.2 [M+H]⁺.

### Example 104

### 3-Benzyl-6-(3-fluorobenzyl)-2,3,4,6-tetrahydropyrido[3,4-c][1,8]naphthyridine-5-(1H)-one (I-46)

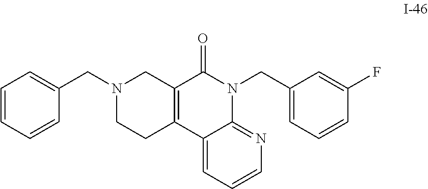 [SMILES: O=c1c2c(c3cccnc3n1Cc1cccc(F)c1)CCN(Cc1ccccc1)C2]

The Boc protecting group in compound 6-A-12 was first removed by referring to the method in Example 58, and then the compound was prepared by reacting the resulting amine with benzyl bromide by referring to the synthetic method for compound I-1 in Example 59. MS (ESI) m/z: 400.2 [M+H]⁺.

### Example 105

### 3-Benzyl-6-(3-chlorobenzyl)-2,3,4,6-tetrahydropyrido[3,4-c][1,8]naphthyridine-5-(1H)-one (I-47)

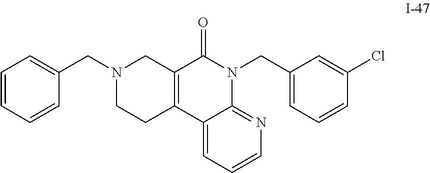 [SMILES: O=c1c2c(c3cccnc3n1Cc1cccc(Cl)c1)CCN(Cc1ccccc1)C2]

The Boc protecting group in compound 6-A-13 was first removed by referring to the method in Example 58, and then the compound was prepared by reacting the resulting amine with benzyl bromide by referring to the synthetic method for compound I-1 in Example 59. MS (ESI) m/z: 416.2 [M+H]⁺.

### Example 106

### 3-Benzyl-6-(3-bromobenzyl)-2,3,4,6-tetrahydropyrido[3,4-c][1,8]naphthyridine-5-(1H)-one (I-48)

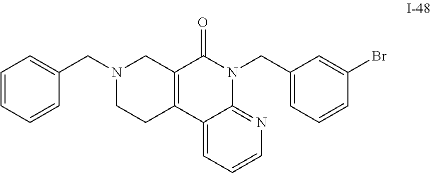 [SMILES: O=c1c2c(c3cccnc3n1Cc1cccc(Br)c1)CCN(Cc1ccccc1)C2]

The Boc protecting group in compound 6-A-14 was first removed by referring to the method in Example 58, and then the compound was prepared by reacting the resulting amine with benzyl bromide by referring to the synthetic method for compound I-1 in Example 59. MS (ESI) m/z: 460.1 [M+H]⁺.

### Example 107

### 3-Benzyl-6-(3-methylbenzyl)-2,3,4,6-tetrahydropyrido[3,4-c][1,8]naphthyridine-5-(1H)-one (I-49)

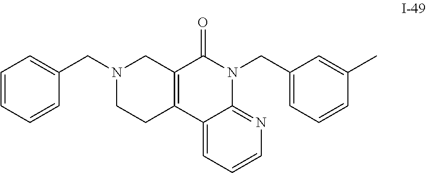 [SMILES: Cc1cccc(Cn2c(=O)c3c(c4cccnc42)CCN(Cc2ccccc2)C3)c1]

The Boc protecting group in compound 6-A-15 was first removed by referring to the method in Example 58, and then the compound was prepared by reacting the resulting amine with benzyl bromide by referring to the synthetic method for compound I-1 in Example 59. MS (ESI) m/z: 396.2 [M+H]⁺.

### Example 108

### 3-Benzyl-6-(3-ethylbenzyl)-2,3,4,6-tetrahydropyrido[3,4-c][1,8]naphthyridine-5-(l)-one (I-50)

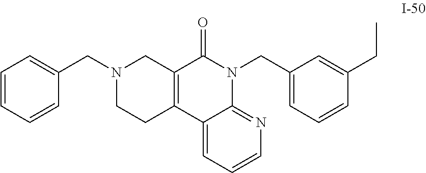 [SMILES: CCc1cccc(Cn2c(=O)c3c(c4cccnc42)CCN(Cc2ccccc2)C3)c1]

The Boc protecting group in compound 6-A-16 was first removed by referring to the method in Example 58, and then the compound was prepared by reacting the resulting amine with benzyl bromide by referring to the synthetic method for compound I-1 in Example 59. MS (ESI) m/z: 410.2 [M+H]⁺.

### Example 109

### Preparation of 3-benzyl-6-(3-methoxybenzyl)-2,3,4,6-tetrahydropyrido[3,4-c][1,8]naphthyridine-5-(1H)-one (I-51)

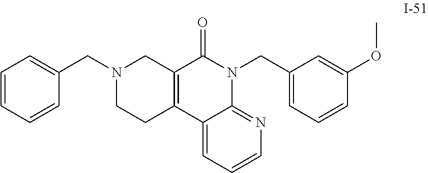 [SMILES: COc1cccc(Cn2c(=O)c3c(c4cccnc42)CCN(Cc2ccccc2)C3)c1]

The Boc protecting group in compound 6-A-17 was first removed by referring to the method in Example 58, and then the compound was prepared by reacting the resulting amine with benzyl bromide by referring to the synthetic method for compound I-1 in Example 59. MS (ESI) m/z: 412.2 [M+H]⁺.

### Example 110

### 3-Benzyl-6-(4-fluorobenzyl)-2,3,4,6-tetrahydropyrido[3,4-c][1,8]naphthyridine-5-(1H)-one (I-52)

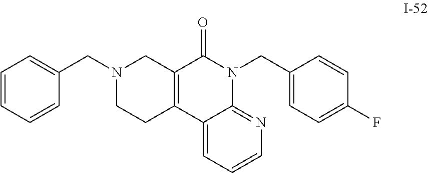 [SMILES: O=c1c2c(c3cccnc3n1Cc1ccc(F)cc1)CCN(Cc1ccccc1)C2]

The Boc protecting group in compound 6-A-18 was first removed by referring to the method in Example 58, and then the compound was prepared by reacting the resulting amine with benzyl bromide by referring to the synthetic method for compound I-1 in Example 59. MS (ESI) m/z: 400.2 [M+H]⁺.

### Example 111

### 3-Benzyl-6-(4-chlorobenzyl)-2,3,4,6-tetrahydropyrido[3,4-c][1,8]naphthyridine-5-(1H)-one (I-53)

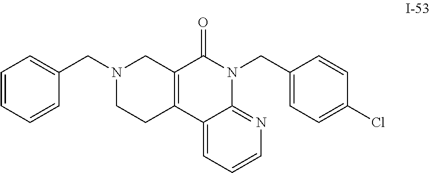 [SMILES: O=c1c2c(c3cccnc3n1Cc1ccc(Cl)cc1)CCN(Cc1ccccc1)C2]

The Boc protecting group in compound 6-A-19 was first removed by referring to the method in Example 58, and then the compound was prepared by reacting the resulting amine with benzyl bromide by referring to the synthetic method for compound I-1 in Example 59. MS (ESI) m/z: 416.2 [M+H]⁺.

### Example 112

### 3-Benzyl-6-(4-bromobenzyl)-2,3,4,6-tetrahydropyrido[3,4-c][1,8]naphthyridine-5-(1H)-one (I-54)

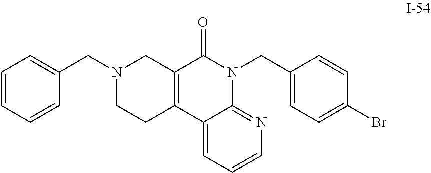 [SMILES: O=c1c2c(c3cccnc3n1Cc1ccc(Br)cc1)CCN(Cc1ccccc1)C2]

The Boc protecting group in compound 6-A-20 was first removed by referring to the method in Example 58, and then the compound was prepared by reacting the resulting amine with benzyl bromide by referring to the synthetic method for compound I-1 in Example 59. MS (ESI) m/z: 460.1 [M+H]⁺.

### Example 113

### 3-Benzyl-6-(4-methylbenzyl)-2,3,4,6-tetrahydropyrido[3,4-c][1,8]naphthyridine-5-(1H)-one (I-55)

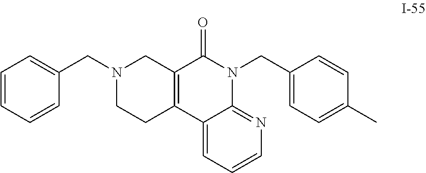 [SMILES: Cc1ccc(Cn2c(=O)c3c(c4cccnc42)CCN(Cc2ccccc2)C3)cc1]

The Boc protecting group in compound 6-A-21 was first removed by referring to the method in Example 58, and then the compound was prepared by reacting the resulting amine with benzyl bromide by referring to the synthetic method for compound I-1 in Example 59. MS (ESI) m/z: 396.2 [M+H]⁺.

### Example 114

### 3-Benzyl-6-(4-ethylbenzyl)-2,3,4,6-tetrahydropyrido[3,4-c][1,8]naphthyridine-5-(l)-one (I-56)

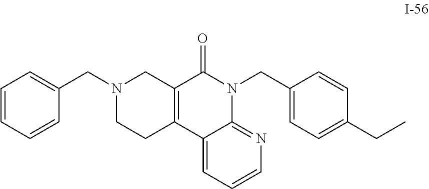 [SMILES: CCc1ccc(Cn2c(=O)c3c(c4cccnc42)CCN(Cc2ccccc2)C3)cc1]

The Boc protecting group in compound 6-A-22 was first removed by referring to the method in Example 58, and then the compound was prepared by reacting the resulting amine with benzyl bromide by referring to the synthetic method for compound I-1 in Example 59. MS (ESI) m/z: 410.2 [M+H]⁺.

### Example 115

### Preparation of 3-benzyl-6-(4-methoxybenzyl)-2,3,4,6-tetrahydropyrido[3,4-c][1,8]naphthyridine-5-(1H)-one (I-57)

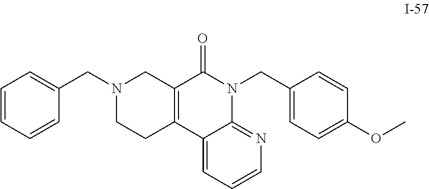 [SMILES: COc1ccc(Cn2c(=O)c3c(c4cccnc42)CCN(Cc2ccccc2)C3)cc1]

The Boc protecting group in compound 6-A-23 was first removed by referring to the method in Example 58, and then the compound was prepared by reacting the resulting amine with benzyl bromide by referring to the synthetic method for compound I-1 in Example 59. MS (ESI) m/z: 412.2 [M+H]⁺.

### Example 116

### 3-Benzyl-6-((1-methyl-1H-pyrazol-3-yl)methyl)-2,3,4,6-tetrahydropyrido[3,4-c][1,8]naphthyridine-5-(1H)-one (I-58)

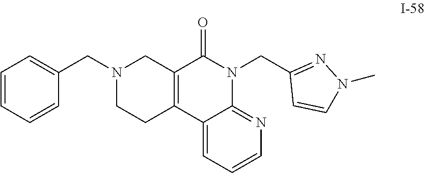 [SMILES: Cn1ccc(Cn2c(=O)c3c(c4cccnc42)CCN(Cc2ccccc2)C3)n1]

The Boc protecting group in compound 6-A-25 was first removed by referring to the method in Example 58, and then the compound was prepared by reacting the resulting amine with benzyl bromide by referring to the synthetic method for compound I-1 in Example 59. MS (ESI) m/z: 386.2 [M+H]⁺.

### Example 117

### 3-Benzyl-6-((1-methyl-1H-pyrazol-5-yl)methyl)-2,3,4,6-tetrahydropyrido[3,4-c][1,8]naphthyridine-5-(1H)-one (I-59)

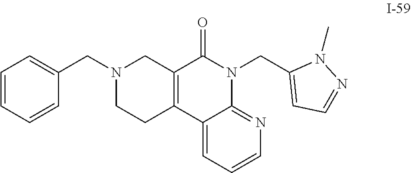 [SMILES: Cn1nccc1Cn1c(=O)c2c(c3cccnc31)CCN(Cc1ccccc1)C2]

The Boc protecting group in compound 6-A-26 was first removed by referring to the method in Example 58, and then the compound was prepared by reacting the resulting amine with benzyl bromide by referring to the synthetic method for compound I-1 in Example 59. MS (ESI) m/z: 386.2 [M+H]⁺.

### Example 118

### 3-Benzyl-6-((1,3-dimethyl-1H-pyrazol-5-yl)methyl)-2,3,4,6-tetrahydropyrido[3,4-c][1,8]naphthyridine-5-(1H)-one (I-60)

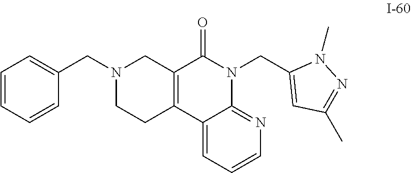 [SMILES: Cc1cc(Cn2c(=O)c3c(c4cccnc42)CCN(Cc2ccccc2)C3)n(C)n1]

The Boc protecting group in compound 6-A-27 was first removed by referring to the method in Example 58, and then the compound was prepared by reacting the resulting amine with benzyl bromide by referring to the synthetic method for compound I-1 in Example 59. MS (ESI) m/z: 400.1 [M+H]⁺.

### Example 119

### 3-Benzyl-6-(pyridin-2-ylmethyl)-2,3,4,6-tetrahydropyrido[3,4-c][1,8]naphthyridine-5-(1H)-one (I-61)

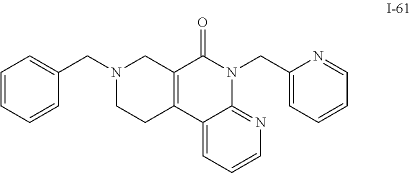 [SMILES: O=c1c2c(c3cccnc3n1Cc1ccccn1)CCN(Cc1ccccc1)C2]

The Boc protecting group in compound 6-A-28 was first removed by referring to the method in Example 58, and then the compound was prepared by reacting the resulting amine with benzyl bromide by referring to the synthetic method for compound I-1 in Example 59. ¹H NMR (300 MHz, Methanol-d₄) δ 8.71-8.66 (m, 1H), 8.60 (dd, J=4.7, 1.6 Hz, 1H), 8.37-8.26 (m, 2H), 7.85-7.74 (m, 2H), 7.65-7.58 (m, 2H), 7.56-7.48 (m, 3H), 7.42 (dd, J=8.0, 4.7 Hz, 1H), 6.03 (s, 2H), 4.61 (s, 2H), 4.27 (s, 2H), 3.75 (s, 2H), 3.41 (t, J=5.7 Hz, 2H); MS (ESI) m/z: 383.2 [M+H]⁺.

### Example 120

### 3-Benzyl-6-(pyridin-3-ylmethyl)-2,3,4,6-tetrahydropyrido[3,4-c][1,8]naphthyridine-5-(1H)-one (I-62)

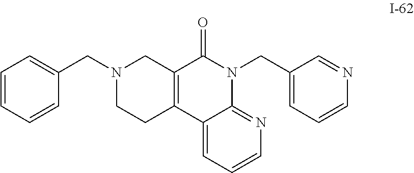 [SMILES: O=c1c2c(c3cccnc3n1Cc1cccnc1)CCN(Cc1ccccc1)C2]

The Boc protecting group in compound 6-A-29 was first removed by referring to the method in Example 58, and then the compound was prepared by reacting the resulting amine with benzyl bromide by referring to the synthetic method for compound I-1 in Example 59. ¹H NMR (300 MHz, Methanol-d₄) δ 9.01 (s, 1H), 8.75 (d, J=5.6 Hz, 1H), 8.72-8.64 (m, 2H), 8.27 (dd, J 10=8.0, 1.5 Hz, 1H), 8.00 (dd, J=8.0, 5.8 Hz, 1H), 7.66-7.57 (m, 2H), 7.53-7.47 (m, 3H), 7.42 (dd, J=8.0, 4.7 Hz, 1H), 5.92 (s, 2H), 4.62 (s, 2H), 4.29 (s, 2H), 3.75 (s, 2H), 3.41 (t, J=5.2 Hz, 2H); MS (ESI) m/z: 383.2 [M+H]⁺.

### Example 121

### 3-Benzyl-6-(pyridin-4-ylmethyl)-2,3,4,6-tetrahydropyrido[3,4-c][1,8]naphthyridine-5-(1H)-one (I-63)

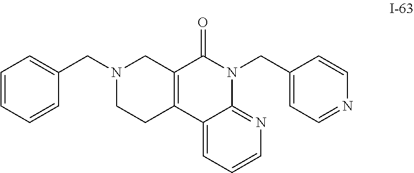 [SMILES: O=c1c2c(c3cccnc3n1Cc1ccncc1)CCN(Cc1ccccc1)C2]

The Boc protecting group in compound 6-A-30 was first removed by referring to the method in Example 58, and then the compound was prepared by reacting the resulting amine with benzyl bromide by referring to the synthetic method for compound I-1 in Example 59. ¹H NMR (300 MHz, Methanol-d₄) δ 8.74 (d, J=6.7 Hz, 2H), 8.58 (dd, J=4.7, 1.5 Hz, 1H), 8.30 (dd, J=8.0, 1.6 Hz, 1H), 7.96 (d, J=6.7 Hz, 2H), 7.66-7.58 (m, 2H), 7.54-7.48 (m, 3H), 7.42 (dd, J=8.0, 4.7 Hz, 1H), 6.00 (s, 2H), 4.62 (s, 2H), 4.30 (s, 2H), 3.77 (s, 2H), 3.44 (t, J=5.6 Hz, 2H); MS (ESI) m/z: 383.2 [M+H]⁺.

### Example 122

### 3-Benzyl-6-((4-chloropyridin-2-yl)methyl)-2,3,4,6-tetrahydropyrido[3,4-c][1,8]naphthyridine-5-(1H)-one (I-64)

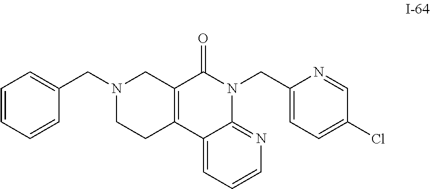 [SMILES: O=c1c2c(c3cccnc3n1Cc1ccc(Cl)cn1)CCN(Cc1ccccc1)C2]

The Boc protecting group in compound 6-A-31 was first removed by referring to the method in Example 58, and then the compound was prepared by reacting the resulting amine with benzyl bromide by referring to the synthetic method for compound I-1 in Example 59. ¹H NMR (300 MHz, Methanol-d₄) δ 8.56 (dd, J=4.7, 1.6 Hz, 1H), 8.43-8.32 (m, 1H), 8.26 (dd, J=8.0, 1.6 Hz, 1H), 7.66-7.58 (m, 2H), 7.54-7.47 (m, 3H), 7.45-7.41 (m, 2H), 7.38 (dd, J=8.0, 4.7 Hz, 1H), 5.88 (s, 2H), 4.62 (s, 2H), 4.31 (s, 2H), 3.77 (s, 2H), 3.43 (t, J=5.3 Hz, 2H); MS (ESI) m/z: 417.1 [M+H]⁺.

### Example 123

### 3-Benzyl-6-((1-methyl-1H-imidazol-2-yl)methyl)-2,3,4,6-tetrahydropyrido[3,4-c][1,8]naphthyridine-5-(1H)-one (I-65)

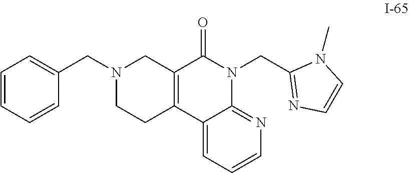 [SMILES: Cn1ccnc1Cn1c(=O)c2c(c3cccnc31)CCN(Cc1ccccc1)C2]

The Boc protecting group in compound 6-A-32 was first removed by referring to the method in Example 58, and then the compound was prepared by reacting the resulting amine with benzyl bromide by referring to the synthetic method for compound I-1 in Example 59. MS (ESI) m/z: 386.2 [M+H]⁺.

### Example 124

### 3-Benzyl-6-((1-methyl-1H-imidazol-4-yl)methyl)-2,3,4,6-tetrahydropyrido[3,4-c][1,8]naphthyridine-5-(1H)-one (I-66)

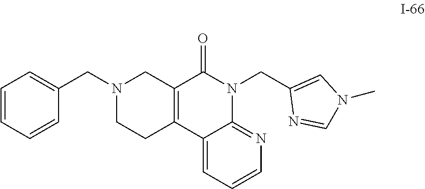 [SMILES: Cn1cnc(Cn2c(=O)c3c(c4cccnc42)CCN(Cc2ccccc2)C3)c1]

The Boc protecting group in compound 6-A-33 was first removed by referring to the method in Example 58, and then the compound was prepared by reacting the resulting amine with benzyl bromide by referring to the synthetic method for compound I-1 in Example 59. MS (ESI) m/z: 386.2 [M+H]⁺.

### Example 125

### 3-Benzyl-6-((1-methyl-1H-imidazol-5-yl)methyl)-2,3,4,6-tetrahydropyrido[3,4-c][1,8]naphthyridine-5-(1H)-one (I-67)

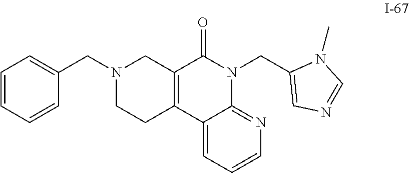 [SMILES: Cn1cncc1Cn1c(=O)c2c(c3cccnc31)CCN(Cc1ccccc1)C2]

The Boc protecting group in compound 6-A-34 was first removed by referring to the method in Example 58, and then the compound was prepared by reacting the resulting amine with benzyl bromide by referring to the synthetic method for compound I-1 in Example 59. MS (ESI) m/z: 386.2 [M+H]⁺.

### Example 126

### 3-Benzyl-6-((3,5-dimethylisoxazol-4-yl)methyl)-2,3,4,6-tetrahydropyrido[3,4-c][1,8]naphthyridine-5-(1H)-one (I-68)

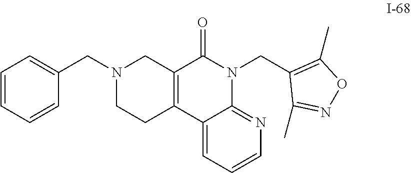 [SMILES: Cc1noc(C)c1Cn1c(=O)c2c(c3cccnc31)CCN(Cc1ccccc1)C2]

The Boc protecting group in compound 6-A-35 was first removed by referring to the method in Example 58, and then the compound was prepared by reacting the resulting amine with benzyl bromide by referring to the synthetic method for compound I-1 in Example 59. MS (ESI) m/z: 401.2 [M+H]⁺.

### Example 127

### 3-Benzyl-6-((2-methylthiazol-5-yl)methyl)-2,3,4,6-tetrahydropyrido[3,4-c][1,8]naphthyridine-5-(1H)-one (I-69)

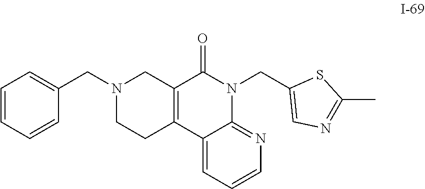 [SMILES: Cc1ncc(Cn2c(=O)c3c(c4cccnc42)CCN(Cc2ccccc2)C3)s1]

The Boc protecting group in compound 6-A-36 was first removed by referring to the method in Example 58, and then the compound was prepared by reacting the resulting amine with benzyl bromide by referring to the synthetic method for compound I-1 in Example 59. MS (ESI) m/z: 403.2 [M+H]⁺.

### Example 128

### 3-Benzyl-6-(4-(trifluoromethyl)benzyl)-2,3,4,6-tetrahydropyrido[3,4-c][1,8]naphthyridine-5-(1H)-one (I-70)

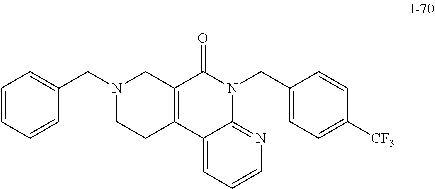 [SMILES: O=c1c2c(c3cccnc3n1Cc1ccc(C(F)(F)F)cc1)CCN(Cc1ccccc1)C2]

The Boc protecting group in compound 6-A-24 was first removed by referring to the method in Example 58, and then the compound was prepared by reacting the resulting amine with benzyl bromide by referring to the synthetic method for compound I-1 in Example 59. ¹H NMR (300 MHz, Chloroform-d) δ 8.57 (dd, J=4.7, 1.7 Hz, 1H), 7.96 (dd, J=7.9, 1.7 Hz, 1H), 7.61-7.49 (m, 4H), 7.44-7.27 (m, 5H), 7.23 (dd, J=7.9, 4.7 Hz, 1H), 5.82 (s, 2H), 3.79 (s, 2H), 3.66 (s, 2H), 2.97 (t, J=5.4 Hz, 2H), 2.83 (t, J=5.6 Hz, 2H). ¹³C NMR (75 MHz, CDCl₃) δ 161.40, 148.88, 148.02, 141.88, 139.78, 137.70, 131.91, 129.44, 129.22, 129.01, 128.76, 128.45, 127.84, 127.37, 125.23, 118.33, 115.99, 62.64, 51.65, 48.62, 43.75, 25.75; MS (ESI) m/z: 450.2 [M+H]⁺.

### Example 129

### 3-(3-Fluorobenzyl)-6-(4-fluorobenzyl)-2,3,4,6-tetrahydropyrido[3,4-c][1,8]naphthyridine-5-(1H)-one (I-71)

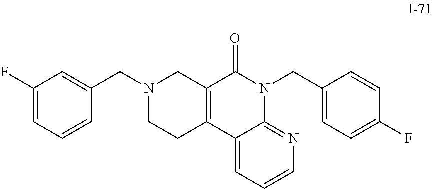 [SMILES: O=c1c2c(c3cccnc3n1Cc1ccc(F)cc1)CCN(Cc1cccc(F)c1)C2]

The Boc protecting group in compound 6-A-18 was first removed by referring to the method in Example 58, and then the compound was prepared by reacting the resulting amine with 3-fluorobenzyl bromide by referring to the synthetic method for compound I-1 in Example 59. ¹H NMR (300 MHz, Chloroform-d) δ 8.56 (dd, J=4.6, 1.6 Hz, 1H), 7.90 (dd, J=7.9, 1.6 Hz, 1H), 7.52-7.45 (m, 2H), 7.31-7.24 (m, 1H), 7.20-7.09 (m, 3H), 6.97-6.87 (m, 3H), 5.70 (s, 2H), 3.73 (s, 2H), 3.60 (s, 2H), 2.90 (t, J=5.3 Hz, 2H), 2.78 (t, J=5.6 Hz, 2H). ¹³C NMR (75 MHz, CDCl₃) δ 164.64, 163.58, 161.35, 148.84, 148.06, 140.71, 139.48, 133.70, 131.82, 130.74, 129.91, 127.64, 124.59, 118.18, 115.90, 115.58, 115.12, 114.84, 114.35, 114.07, 62.01, 51.59, 48.75, 43.33, 25.66; MS (ESI) m/z: 418.2 [M+H]⁺.

### Example 130

### 3-(3-Chlorobenzyl)-6-(4-fluorobenzyl)-2,3,4,6-tetrahydropyrido[3,4-c][1,8]naphthyridine-5-(1H)-one (I-72)

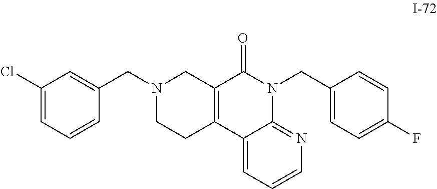 [SMILES: O=c1c2c(c3cccnc3n1Cc1ccc(F)cc1)CCN(Cc1cccc(Cl)c1)C2]

The Boc protecting group in compound 6-A-18 was first removed by referring to the method in Example 58, and then the compound was prepared by reacting the resulting amine with 3-chlorobenzyl bromide by referring to the synthetic method for compound I-1 in Example 59. ¹H NMR (300 MHz, Chloroform-d) δ 8.57 (dd, J=4.6, 1.6 Hz, 1H), 7.91 (dd, J=7.9, 1.7 Hz, 1H), 7.52-7.46 (m, 2H), 7.39 (s, 1H), 7.26-7.22 (m, 3H), 7.19 (dd, J=7.9, 4.7 Hz, 1H), 6.94-6.88 (m, 2H), 5.70 (s, 2H), 3.72 (s, 2H), 3.60 (s, 2H), 2.92 (t, J=5.4 Hz, 2H), 2.78 (t, J=5.6 Hz, 2H). 13C NMR (75 MHz, Chloroform-d) δ 163.59, 161.34, 160.34, 148.85, 148.08, 140.08, 139.44, 134.34, 133.67, 131.81, 130.72, 129.68, 129.01, 127.63, 127.52, 127.18, 118.17, 115.90, 115.13, 114.84, 61.98, 51.60, 48.74, 43.34, 25.66; MS (ESI) m/z: 434.1 [M+H]⁺.

### Example 131

### 3-(3-Bromobenzyl)-6-(4-fluorobenzyl)-2,3,4,6-tetrahydropyrido[3,4-c][1,8]naphthyridine-5-(1H)-one (I-73)

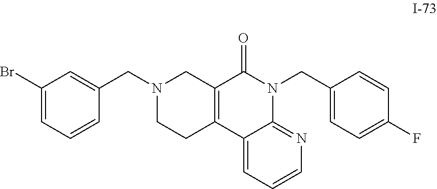 [SMILES: O=c1c2c(c3cccnc3n1Cc1ccc(F)cc1)CCN(Cc1cccc(Br)c1)C2]

The Boc protecting group in compound 6-A-18 was first removed by referring to the method in Example 58, and then the compound was prepared by reacting the resulting amine with 3-bromobenzyl bromide by referring to the synthetic method for compound I-1 in Example 59. ¹H NMR (300 MHz, Chloroform-d) δ 8.56 (dd, J=4.6, 1.6 Hz, 1H), 7.90 (dd, J=7.9, 1.7 Hz, 1H), 7.55-7.46 (m, 3H), 7.40-7.36 (m, 1H), 7.31-7.27 (m, 1H), 7.21-7.15 (m, 2H), 6.94-6.88 (m, 2H), 5.70 (s, 2H), 3.70 (s, 2H), 3.59 (s, 2H), 2.91 (t, J=5.4 Hz, 2H), 2.77 (t, J=5.6 Hz, 2H). 13C NMR (75 MHz, Chloroform-d) δ 163.58, 161.33, 160.33, 148.85, 148.07, 140.41, 139.45, 133.64, 131.90, 131.82, 130.73, 130.63, 130.44, 129.99, 127.65, 122.63, 118.17, 115.88, 115.12, 114.84, 61.93, 51.59, 48.74, 43.34, 25.65; MS (ESI) m/z: 478.1 [M+H]⁺.

### Example 132

### 3-(3-Fluorobenzyl)-6-(4-chlorobenzyl)-2,3,4,6-tetrahydropyrido[3,4-c][1,8]naphthyridine-5-(1H)-one (I-74)

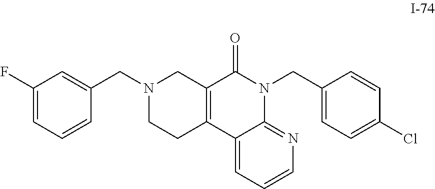 [SMILES: O=c1c2c(c3cccnc3n1Cc1ccc(Cl)cc1)CCN(Cc1cccc(F)c1)C2]

The Boc protecting group in compound 6-A-19 was first removed by referring to the method in Example 58, and then the compound was prepared by reacting the resulting amine with 3-fluorobenzyl bromide by referring to the synthetic method for compound I-1 in Example 59. ¹H NMR (300 MHz, Chloroform-d) δ 8.55 (dd, J=4.6, 1.6 Hz, 1H), 7.92 (dd, J=7.9, 1.6 Hz, 1H), 7.44-7.39 (m, 2H), 7.29-7.24 (m, 1H), 7.21-7.09 (m, 5H), 6.99-6.92 (m, 1H), 5.70 (s, 2H), 3.74 (s, 2H), 3.60 (s, 2H), 2.92 (t, J=5.3 Hz, 2H), 2.78 (t, J=5.6 Hz, 2H). ¹³C NMR (75 MHz, Chloroform-d) δ 164.65, 161.34, 148.86, 148.05, 140.71, 139.53, 136.39, 132.88, 131.83, 130.46, 130.25, 129.90, 129.79, 128.34, 127.68, 124.58, 118.21, 115.92, 114.36, 114.08, 62.02, 51.60, 48.74, 43.45, 25.70; MS (ESI) m/z: 434.1 [M+H]⁺.

### Example 133

### 3-(3-Chlorobenzyl)-6-(4-chlorobenzyl)-2,3,4,6-tetrahydropyrido[3,4-c][1,8]naphthyridine-5-(1H)-one (I-75)

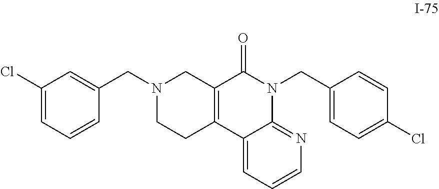 [SMILES: O=c1c2c(c3cccnc3n1Cc1ccc(Cl)cc1)CCN(Cc1cccc(Cl)c1)C2]

The Boc protecting group in compound 6-A-19 was first removed by referring to the method in Example 58, and then the compound was prepared by reacting the resulting amine with 3-chlorobenzyl bromide by referring to the synthetic method for compound I-1 in Example 59. ¹H NMR (300 MHz, Chloroform-d) δ 8.56 (dd, J=4.6, 1.6 Hz, 1H), 7.93 (dd, J=7.9, 1.7 Hz, 1H), 7.44-7.38 (m, 3H), 7.26-7.17 (m, 6H), 5.70 (s, 2H), 3.72 (s, 2H), 3.60 (s, 2H), 2.94 (t, J=5.5 Hz, 2H), 2.79 (t, J=5.7 Hz, 2H). ¹³C NMR (75 MHz, CDCl₃) δ 161.34, 148.87, 148.06, 140.09, 139.51, 136.37, 134.35, 132.89, 131.83, 130.23, 129.68, 129.01, 128.35, 127.66, 127.52, 127.17, 118.21, 115.91, 61.98, 51.60, 48.73, 43.46, 25.69; MS (ESI) m/z: 450.1 [M+H]⁺.

### Example 134

### 3-(3-Bromobenzyl)-6-(4-chlorobenzyl)-2,3,4,6-tetrahydropyrido[3,4-c][1,8]naphthyridine-5-(1H)-one (I-76)

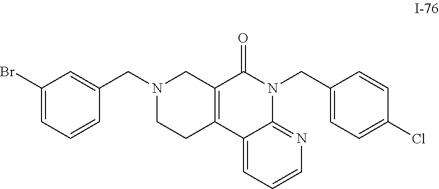 [SMILES: O=c1c2c(c3cccnc3n1Cc1ccc(Cl)cc1)CCN(Cc1cccc(Br)c1)C2]

The Boc protecting group in compound 6-A-19 was first removed by referring to the method in Example 58, and then the compound was prepared by reacting the resulting amine with 3-bromobenzyl bromide by referring to the synthetic method for compound I-1 in Example 59. ¹H NMR (300 MHz, Chloroform-d) δ 8.54 (dd, J=4.6, 1.5 Hz, 1H), 7.89 (dd, J=7.9, 1.6 Hz, 1H), 7.54 (s, 1H), 7.43-7.36 (m, 3H), 7.31-7.26 (m, 1H), 7.21-7.14 (m, 4H), 5.69 (s, 2H), 3.70 (s, 2H), 3.58 (s, 2H), 2.89 (t, J=5.2 Hz, 2H), 2.76 (t, J=5.6 Hz, 2H). ¹³C NMR (75 MHz, Chloroform-d) δ 161.31, 148.87, 148.02, 140.43, 139.54, 136.39, 132.87, 131.90, 131.84, 130.44, 130.26, 130.00, 128.34, 127.65, 127.60, 122.64, 118.23, 115.88, 61.93, 51.60, 48.74, 43.45, 25.68; MS (ESI) m/z: 494.1 [M+H]⁺.

### Example 135

### 3-(3-Fluorobenzyl)-6-(4-bromobenzyl)-2,3,4,6-tetrahydropyrido[3,4-c][1,8]naphthyridine-5-(1H)-one (I-77)

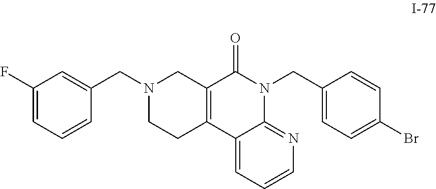 [SMILES: O=c1c2c(c3cccnc3n1Cc1ccc(Br)cc1)CCN(Cc1cccc(F)c1)C2]

The Boc protecting group in compound 6-A-20 was first removed by referring to the method in Example 58, and then the compound was prepared by reacting the resulting amine with 3-fluorobenzyl bromide by referring to the synthetic method for compound I-1 in Example 59. ¹H NMR (300 MHz, Chloroform-d) δ 8.55 (dd, J=4.6, 1.6 Hz, 1H), 7.92 (dd, J=7.9, 1.6 Hz, 1H), 7.35 (s, 4H), 7.27-7.09 (m, 4H), 6.99-6.92 (m, 1H), 5.69 (s, 2H), 3.74 (s, 2H), 3.60 (s, 2H), 2.92 (t, J=5.3 Hz, 2H), 2.79 (t, J=5.6 Hz, 2H); ¹³C NMR (75 MHz, Chloroform-d) δ 164.65, 161.34, 148.86, 148.05, 140.69, 139.53, 136.89, 131.83, 131.30, 130.59, 129.90, 127.69, 124.57, 124.54, 121.06, 118.22, 115.92, 115.58, 114.37, 114.09, 62.00, 51.60, 48.73, 43.51, 25.70; MS (ESI) m/z: 478.1 [M+H]⁺.

### Example 136

### 3-(3-Chlorobenzyl)-6-(4-bromobenzyl)-2,3,4,6-tetrahydropyrido[3,4-c][1,8]naphthyridine-5-(1H)-one (I-78)

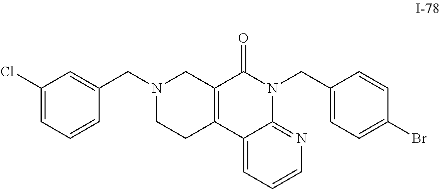 [SMILES: O=c1c2c(c3cccnc3n1Cc1ccc(Br)cc1)CCN(Cc1cccc(Cl)c1)C2]

The Boc protecting group in compound 6-A-20 was first removed by referring to the method in Example 58, and then the compound was prepared by reacting the resulting amine with 3-chlorobenzyl bromide by referring to the synthetic method for compound I-1 in Example 59. ¹H NMR (300 MHz, Chloroform-d) δ 8.55 (dd, J=4.6, 1.6 Hz, 1H), 7.92 (dd, J=7.9, 1.6 Hz, 1H), 7.40-7.37 (m, 1H), 7.35 (s, 4H), 7.26-7.23 (m, 3H), 7.19 (dd, J=7.9, 4.7 Hz, 1H), 5.68 (s, 2H), 3.72 (s, 2H), 3.59 (s, 2H), 2.92 (t, J=5.3 Hz, 2H), 2.78 (t, J=5.6 Hz, 2H). ¹³C NMR (75 MHz, Chloroform-d) δ 161.33, 148.87, 148.03, 140.09, 139.54, 136.89, 134.35, 131.84, 131.30, 130.59, 129.68, 129.01, 127.63, 127.52, 127.18, 121.06, 118.23, 115.90, 61.98, 51.59, 48.73, 43.51, 25.69; MS (ESI) m/z: 494.1 [M+H]⁺.

### Example 137

### 3-(3-Bromobenzyl)-6-(4-bromobenzyl)-2,3,4,6-tetrahydropyrido[3,4-c][1,8]naphthyridine-5-(1H)-one (I-79)

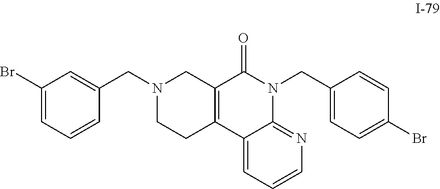 [SMILES: O=c1c2c(c3cccnc3n1Cc1ccc(Br)cc1)CCN(Cc1cccc(Br)c1)C2]

The Boc protecting group in compound 6-A-20 was first removed by referring to the method in Example 58, and then the compound was prepared by reacting the resulting amine with 3-bromobenzyl bromide by referring to the synthetic method for compound I-1 in Example 59. ¹H NMR (300 MHz, Chloroform-d) δ 8.54 (dd, J=4.6, 1.6 Hz, 1H), 7.89 (dd, J=7.9, 1.6 Hz, 1H), 7.54 (s, 1H), 7.41-7.28 (m, 6H), 7.20-7.14 (m, 2H), 5.67 (s, 2H), 3.70 (s, 2H), 3.58 (s, 2H), 2.90 (t, J=5.3 Hz, 2H), 2.76 (t, J=5.5 Hz, 2H); ¹³C NMR (75 MHz, Chloroform-d) δ 161.30, 148.89, 148.00, 140.39, 139.57, 136.90, 131.90, 131.86, 131.30, 130.60, 130.45, 130.01, 127.67, 127.56, 122.64, 121.06, 118.26, 115.87, 61.92, 51.58, 48.74, 43.52, 25.67; MS (ESI) m/z: 538.0 [M+H]⁺.

### Example 138

### 3-Benzyl-6-(4-(trifluoromethyl)benzyl)-2,3,4,6-tetrahydrobenzo[c][2,7]naphthyridine-5-(1H)-one (I-80)

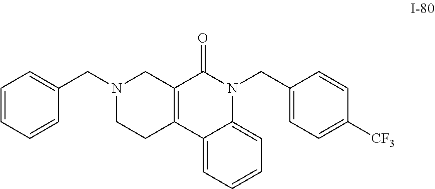 [SMILES: O=c1c2c(c3ccccc3n1Cc1ccc(C(F)(F)F)cc1)CCN(Cc1ccccc1)C2]

The Boc protecting group in compound 6-B-1 was first removed by referring to the method in Example 58, and then the compound was prepared by reacting the resulting amine with benzyl bromide by referring to the synthetic method for compound I-1 in Example 59. MS (ESI) m/z: 449.2 [M+H]⁺.

### 8-Benzyl-5-(4-(trifluoromethyl)benzyl)-7,8,9,10-tetrahydropyrimidinyl[4,5-c][2,7]naphthyridine-6-(5H)-one (I-81)

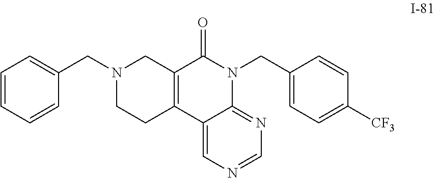 [SMILES: O=c1c2c(c3cncnc3n1Cc1ccc(C(F)(F)F)cc1)CCN(Cc1ccccc1)C2]

The Boc protecting group in compound 6-C-1 was first removed by referring to the method in Example 58, and then the compound was prepared by reacting the resulting amine with benzyl bromide by referring to the synthetic method for compound I-1 in Example 59. MS (ESI) m/z: 451.2 [M+H]⁺.

### Example 140

### 6-Benzyl-3-(3-cyanobenzyl)-2,3,4,6-tetrahydropyrido[3,4-c][1,8]naphthyridine-5-(1H)-one (I-82)

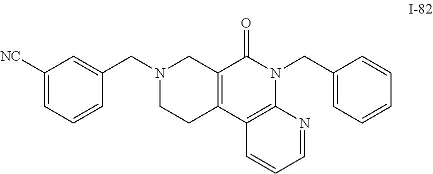 [SMILES: N#Cc1cccc(CN2CCc3c(c(=O)n(Cc4ccccc4)c4ncccc34)C2)c1]

The compound was prepared by reacting compound 7-1 with 3-cyanobenzyl bromide by referring to the synthetic method for compound I-1 in Example 59. MS (ESI) m/z: 407.3 [M+H]⁺.

### Example 141

### 6-(4-Trifluoromethylbenzyl)-3-(3-cyanobenzyl)-2,3,4,6-tetrahydropyrido[3,4-c][1,8]naphthyridine-5-(1H)-one (I-83)

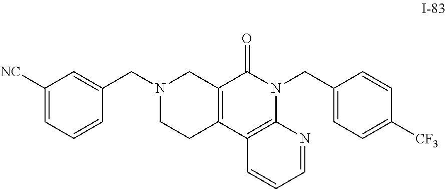 [SMILES: N#Cc1cccc(CN2CCc3c(c(=O)n(Cc4ccc(C(F)(F)F)cc4)c4ncccc34)C2)c1]

The Boc protecting group in compound 6-A-24 was first removed by referring to the method in Example 58, and then the compound was prepared by reacting the resulting amine with 3-cyanobenzyl bromide by referring to the synthetic method for compound I-1 in Example 59. MS (ESI) m/z: 475.3 [M+H]⁺.

### Example 142

### 6-(4-Fluorobenzyl)-3-(3-cyanobenzyl)-2,3,4,6-tetrahydropyrido[3,4-c][1,8]naphthyridine-5-(1H)-one (I-84)

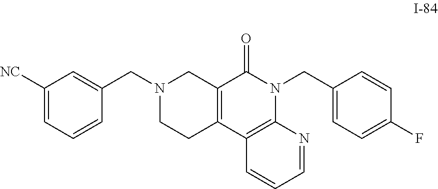 [SMILES: N#Cc1cccc(CN2CCc3c(c(=O)n(Cc4ccc(F)cc4)c4ncccc34)C2)c1]

The Boc protecting group in compound 6-A-18 was first removed by referring to the method in Example 58, and then the compound was prepared by reacting the resulting amine with 3-cyanobenzyl bromide by referring to the synthetic method for compound I-1 in Example 59. MS (ESI) m/z: 425.3 [M+H]⁺.

### Example 143

### 6-(4-Chlorobenzyl)-3-(3-cyanobenzyl)-2,3,4,6-tetrahydropyrido[3,4-c][1,8]naphthyridine-5-(1H)-one (I-85)

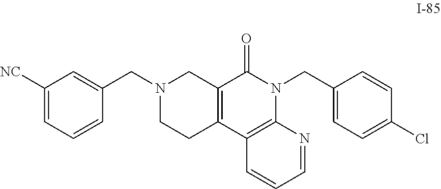 [SMILES: N#Cc1cccc(CN2CCc3c(c(=O)n(Cc4ccc(Cl)cc4)c4ncccc34)C2)c1]

The Boc protecting group in compound 6-A-19 was first removed by referring to the method in Example 58, and then the compound was prepared by reacting the resulting amine with 3-cyanobenzyl bromide by referring to the synthetic method for compound I-1 in Example 59. MS (ESI) m/z: 441.3 [M+H]⁺.

### 6-(4-Bromobenzyl)-3-(3-cyanobenzyl)-2,3,4,6-tetrahydropyrido[3,4-c][1,8]naphthyridine-5-(1H)-one (I-86)

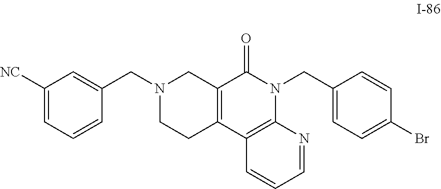 [SMILES: N#Cc1cccc(CN2CCc3c(c(=O)n(Cc4ccc(Br)cc4)c4ncccc34)C2)c1]

The Boc protecting group in compound 6-A-20 was first removed by referring to the method in Example 58, and then the compound was prepared by reacting the resulting amine with 3-cyanobenzyl bromide by referring to the synthetic method for compound I-1 in Example 59. MS (ESI) m/z: 485.2 [M+H]⁺.

### Example 145

### 6-(4-Methoxybenzyl)-3-(3-cyanobenzyl)-2,3,4,6-tetrahydropyrido[3,4-c][1,8]naphthyridine-5-(1H)-one (I-87)

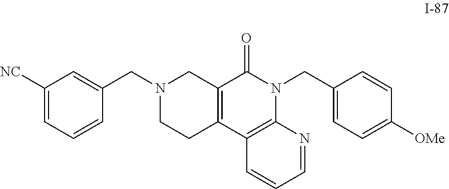 [SMILES: COc1ccc(Cn2c(=O)c3c(c4cccnc42)CCN(Cc2cccc(C#N)c2)C3)cc1]

The Boc protecting group in compound 6-A-23 was first removed by referring to the method in Example 58, and then the compound was prepared by reacting the resulting amine with 3-cyanobenzyl bromide by referring to the synthetic method for compound I-1 in Example 59. MS (ESI) m/z: 437.3 [M+H]⁺.

### Example 146

### 6-(4-Nitrobenzyl)-3-(3-cyanobenzyl)-2,3,4,6-tetrahydropyrido[3,4-c][1,8]naphthyridine-5-(1H)-one (I-88)

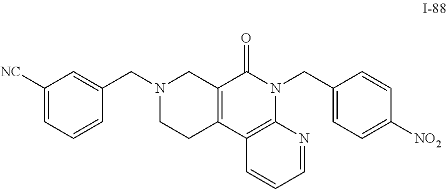 [SMILES: N#Cc1cccc(CN2CCc3c(c(=O)n(Cc4ccc([N+](=O)[O-])cc4)c4ncccc34)C2)c1]

The Boc protecting group in compound 6-A-37 was first removed by referring to the method in Example 58, and then the compound was prepared by reacting the resulting amine with 3-cyanobenzyl bromide by referring to the synthetic method for compound I-1 in Example 59. MS (ESI) m/z: 452.3 [M+H]⁺.

### Example 147

### 6-(4-Methylaminobenzyl)-3-(3-cyanobenzyl)-2,3,4,6-tetrahydropyrido[3,4-c][1,8]naphthyridine-5-(1H)-one (I-89) and 6-(4-dimethylaminobenzyl)-3-(3-cyanobenzyl)-2,3,4,6-tetrahydropyrido[3,4-c][1,8]naphthyridine-5-(H)-one (I-90)

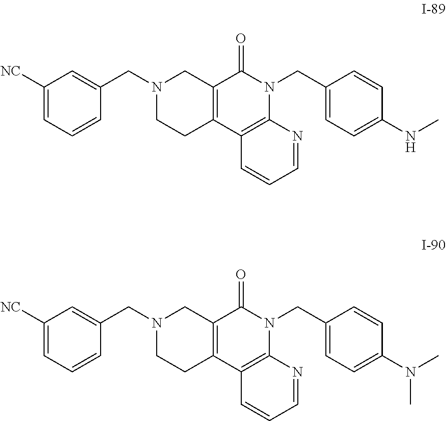 [SMILES: CNc1ccc(Cn2c(=O)c3c(c4cccnc42)CCN(Cc2cccc(C#N)c2)C3)cc1 ; CN(C)c1ccc(Cn2c(=O)c3c(c4cccnc42)CCN(Cc2cccc(C#N)c2)C3)cc1]

100 mg of I-88 was dissolved in methanol, and 0.1 mL of aqueous formaldehyde solution was added, followed by the addition of 20 mg of Pd—C (10%) under nitrogen atmosphere. The mixture was stirred under hydrogen atmosphere (1 atm) for 3 h. The reaction mixture was filtered through celite to remove the catalyst. The mixture containing I-89 and I-90 which remained after concentration of the filtrate was separated by semi-preparative HPLC to give pure compounds I-89 and 1-90. I-89: MS (ESI) m/z: 436.3 [M+H]⁺; I-90: MS (ESI) m/z: 450.3 [M+H]⁺.

### Example 148

### 6-(4-Methylbenzyl)-3-(3-cyanobenzyl)-2,3,4,6-tetrahydropyrido[3,4-c][1,8]naphthyridine-5-(1H)-one (I-91)

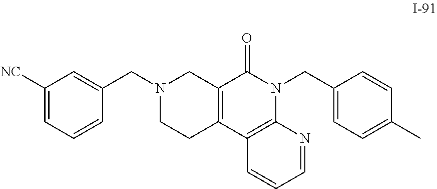 [SMILES: Cc1ccc(Cn2c(=O)c3c(c4cccnc42)CCN(Cc2cccc(C#N)c2)C3)cc1]

The Boc protecting group in compound 6-A-21 was first removed by referring to the method in Example 58, and then the compound was prepared by reacting the resulting amine with 3-cyanobenzyl bromide by referring to the synthetic method for compound I-1 in Example 59. MS (ESI) m/z: 421.3 [M+H]⁺.

### Example 149

### 6-(4-Methylbenzyl)-3-(3-chlorobenzyl)-2,3,4,6-tetrahydropyrido[3,4-c][1,8]naphthyridine-5-(1H)-one (I-92)

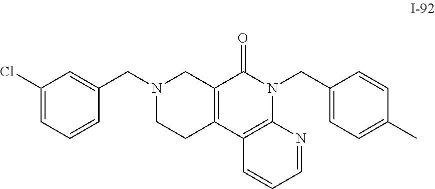 [SMILES: Cc1ccc(Cn2c(=O)c3c(c4cccnc42)CCN(Cc2cccc(Cl)c2)C3)cc1]

The Boc protecting group in compound 6-A-21 was first removed by referring to the method in Example 58, and then the compound was prepared by reacting the resulting amine with 3-chlorobenzyl bromide by referring to the synthetic method for compound I-1 in Example 59. MS (ESI) m/z: 430.3 [M+H]⁺.

### Example 150

### 6-(4-Methylbenzyl)-3-(3-fluorobenzyl)-2,3,4,6-tetrahydropyrido[3,4-c][1,8]naphthyridine-5-(1H)-one (I-93)

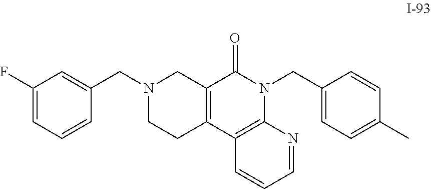 [SMILES: Cc1ccc(Cn2c(=O)c3c(c4cccnc42)CCN(Cc2cccc(F)c2)C3)cc1]

The Boc protecting group in compound 6-A-21 was first removed by referring to the method in Example 58, and then the compound was prepared by reacting the resulting amine with 3-fluorobenzyl bromide by referring to the synthetic method for compound I-1 in Example 59. MS (ESI) m/z: 414.3 [M+H]⁺.

### Example 151

### 6-(4-Ethylbenzyl)-3-(3-cyanobenzyl)-2,3,4,6-tetrahydropyrido[3,4-c][1,8]naphthyridine-5-(1H)-one (I-94)

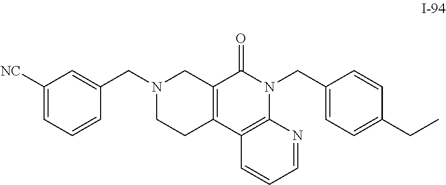 [SMILES: CCc1ccc(Cn2c(=O)c3c(c4cccnc42)CCN(Cc2cccc(C#N)c2)C3)cc1]

The Boc protecting group in compound 6-A-22 was first removed by referring to the method in Example 58, and then the compound was prepared by reacting the resulting amine with 3-cyanobenzyl bromide by referring to the synthetic method for compound I-1 in Example 59. MS (ESI) m/z: 435.3 [M+H]⁺.

### Example 152

### 6-(4-Ethylbenzyl)-3-(3-fluorobenzyl)-2,3,4,6-tetrahydropyrido[3,4-c][1,8]naphthyridine-5-(1H)-one (I-95)

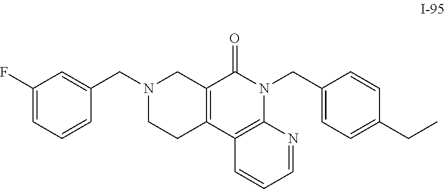 [SMILES: CCc1ccc(Cn2c(=O)c3c(c4cccnc42)CCN(Cc2cccc(F)c2)C3)cc1]

The Boc protecting group in compound 6-A-22 was first removed by referring to the method in Example 58, and then the compound was prepared by reacting the resulting amine with 3-fluorobenzyl bromide by referring to the synthetic method for compound I-1 in Example 59. MS (ESI) m/z: 428.3 [M+H]⁺.

### Example 153

### 6-(4-Ethylbenzyl)-3-(3-chlorobenzyl)-2,3,4,6-tetrahydropyrido[3,4-c][1,8]naphthyridine-5-(1H)-one (I-96)

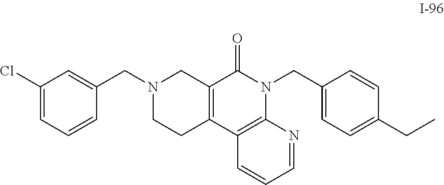 [SMILES: CCc1ccc(Cn2c(=O)c3c(c4cccnc42)CCN(Cc2cccc(Cl)c2)C3)cc1]

The Boc protecting group in compound 6-A-22 was first removed by referring to the method in Example 58, and then the compound was prepared by reacting the resulting amine with 3-chlorobenzyl bromide by referring to the synthetic method for compound I-1 in Example 59. MS (ESI) m/z: 444.3 [M+H]⁺.

### Example 154

### 3-(3-Chlorobenzyl)-6-((2,3-dihydro[1,4]benzodioxin-6-yl)methyl)-2,3,4,6-tetrahydropyrido[3,4-c][1,8]naphthyridine-5(H)-one (I-97)

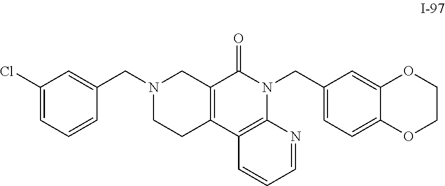 [SMILES: O=c1c2c(c3cccnc3n1Cc1ccc3c(c1)OCCO3)CCN(Cc1cccc(Cl)c1)C2]

The Boc protecting group in compound 6-A-38 was first removed by referring to the method in Example 58, and then the compound was prepared by reacting the resulting amine with 3-fluorobenzyl bromide by referring to the synthetic method for compound I-1 in Example 59. MS (ESI) m/z: 474.3 [M+H]⁺.

### Example 155

### 6-([1,3]Benzodioxol-5-ylmethyl)-3-(3-cyanobenzyl)-2,3,4,6-tetrahydropyrido[3,4-c][1,8]naphthyridine-5-(1H)-one (I-98)

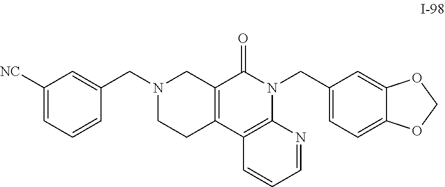 [SMILES: N#Cc1cccc(CN2CCc3c(c(=O)n(Cc4ccc5c(c4)OCO5)c4ncccc34)C2)c1]

The Boc protecting group in compound 6-A-39 was first removed by referring to the method in Example 58, and then the compound was prepared by reacting the resulting amine with 3-cyanobenzyl bromide by referring to the synthetic method for compound I-1 in Example 59. MS (ESI) m/z: 451.3 [M+H]⁺.

### Example 156

### 6-(4-Methoxybenzyl)-3-(3-fluorobenzyl)-2,3,4,6-tetrahydropyrido[3,4-c][1,8]naphthyridine-5-(1H)-one (I-99)

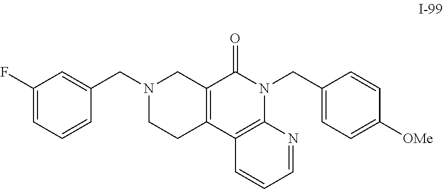 [SMILES: COc1ccc(Cn2c(=O)c3c(c4cccnc42)CCN(Cc2cccc(F)c2)C3)cc1]

The Boc protecting group in compound 6-A-23 was first removed by referring to the method in Example 58, and then the compound was prepared by reacting the resulting amine with 3-fluorobenzyl bromide by referring to the synthetic method for compound I-1 in Example 59. MS (ESI) m/z: 430.3 [M+H]⁺.

### Example 157

### 6-(4-Methoxybenzyl)-3-(3-chlorobenzyl)-2,3,4,6-tetrahydropyrido[3,4-c][1,8]naphthyridine-5-(1H)-one (I-100)

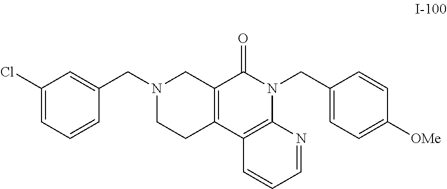 [SMILES: COc1ccc(Cn2c(=O)c3c(c4cccnc42)CCN(Cc2cccc(Cl)c2)C3)cc1]

The Boc protecting group in compound 6-A-23 was first removed by referring to the method in Example 58, and then the compound was prepared by reacting the resulting amine with 3-chlorobenzyl bromide by referring to the synthetic method for compound I-1 in Example 59. MS (ESI) m/z: 446.3 [M+H]⁺.

### 7-Benzyl-4-(4-trifluoromethylbenzyl)-6,7,8,9-tetrahydrofuro[3,4-c][2,7]naphthyridine-5(4H)-one (I-101)

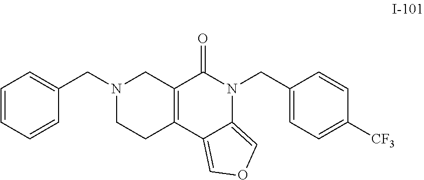 [SMILES: O=c1c2c(c3cocc3n1Cc1ccc(C(F)(F)F)cc1)CCN(Cc1ccccc1)C2]

The Boc protecting group in compound 6-D was first removed by referring to the method in Example 58, and then the compound was prepared by reacting the resulting amine with benzyl bromide by referring to the synthetic method for compound I-1 in Example 59. MS (ESI) m/z: 439.3 [M+H]⁺.

### Example 159

### 7-Benzyl-4-(4-trifluoromethylbenzyl)-6,7,8,9-tetrahydrothieno[3,4-c][2,7]naphthyridine-5(4H)-one (I-102)

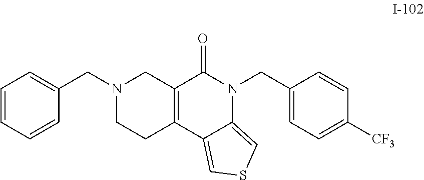 [SMILES: O=c1c2c(c3cscc3n1Cc1ccc(C(F)(F)F)cc1)CCN(Cc1ccccc1)C2]

The Boc protecting group in compound 6-E was first removed by referring to the method in Example 58, and then the compound was prepared by reacting the resulting amine with benzyl bromide by referring to the synthetic method for compound I-1 in Example 59. MS (ESI) m/z: 455.3 [M+H]⁺.

### Example 160

### 7-Benzyl-4-(4-trifluoromethylbenzyl)-6,7,8,9-tetrahydrothieno[3,2-c][2,7]naphthyridine-5(4H)-one (I-103)

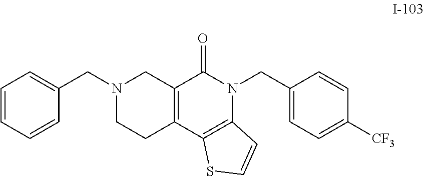 [SMILES: O=c1c2c(c3sccc3n1Cc1ccc(C(F)(F)F)cc1)CCN(Cc1ccccc1)C2]

The Boc protecting group in compound 6-F was first removed by referring to the method in Example 58, and then the compound was prepared by reacting the resulting amine with benzyl bromide by referring to the synthetic method for compound I-1 in Example 59. MS (ESI) m/z: 455.3 [M+H]⁺.

### Example 161

### 7-Benzyl-4-(4-trifluoromethylbenzyl)-6,7,8,9-tetrahydrothieno[2,3-c][2,7]naphthyridine-5(4H)-one (I-104)

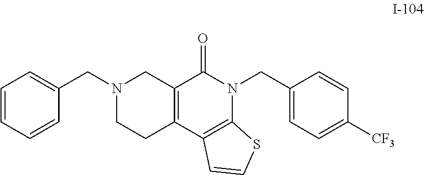 [SMILES: O=c1c2c(c3ccsc3n1Cc1ccc(C(F)(F)F)cc1)CCN(Cc1ccccc1)C2]

The Boc protecting group in compound 6-G was first removed by referring to the method in Example 58, and then the compound was prepared by reacting the resulting amine with benzyl bromide by referring to the synthetic method for compound I-1 in Example 59. MS (ESI) m/z: 455.3 [M+H]⁺.

### Example 162

### 7-Benzyl-4-(4-trifluoromethylbenzyl)-6,7,8,9-tetrahydrothiazolo[4,5-c][2,7]naphthyridine-5(4H)-one (I-105)

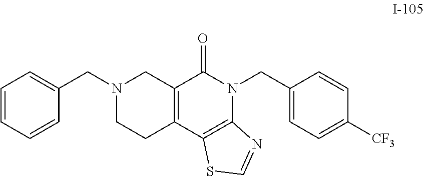 [SMILES: O=c1c2c(c3scnc3n1Cc1ccc(C(F)(F)F)cc1)CCN(Cc1ccccc1)C2]

The Boc protecting group in compound 6-H was first removed by referring to the method in Example 58, and then the compound was prepared by reacting the resulting amine with benzyl bromide by referring to the synthetic method for compound I-1 in Example 59. MS (ESI) m/z: 456.3 [M+H]⁺.

### Example 163

### Synthesis of 4-(2-methylbenzyl)-6,7,8,9-tetrahydroimidazo[1,2-a]pyrido[3,4-e]pyrimidine-5(4H)-one (12-1)

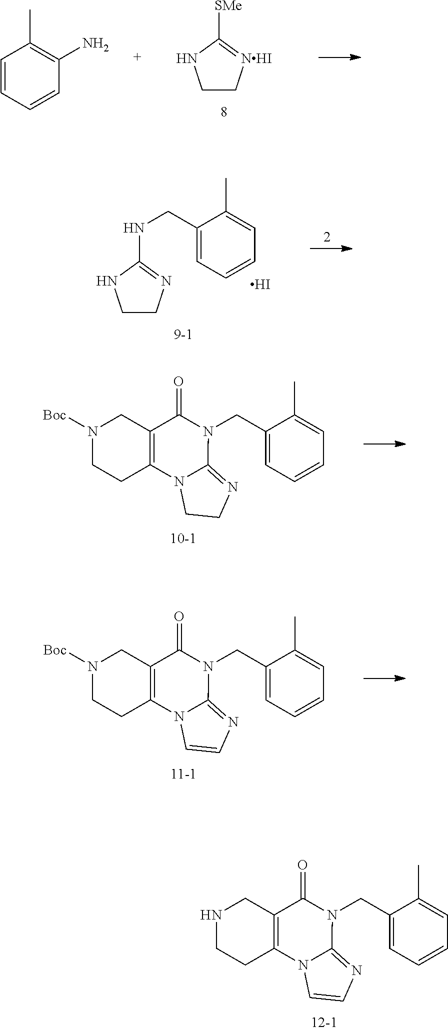

0.22 g (2 mmol) of 2-methylbenzylamine was dissolved in 10 mL of 1,4-dioxane, and 0.49 g of 2-methylthio-imidazoline hydroiodide (compound 8, 2 mmol) was added to the above solution. The mixture was heated to 70° C. and reacted for 2 h under argon atmosphere. The reaction mixture was concentrated by rotary evaporation to dryness to remove the solvent to give compound 9-1, which was directly used in the next step without purification.

The compound 9-1 obtained in the above step was dissolved in 10 mL of methanol, and 0.54 g of ethyl 1-N-tert-butoxycarbonyl-4-piperidone-3-carboxylate (compound 2, 2 mmol) and 0.16 g of sodium methoxide (3 mmol) were added to the above solution. The reaction solution was heated at reflux for 2 h. After the reaction was completed as detected by TLC, the reaction mixture was concentrated by rotary evaporation to remove the solvent, and 10 mL of water was added to the residue. The mixture was adjusted to pH 7 with 1 N hydrochloric acid and extracted with ethyl acetate (15 mL×3). The organic phases were combined, dried over anhydrous sodium sulfate, and concentrated, and the residue was purified by column chromatography to give compound 10-1 (0.44 g, yield over two steps: 56%), MS (ESI) m/z: 397.2 [M+H]⁺.

0.4 g (1 mmol) of compound 10-1 was dissolved in 3 mL of DMSO, and 0.84 g of 2-iodoxybenzoic acid (IBX, 3 mmol) was added to the above solution. The mixture was reacted at room temperature for 6 h. After the reaction was completed as detected by TLC, 20 mL of water was added to the reaction solution, and the mixture was extracted with ethyl acetate (20 mL×3). The organic phases were combined, dried over anhydrous sodium sulfate, and concentrated, and the residue was purified by column chromatography to give compound 11-1 (0.23 g, yield: 58%), MS (ESI) m/z: 395.2 [M+H]⁺, HRMS (ESI) m/z: [M+H]⁺ 395.2004, calcd. 395.2005.

0.2 g of compound 11-1 was dissolved in 3 mL of methanol, and 1 mL of a saturated solution of HCl in methanol was added. The mixture was stirred at room temperature for 12 h. The reaction mixture was concentrated by rotary evaporation to dryness to remove the solvent. 10 mL of water was added to the residue, and the mixture was neutralized with a saturated NaHCO₃ solution to pH 8 and extracted with dichloromethane (15 mL×3). The organic phases were combined, dried over anhydrous sodium sulfate, and concentrated to give compound 12-1 (0.15 g, yield: 91%), MS (ESI) m/z: 295.2 [M+H]⁺.

### Example 164

### Synthesis of 4-benzyl-6,7,8,9-tetrahydroimidazo[1,2-a]pyrido[3,4-e]pyrimidine-5(4H)-one (12-2)

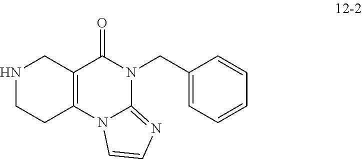 [SMILES: O=c1c2c(n3ccnc3n1Cc1ccccc1)CCNC2]

The compound was prepared by referring to the synthetic method for compound 12-1 in Example 163 with benzylamine as a starting material, MS (ESI) m/z: 281.1 [M+H]⁺.

### Synthesis of 4-(4-fluorobenzyl)-6,7,8,9-tetrahydroimidazo[1,2-a]pyrido[3,4-e]pyrimidine-5(4H)-one (12-3)

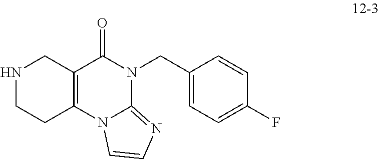 [SMILES: O=c1c2c(n3ccnc3n1Cc1ccc(F)cc1)CCNC2]

The compound was prepared by referring to the synthetic method for compound 12-1 in Example 163 with 4-fluorobenzylamine as a starting material, MS (ESI) m/z: 299.1 [M+H]⁺.

### Example 166

### Synthesis of 4-(2,4-difluorobenzyl)-6,7,8,9-tetrahydroimidazo[1,2-a]pyrido[3,4-e]pyrimidine-5(4H)-one (12-4)

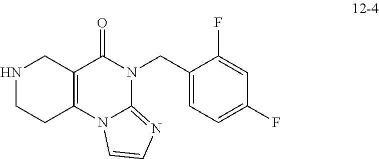 [SMILES: O=c1c2c(n3ccnc3n1Cc1ccc(F)cc1F)CCNC2]

The compound was prepared by referring to the synthetic method for compound 12-1 in Example 163 with 2,4-difluorobenzylamine as a starting material, MS (ESI) m/z: 317.1 [M+H]⁺.

### Example 167

### Synthesis of 4-(4-chlorobenzyl)-6,7,8,9-tetrahydroimidazo[1,2-a]pyrido[3,4-e]pyrimidine-5(4H)-one (12-5)

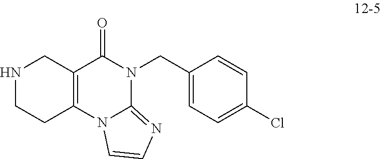 [SMILES: O=c1c2c(n3ccnc3n1Cc1ccc(Cl)cc1)CCNC2]

The compound was prepared by referring to the synthetic method for compound 12-1 in Example 163 with 4-chlorobenzylamine as a starting material, MS (ESI) m/z: 315.1 [M+H]⁺.

### Example 168

### Synthesis of 4-(4-methylbenzyl)-6,7,8,9-tetrahydroimidazo[1,2-a]pyrido[3,4-e]pyrimidine-5(4H)-one (12-6)

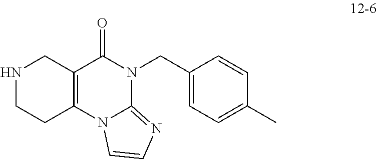 [SMILES: Cc1ccc(Cn2c(=O)c3c(n4ccnc24)CCNC3)cc1]

The compound was prepared by referring to the synthetic method for compound 12-1 in Example 163 with 4-methylbenzylamine as a starting material, MS (ESI) m/z: 295.3 [M+H]⁺.

### Example 169

### Synthesis of 4-(4-ethylbenzyl)-6,7,8,9-tetrahydroimidazo[1,2-a]pyrido[3,4-e]pyrimidine-5(4H)-one (12-7)

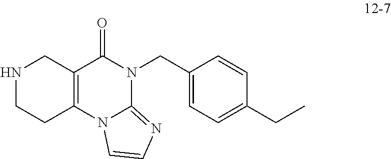 [SMILES: CCc1ccc(Cn2c(=O)c3c(n4ccnc24)CCNC3)cc1]

The compound was prepared by referring to the synthetic method for compound 12-1 in Example 163 with 4-ethylbenzylamine as a starting material, MS (ESI) m/z: 309.3 [M+H]⁺.

### Example 170

### Synthesis of 4-(4-trifluoromethylbenzyl)-6,7,8,9-tetrahydroimidazo[1,2-a]pyrido[3,4-e]pyrimidine-5(4H)-one (12-8)

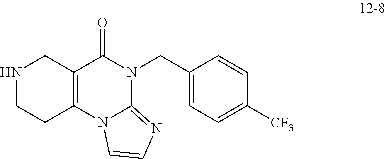 [SMILES: O=c1c2c(n3ccnc3n1Cc1ccc(C(F)(F)F)cc1)CCNC2]

The compound was prepared by referring to the synthetic method for compound 12-1 in Example 163 with 4-trifluoromethylbenzylamine as a starting material, MS (ESI) m/z: 349.1 [M+H]⁺.

### Example 171

### Synthesis of 4-(4-methoxybenzyl)-6,7,8,9-tetrahydroimidazo[1,2-a]pyrido[3,4-e]pyrimidine-5(4H)-one (12-9)

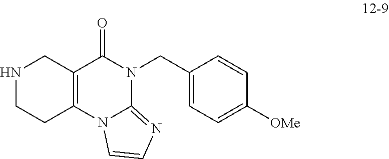 [SMILES: COc1ccc(Cn2c(=O)c3c(n4ccnc24)CCNC3)cc1]

The compound was prepared by referring to the synthetic method for compound 12-1 in Example 163 with 4-methoxybenzylamine as a starting material, MS (ESI) m/z: 311.3 [M+H]⁺.

### Example 172

### Preparation of 7-benzyl-4-(2-methylbenzyl)-6,7,8,9-tetrahydroimidazo[1,2-a]pyrido[3,4-e]pyrimidine-5(4H)-one (I-106)

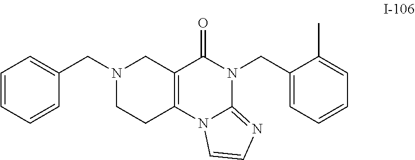 [SMILES: Cc1ccccc1Cn1c(=O)c2c(n3ccnc13)CCN(Cc1ccccc1)C2]

100 mg (0.34 mmol) of compound 12-1 was dissolved in 3 mL of DMF, and 60 mg of benzyl bromide (0.35 mmol) and 70 mg of potassium carbonate (0.5 mmol) were separately added to the above solution. The mixture was reacted at room temperature for 6 h. After the reaction was completed as detected by TLC, 15 mL of water was added to the reaction solution, and the mixture was extracted with ethyl acetate (10 mL×3). The organic phases were combined, dried over anhydrous sodium sulfate, filtered, and concentrated. The crude product was first separated by column chromatography and then further purified by HPLC (eluent: acetonitrile containing 0.1% TFA and water) to give a trifluoroacetate salt of the target compound I-82 (88 mg, yield: 52%). MS (ESI) m/z: 385.2 [M+H]⁺.

### Example 173

### Preparation of 4,7-dibenzyl-6,7,8,9-tetrahydroimidazo[1,2-a]pyrido[3,4-e]pyrimidine-5(4H)-one (I-107)

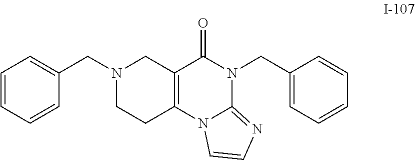 [SMILES: O=c1c2c(n3ccnc3n1Cc1ccccc1)CCN(Cc1ccccc1)C2]

The compound was prepared by reacting compound 12-2 with benzyl bromide by referring to the synthetic method for compound I-106 in Example 172, MS (ESI) m/z: 371.2 [M+H]⁺.

### Example 174

### Preparation of 7-(3-fluorobenzyl)-4-benzyl-6,7,8,9-tetrahydroimidazo[1,2-a]pyrido[3,4-e]pyrimidine-5(4H)-one (I-108)

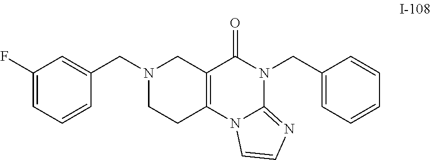 [SMILES: O=c1c2c(n3ccnc3n1Cc1ccccc1)CCN(Cc1cccc(F)c1)C2]

The compound was prepared by reacting compound 12-2 with 3-fluorobenzyl bromide by referring to the synthetic method for compound I-106 in Example 172, MS (ESI) m/z: 389.2 [M+H]⁺.

### Example 175

### Preparation of 7-(3-chlorobenzyl)-4-benzyl-6,7,8,9-tetrahydroimidazo[1,2-a]pyrido[3,4-e]pyrimidine-5(4H)-one (I-109)

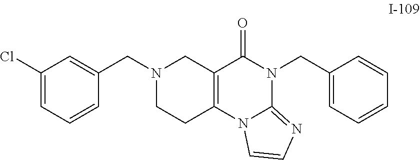 [SMILES: O=c1c2c(n3ccnc3n1Cc1ccccc1)CCN(Cc1cccc(Cl)c1)C2]

The compound was prepared by reacting compound 12-2 with 3-chlorobenzyl bromide by referring to the synthetic method for compound I-106 in Example 172, MS (ESI) m/z: 405.1 [M+H]⁺.

### Example 176

### Preparation of 7-(3-methylbenzyl)-4-benzyl-6,7,8,9-tetrahydroimidazo[1,2-a]pyrido[3,4-e]pyrimidine-5(4H)-one (I-110)

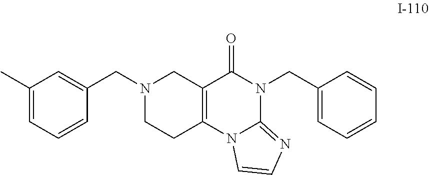 [SMILES: Cc1cccc(CN2CCc3c(c(=O)n(Cc4ccccc4)c4nccn34)C2)c1]

The compound was prepared by reacting compound 12-2 with 3-methylbenzyl bromide by referring to the synthetic method for compound I-106 in Example 172, MS (ESI) m/z: 385.2 [M+H]⁺.

### Example 177

### Preparation of 7-(3-cyanobenzyl)-4-benzyl-6,7,8,9-tetrahydroimidazo[1,2-a]pyrido[3,4-e]pyrimidine-5(4H)-one (I-111)

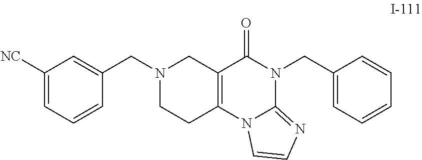 [SMILES: N#Cc1cccc(CN2CCc3c(c(=O)n(Cc4ccccc4)c4nccn34)C2)c1]

The compound was prepared by reacting compound 12-2 with 3-cyanobenzyl bromide by referring to the synthetic method for compound I-106 in Example 172, MS (ESI) m/z: 396.2 [M+H]⁺.

### Example 178

### Preparation of 7-benzyl-4-(4-fluorobenzyl)-6,7,8,9-tetrahydroimidazo[1,2-a]pyrido[3,4-e]pyrimidine-5(4H)-one (I-112)

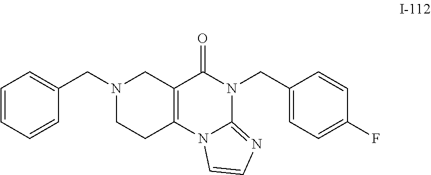 [SMILES: O=c1c2c(n3ccnc3n1Cc1ccc(F)cc1)CCN(Cc1ccccc1)C2]

The compound was prepared by reacting compound 12-3 with benzyl bromide by referring to the synthetic method for compound I-106 in Example 172, MS (ESI) m/z: 389.2 [M+H]⁺.

### Example 179

### Preparation of 7-(3-fluorobenzyl)-4-(4-fluorobenzyl)-6,7,8,9-tetrahydroimidazo[1,2-a]pyrido[3,4-e]pyrimidine-5(4H)-one (I-113)

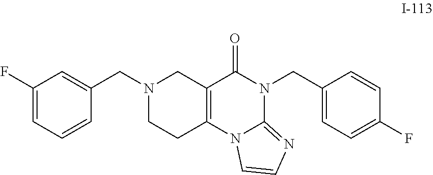 [SMILES: O=c1c2c(n3ccnc3n1Cc1ccc(F)cc1)CCN(Cc1cccc(F)c1)C2]

The compound was prepared by reacting compound 12-3 with 3-fluorobenzyl bromide by referring to the synthetic method for compound I-106 in Example 172, MS (ESI) m/z: 407.2 [M+H]⁺.

### Example 180

### Preparation of 7-(3-chlorobenzyl)-4-(4-fluorobenzyl)-6,7,8,9-tetrahydroimidazo[1,2-a]pyrido[3,4-e]pyrimidine-5(4H)-one (I-114)

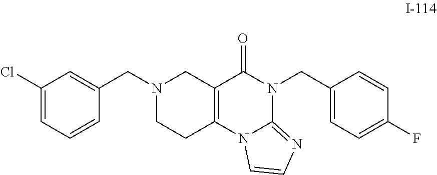 [SMILES: O=c1c2c(n3ccnc3n1Cc1ccc(F)cc1)CCN(Cc1cccc(Cl)c1)C2]

The compound was prepared by reacting compound 12-3 with 3-chlorobenzyl bromide by referring to the synthetic method for compound I-106 in Example 172, MS (ESI) m/z: 423.1 [M+H]⁺.

### Example 181

### Preparation of 7-(3-methylbenzyl)-4-(4-fluorobenzyl)-6,7,8,9-tetrahydroimidazo[1,2-a]pyrido[3,4-e]pyrimidine-5(4H)-one (I-115)

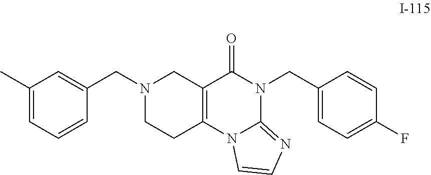 [SMILES: Cc1cccc(CN2CCc3c(c(=O)n(Cc4ccc(F)cc4)c4nccn34)C2)c1]

The compound was prepared by reacting compound 12-3 with 3-methylbenzyl bromide by referring to the synthetic method for compound I-106 in Example 172, MS (ESI) m/z: 403.2 [M+H]⁺.

### Example 182

### Preparation of 7-(3-cyanobenzyl)-4-(4-fluorobenzyl)-6,7,8,9-tetrahydroimidazo[1,2-a]pyrido[3,4-e]pyrimidine-5(4H)-one (I-116)

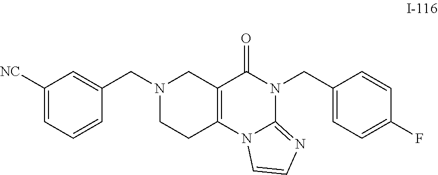 [SMILES: N#Cc1cccc(CN2CCc3c(c(=O)n(Cc4ccc(F)cc4)c4nccn34)C2)c1]

The compound was prepared by reacting compound 12-3 with 3-cyanobenzyl bromide by referring to the synthetic method for compound I-106 in Example 172, MS (ESI) m/z: 414.2 [M+H]⁺.

### Example 183

### Preparation of 7-benzyl-4-(4-chlorobenzyl)-6,7,8,9-tetrahydroimidazo[1,2-a]pyrido[3,4-e]pyrimidine-5(4H)-one (I-117)

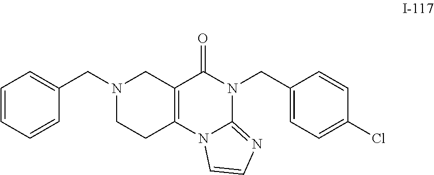 [SMILES: O=c1c2c(n3ccnc3n1Cc1ccc(Cl)cc1)CCN(Cc1ccccc1)C2]

The compound was prepared by reacting compound 12-5 with benzyl bromide by referring to the synthetic method for compound I-106 in Example 172, MS (ESI) m/z: 405.2 [M+H]⁺.

### Example 184

### Preparation of 7-(3-chlorobenzyl)-4-(4-fluorobenzyl)-6,7,8,9-tetrahydroimidazo[1,2-a]pyrido[3,4-e]pyrimidine-5(4H)-one (I-118)

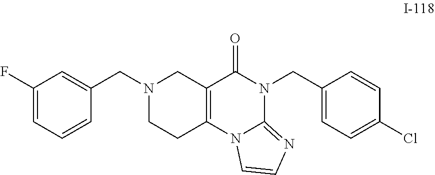 [SMILES: O=c1c2c(n3ccnc3n1Cc1ccc(Cl)cc1)CCN(Cc1cccc(F)c1)C2]

The compound was prepared by reacting compound 12-5 with 3-fluorobenzyl bromide by referring to the synthetic method for compound I-106 in Example 172, MS (ESI) m/z: 423.2 [M+H]⁺.

### Example 185

### Preparation of 7-(3-chlorobenzyl)-4-(4-chlorobenzyl)-6,7,8,9-tetrahydroimidazo[1,2-a]pyrido[3,4-e]pyrimidine-5(4H)-one (I-119)

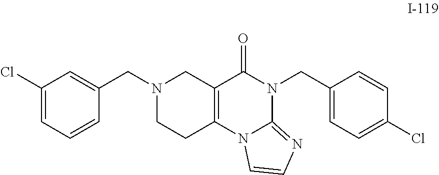 [SMILES: O=c1c2c(n3ccnc3n1Cc1ccc(Cl)cc1)CCN(Cc1cccc(Cl)c1)C2]

The compound was prepared by reacting compound 12-5 with 3-chlorobenzyl bromide by referring to the synthetic method for compound I-106 in Example 172, MS (ESI) m/z: 439.1 [M+H]⁺.

### Example 186

### Preparation of 7-(3-methylbenzyl)-4-(4-chlorobenzyl)-6,7,8,9-tetrahydroimidazo[1,2-a]pyrido[3,4-e]pyrimidine-5(4H)-one (I-120)

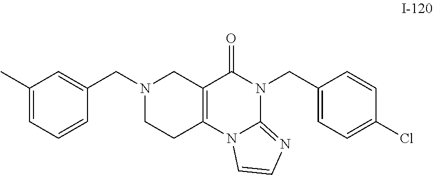 [SMILES: Cc1cccc(CN2CCc3c(c(=O)n(Cc4ccc(Cl)cc4)c4nccn34)C2)c1]

The compound was prepared by reacting compound 12-5 with 3-methylbenzyl bromide by referring to the synthetic method for compound I-106 in Example 172, MS (ESI) m/z: 419.2 [M+H]⁺.

### Example 187

### Preparation of 7-(3-cyanobenzyl)-4-(4-chlorobenzyl)-6,7,8,9-tetrahydroimidazo[1,2-a]pyrido[3,4-e]pyrimidine-5(4H)-one (I-121)

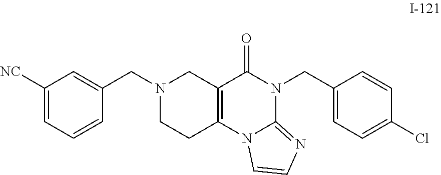 [SMILES: N#Cc1cccc(CN2CCc3c(c(=O)n(Cc4ccc(Cl)cc4)c4nccn34)C2)c1]

The compound was prepared by reacting compound 12-5 with 3-cyanobenzyl bromide by referring to the synthetic method for compound I-106 in Example 172, MS (ESI) m/z: 430.2 [M+H]⁺.

### Example 188

### Preparation of 7-benzyl-4-(2,4-difluorobenzyl)-6,7,8,9-tetrahydroimidazo[1,2-a]pyrido[3,4-e]pyrimidine-5(4H)-one (I-122)

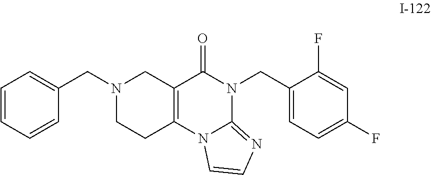 [SMILES: O=c1c2c(n3ccnc3n1Cc1ccc(F)cc1F)CCN(Cc1ccccc1)C2]

The compound was prepared by reacting compound 12-4 with benzyl bromide by referring to the synthetic method for compound I-106 in Example 172, MS (ESI) m/z: 407.2 [M+H]⁺.

### Example 189

### Preparation of 7-(3-fluorobenzyl)-4-(2,4-difluorobenzyl)-6,7,8,9-tetrahydroimidazo[1,2-a]pyrido[3,4-e]pyrimidine-5(4H)-one (I-123)

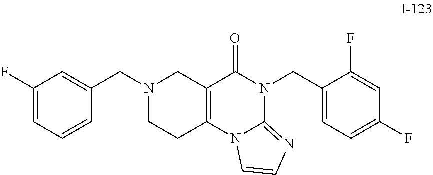 [SMILES: O=c1c2c(n3ccnc3n1Cc1ccc(F)cc1F)CCN(Cc1cccc(F)c1)C2]

The compound was prepared by reacting compound 12-4 with 3-fluorobenzyl bromide by referring to the synthetic method for compound I-106 in Example 172, MS (ESI) m/z: 425.2 [M+H]⁺.

### Example 190

### Preparation of 7-(3-chlorobenzyl)-4-(2,4-difluorobenzyl)-6,7,8,9-tetrahydroimidazo[1,2-a]pyrido[3,4-e]pyrimidine-5(4H)-one (I-124)

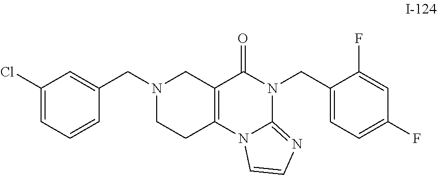 [SMILES: O=c1c2c(n3ccnc3n1Cc1ccc(F)cc1F)CCN(Cc1cccc(Cl)c1)C2]

The compound was prepared by reacting compound 12-4 with 3-chlorobenzyl bromide by referring to the synthetic method for compound I-106 in Example 172, MS (ESI) m/z: 441.1 [M+H]⁺.

### Example 191

### Preparation of 7-(3-methylbenzyl)-4-(2,4-difluorobenzyl)-6,7,8,9-tetrahydroimidazo[1,2-a]pyrido[3,4-e]pyrimidine-5(4H)-one (I-125)

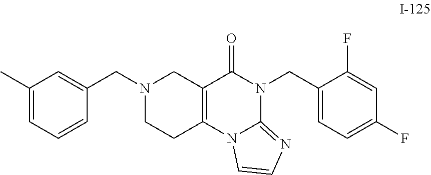 [SMILES: Cc1cccc(CN2CCc3c(c(=O)n(Cc4ccc(F)cc4F)c4nccn34)C2)c1]

The compound was prepared by reacting compound 12-4 with 3-methylbenzyl bromide by referring to the synthetic method for compound I-106 in Example 172, MS (ESI) m/z: 421.2 [M+H]⁺.

### Example 192

### Preparation of 7-(3-cyanobenzyl)-4-(2,4-difluorobenzyl)-6,7,8,9-tetrahydroimidazo[1,2-a]pyrido[3,4-e]pyrimidine-5(4H)-one (I-126)

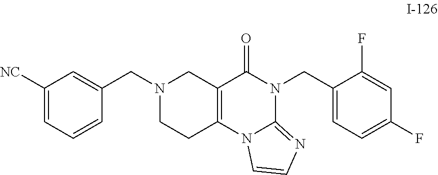 [SMILES: N#Cc1cccc(CN2CCc3c(c(=O)n(Cc4ccc(F)cc4F)c4nccn34)C2)c1]

Compound I-126 was prepared by reacting compound 12-4 with 3-cyanobenzyl bromide by the same method as that for compound I-106 in Example 172, MS (ESI) m/z: 432.2 [M+H]⁺.

### Example 193

### Preparation of 7-benzyl-4-(4-trifluoromethylbenzyl)-6,7,8,9-tetrahydroimidazo[1,2-a]pyrido[3,4-e]pyrimidine-5(4H)-one (I-127)

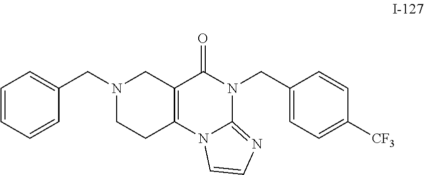 [SMILES: O=c1c2c(n3ccnc3n1Cc1ccc(C(F)(F)F)cc1)CCN(Cc1ccccc1)C2]

The compound was prepared by reacting compound 12-8 with benzyl bromide by referring to the synthetic method for compound I-106 in Example 172, MS (ESI) m/z: 439.2 [M+H]⁺.

### Example 194

### Preparation of 7-(3-fluorobenzyl)-4-(4-trifluoromethylbenzyl)-6,7,8,9-tetrahydroimidazo[1,2-a]pyrido[3,4-e]pyrimidine-5(4H)-one (I-128)

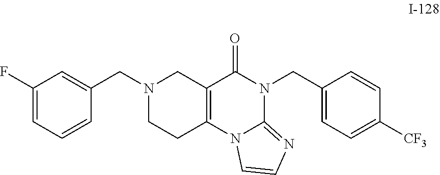 [SMILES: O=c1c2c(n3ccnc3n1Cc1ccc(C(F)(F)F)cc1)CCN(Cc1cccc(F)c1)C2]

The compound was prepared by reacting compound 12-8 with 3-fluorobenzyl bromide by referring to the synthetic method for compound I-106 in Example 172, MS (ESI) m/z: 457.2 [M+H]⁺.

### Example 195

### Preparation of 7-(3-chlorobenzyl)-4-(4-trifluoromethylbenzyl)-6,7,8,9-tetrahydroimidazo[1,2-a]pyrido[3,4-e]pyrimidine-5(4H)-one (I-129)

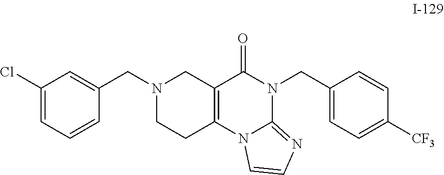 [SMILES: O=c1c2c(n3ccnc3n1Cc1ccc(C(F)(F)F)cc1)CCN(Cc1cccc(Cl)c1)C2]

The compound was prepared by reacting compound 12-8 with 3-chlorobenzyl bromide by referring to the synthetic method for compound I-106 in Example 172, MS (ESI) m/z: 473.2 [M+H]⁺.

### Example 196

### Preparation of 7-(3-methylbenzyl)-4-(4-trifluoromethylbenzyl)-6,7,8,9-tetrahydroimidazo[1,2-a]pyrido[3,4-e]pyrimidine-5(4H)-one (I-130)

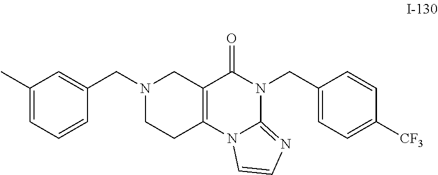 [SMILES: Cc1cccc(CN2CCc3c(c(=O)n(Cc4ccc(C(F)(F)F)cc4)c4nccn34)C2)c1]

The compound was prepared by reacting compound 12-8 with 3-methylbenzyl bromide by referring to the synthetic method for compound I-106 in Example 172, MS (ESI) m/z: 453.2 [M+H]⁺.

### Example 197

### Preparation of 7-(3-cyanobenzyl)-4-(4-trifluoromethylbenzyl)-6,7,8,9-tetrahydroimidazo[1,2-a]pyrido[3,4-e]pyrimidine-5(4H)-one (I-131)

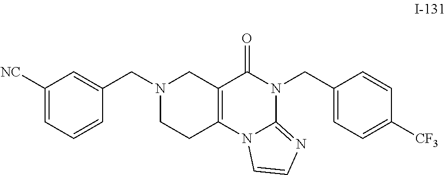 [SMILES: N#Cc1cccc(CN2CCc3c(c(=O)n(Cc4ccc(C(F)(F)F)cc4)c4nccn34)C2)c1]

The compound was prepared by reacting compound 12-8 with 3-cyanobenzyl bromide by referring to the synthetic method for compound I-106 in Example 172, MS (ESI) m/z: 464.2 [M+H]⁺.

### Example 198

### Preparation of 7-benzyl-4-(4-methylbenzyl)-6,7,8,9-tetrahydroimidazo[1,2-a]pyrido[3,4-e]pyrimidine-5(4H)-one (I-132)

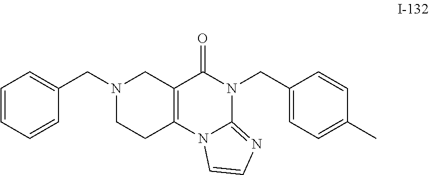 [SMILES: Cc1ccc(Cn2c(=O)c3c(n4ccnc24)CCN(Cc2ccccc2)C3)cc1]

The compound was prepared by reacting compound 12-6 with benzyl bromide by referring to the synthetic method for compound I-106 in Example 172, MS (ESI) m/z: 385.2 [M+H]⁺.

### Example 199

### Preparation of 7-(3-fluorobenzyl)-4-(4-methylbenzyl)-6,7,8,9-tetrahydroimidazo[1,2-a]pyrido[3,4-e]pyrimidine-5(4H)-one (I-133)

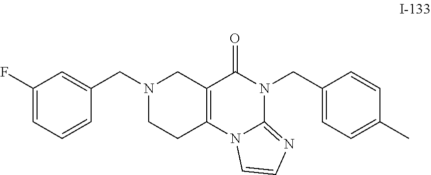 [SMILES: Cc1ccc(Cn2c(=O)c3c(n4ccnc24)CCN(Cc2cccc(F)c2)C3)cc1]

The compound was prepared by reacting compound 12-6 with 3-fluorobenzyl bromide by referring to the synthetic method for compound I-106 in Example 172, MS (ESI) m/z: 403.2 [M+H]⁺.

### Example 200

### Preparation of 7-(3-chlorobenzyl)-4-(4-methylbenzyl)-6,7,8,9-tetrahydroimidazo[1,2-a]pyrido[3,4-e]pyrimidine-5(4H)-one (I-134)

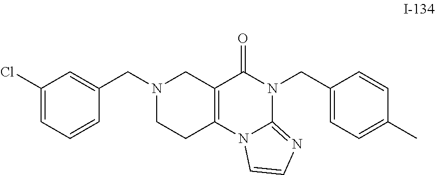 [SMILES: Cc1ccc(Cn2c(=O)c3c(n4ccnc24)CCN(Cc2cccc(Cl)c2)C3)cc1]

The compound was prepared by reacting compound 12-6 with 3-chlorobenzyl bromide by referring to the synthetic method for compound I-106 in Example 172, MS (ESI) m/z: 419.2 [M+H]⁺.

### Example 201

### Preparation of 7-(3-methylbenzyl)-4-(4-methylbenzyl)-6,7,8,9-tetrahydroimidazo[1,2-a]pyrido[3,4-e]pyrimidine-5(4H)-one (I-135)

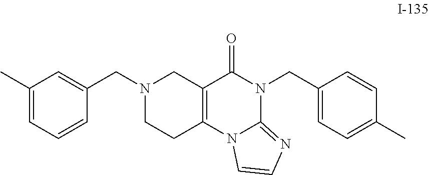

The compound was prepared by reacting compound 12-6 with 3-methylbenzyl bromide by referring to the synthetic method for compound I-106 in Example 172, MS (ESI) m/z: 399.2 [M+H]⁺.

### Example 202

### Preparation of 7-(3-cyanobenzyl)-4-(4-methylbenzyl)-6,7,8,9-tetrahydroimidazo[1,2-a]pyrido[3,4-e]pyrimidine-5(4H)-one (I-136)

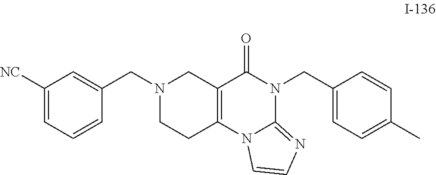 [SMILES: Cc1ccc(Cn2c(=O)c3c(n4ccnc24)CCN(Cc2cccc(C#N)c2)C3)cc1]

The compound was prepared by reacting compound 12-6 with 3-cyanobenzyl bromide by referring to the synthetic method for compound I-106 in Example 172, MS (ESI) m/z: 410.2 [M+H]⁺.

### Example 203

### Preparation of 7-benzyl-4-(4-ethylbenzyl)-6,7,8,9-tetrahydroimidazo[1,2-a]pyrido[3,4-e]pyrimidine-5(4H)-one (I-137)

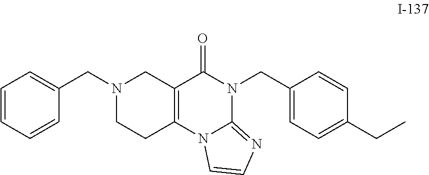 [SMILES: CCc1ccc(Cn2c(=O)c3c(n4ccnc24)CCN(Cc2ccccc2)C3)cc1]

The compound was prepared by reacting compound 12-7 with benzyl bromide by referring to the synthetic method for compound I-106 in Example 172, MS (ESI) m/z: 399.2 [M+H]⁺.

### Example 204

### Preparation of 7-(3-fluorobenzyl)-4-(4-ethylbenzyl)-6,7,8,9-tetrahydroimidazo[1,2-a]pyrido[3,4-e]pyrimidine-5(4H)-one (I-138)

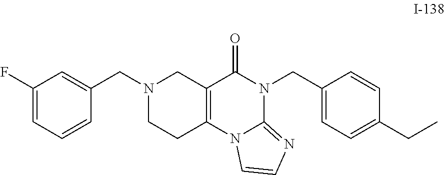 [SMILES: CCc1ccc(Cn2c(=O)c3c(n4ccnc24)CCN(Cc2cccc(F)c2)C3)cc1]

The compound was prepared by reacting compound 12-7 with 3-fluorobenzyl bromide by referring to the synthetic method for compound I-106 in Example 172, MS (ESI) m/z: 417.2 [M+H]⁺.

### Example 205

### Preparation of 7-(3-chlorobenzyl)-4-(4-ethylbenzyl)-6,7,8,9-tetrahydroimidazo[1,2-a]pyrido[3,4-e]pyrimidine-5(4H)-one (I-139)

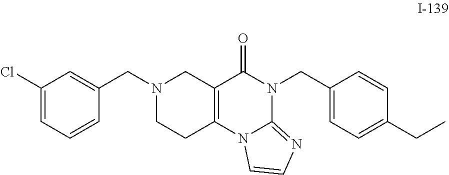 [SMILES: CCc1ccc(Cn2c(=O)c3c(n4ccnc24)CCN(Cc2cccc(Cl)c2)C3)cc1]

The compound was prepared by reacting compound 12-7 with 3-chlorobenzyl bromide by referring to the synthetic method for compound I-106 in Example 172, MS (ESI) m/z: 433.2 [M+H]⁺.

### Example 206

### Preparation of 7-(3-methylbenzyl)-4-(4-ethylbenzyl)-6,7,8,9-tetrahydroimidazo[1,2-a]pyrido[3,4-e]pyrimidine-5(4H)-one (I-140)

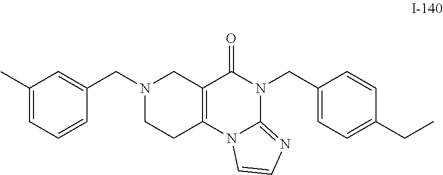 [SMILES: CCc1ccc(Cn2c(=O)c3c(n4ccnc24)CCN(Cc2cccc(C)c2)C3)cc1]

The compound was prepared by reacting compound 12-7 with 3-methylbenzyl bromide by referring to the synthetic method for compound I-106 in Example 172, MS (ESI) m/z: 413.2 [M+H]⁺.

### Example 207

### Preparation of 7-(3-cyanobenzyl)-4-(4-ethylbenzyl)-6,7,8,9-tetrahydroimidazo[1,2-a]pyrido[3,4-e]pyrimidine-5(4H)-one (I-141)

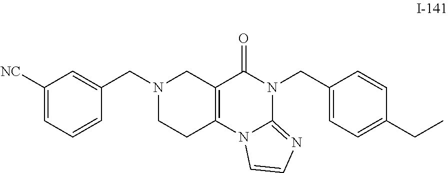 [SMILES: CCc1ccc(Cn2c(=O)c3c(n4ccnc24)CCN(Cc2cccc(C#N)c2)C3)cc1]

The compound was prepared by reacting compound 12-7 with 3-cyanobenzyl bromide by referring to the synthetic method for compound I-106 in Example 172, MS (ESI) m/z: 424.2 [M+H]⁺.

### Example 208

### Preparation of 7-benzyl-4-(4-methoxybenzyl)-6,7,8,9-tetrahydroimidazo[1,2-a]pyrido[3,4-e]pyrimidine-5(4H)-one (I-142)

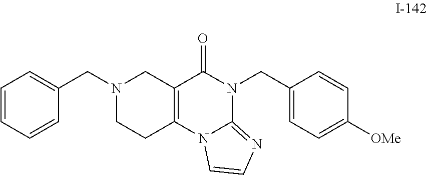 [SMILES: COc1ccc(Cn2c(=O)c3c(n4ccnc24)CCN(Cc2ccccc2)C3)cc1]

The compound was prepared by reacting compound 12-9 with benzyl bromide by referring to the synthetic method for compound I-106 in Example 172, MS (ESI) m/z: 401.2 [M+H]⁺.

### Example 209

### Preparation of 7-(3-fluorobenzyl)-4-(4-methoxybenzyl)-6,7,8,9-tetrahydroimidazo[1,2-a]pyrido[3,4-e]pyrimidine-5(4H)-one (I-143)

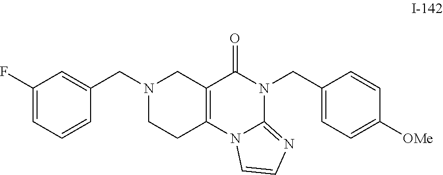 [SMILES: COc1ccc(Cn2c(=O)c3c(n4ccnc24)CCN(Cc2cccc(F)c2)C3)cc1]

The compound was prepared by reacting compound 12-9 with 3-fluorobenzyl bromide by referring to the synthetic method for compound I-106 in Example 172, MS (ESI) m/z: 419.2 [M+H]⁺.

### Example 210

### Preparation of 7-(3-chlorobenzyl)-4-(4-methoxybenzyl)-6,7,8,9-tetrahydroimidazo[1,2-a]pyrido[3,4-e]pyrimidine-5(4H)-one (I-144)

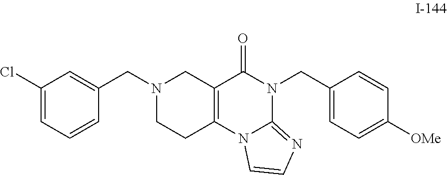 [SMILES: COc1ccc(Cn2c(=O)c3c(n4ccnc24)CCN(Cc2cccc(Cl)c2)C3)cc1]

The compound was prepared by reacting compound 12-9 with 3-chlorobenzyl bromide by referring to the synthetic method for compound I-106 in Example 172, MS (ESI) m/z: 435.2 [M+H]⁺.

### Example 210

### Preparation of 7-(3-methylbenzyl)-4-(4-methoxybenzyl)-6,7,8,9-tetrahydroimidazo[1,2-a]pyrido[3,4-e]pyrimidine-5(4H)-one (I-145)

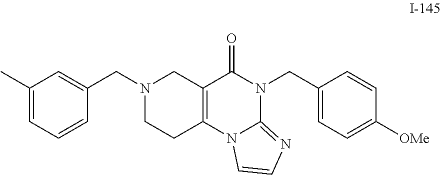 [SMILES: COc1ccc(Cn2c(=O)c3c(n4ccnc24)CCN(Cc2cccc(C)c2)C3)cc1]

The compound was prepared by reacting compound 12-9 with 3-methylbenzyl bromide by referring to the synthetic method for compound I-106 in Example 172, MS (ESI) m/z: 415.2 [M+H]⁺.

### Example 211

### Preparation of 7-(3-cyanobenzyl)-4-(4-methoxybenzyl)-6,7,8,9-tetrahydroimidazo[1,2-a]pyrido[3,4-e]pyrimidine-5(4H)-one (I-146)

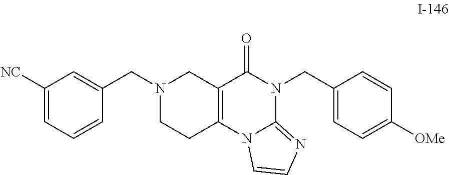 [SMILES: COc1ccc(Cn2c(=O)c3c(n4ccnc24)CCN(Cc2cccc(C#N)c2)C3)cc1]

The compound was prepared by reacting compound 12-9 with 3-cyanobenzyl bromide by referring to the synthetic method for compound I-106 in Example 172, MS (ESI) m/z: 426.2 [M+H]⁺.

### Example 212

### Synthesis of 6,7,8,9-tetrahydroimidazo[1,2-a]pyrido[3,4-e]pyrimidine-5(4H)-one (17-1)

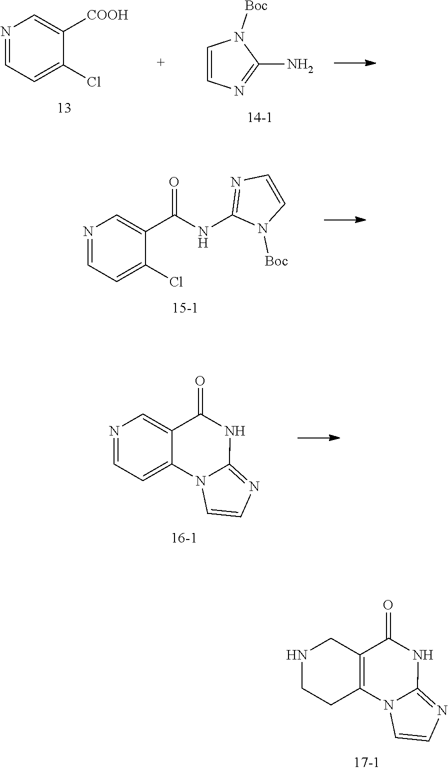 [SMILES: O=C(O)c1cnccc1Cl ; CC(C)(C)OC(=O)n1ccnc1N ; CC(C)(C)OC(=O)n1ccnc1NC(=O)c1cnccc1Cl ; O=c1[nH]c2nccn2c2ccncc12 ; O=c1[nH]c2nccn2c2c1CNCC2]

0.63 g of compound 13 (4 mmol) was dissolved in 10 mL of dichloromethane, and 2 mL of oxalyl chloride and two drops of DMF were added to the solution at 0° C. under stirring. The reaction solution was warmed to room temperature, stirred for half an hour, and concentrated by rotary evaporation under reduced pressure to remove the solvent and excessive oxalyl chloride. The resulting acyl chloride was re-dissolved in 5 mL of dichloromethane for later use. 0.73 g of compound 14-1 was dissolved in 10 mL of dichloromethane, and 1.5 mL of Et₃N was added. The solution was cooled to 0° C., and an acyl chloride solution prepared from compound 13 was slowly added to the above solution under stirring. The mixture was stirred at room temperature for 3 h, and concentrated by rotary evaporation to remove the solvent, and the residue was separated by column chromatography to give compound 15-1, MS (ESI) m/z: 323.1 [M+H]⁺.

Compound 15-1 obtained in the above step was dissolved in 5 mL of dichloromethane, and 10 mL of trifluoroacetic acid was added at 0° C. under stirring. The reaction solution was warmed to room temperature, stirred for 1 h, and concentrated by rotary evaporation to remove the solvent. The residue was dissolved in 15 mL of 1,4-dioxane, and 0.4 g of NaH (purity: 60%) was added to the above solution in portions, heated to 80° C., and stirred overnight under nitrogen atmosphere. 10 mL of methanol was added to the above reaction solution to destroy excess sodium hydride, and then the mixture was concentrated by rotary evaporation to remove the solvent. The residue was purified by column chromatography to give compound 16-1, MS (ESI) m/z: 187.1 [M+H]⁺.

Compound 16-1 obtained in the above reaction was dissolved in 15 mL of methanol, and 0.5 mL of concentrated hydrochloric acid was added, followed by the addition of 50 mg of 10% Pd—C under nitrogen atmosphere. The mixture was stirred overnight under hydrogen atmosphere and filtered through celite to remove the solid. The filtrate was concentrated to give a hydrochloride salt of compound 17-1, MS (ESI) m/z: 191.1 [M+H]⁺.

### Example 213

### Synthesis of 6,7,8,9-tetrahydropyrido[3,4-e][1,2,4]triazolo[1,5-a]pyrimidine-5(4H)-one (17-2)

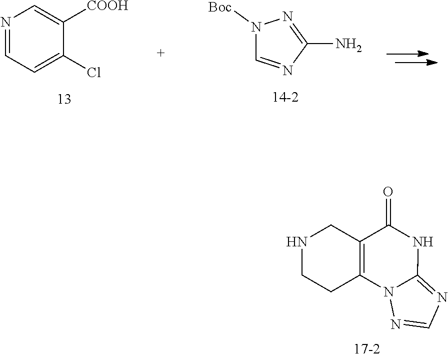 [SMILES: O=C(O)c1cnccc1Cl ; CC(C)(C)OC(=O)n1cnc(N)n1 ; O=c1[nH]c2ncnn2c2c1CNCC2]

The compound was prepared by referring to the synthetic method for compound 17-1 in Example 212 with compound 13 and compound 14-2 as starting materials, MS (ESI) m/z: 192.1 [M+H]⁺.

### Example 214

### Synthesis of 6,7,8,9-tetrahydropyrazolo[1,5-a]pyrido[3,4-e]pyrimidine-5(4H)-one (17-3)

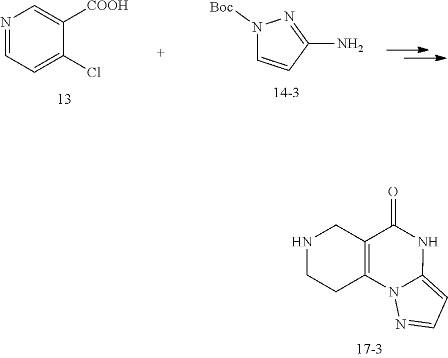 [SMILES: O=C(O)c1cnccc1Cl ; CC(C)(C)OC(=O)n1ccc(N)n1 ; O=c1[nH]c2ccnn2c2c1CNCC2]

The compound was prepared by referring to the synthetic method for compound 17-1 in Example 212 with compound 13 and compound 14-3 as starting materials, MS (ESI) m/z: 191.1 [M+H]⁺.

### Synthesis of 6,7,8,9-tetrahydroimidazo[1,5-a]pyrido[3,4-e]pyrimidine-5(4H)-one (17-4)

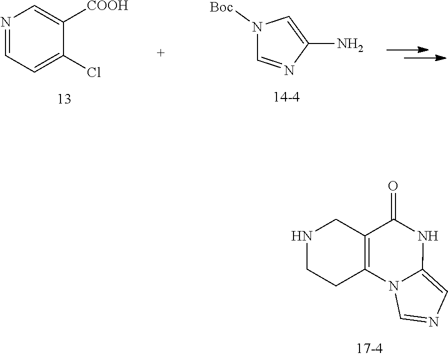 [SMILES: O=C(O)c1cnccc1Cl ; CC(C)(C)OC(=O)n1cnc(N)c1 ; O=c1[nH]c2cncn2c2c1CNCC2]

The compound was prepared by referring to the synthetic method for compound 17-1 in Example 212 with compound 13 and compound 14-4 as starting materials, MS (ESI) m/z: 191.1 [M+H]⁺.

### Example 216

### Synthesis of 1,2,3,4-tetrahydropyrido[3,4-e]pyrrolo[1,2-a]pyrimidine-5(6H)-one (17-5)

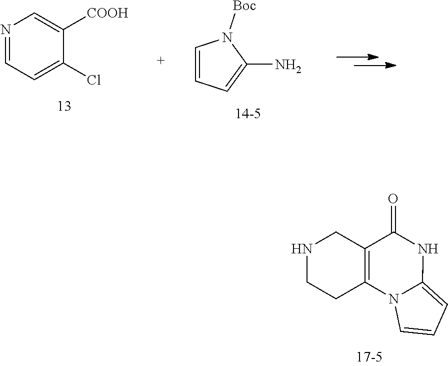 [SMILES: O=C(O)c1cnccc1Cl ; CC(C)(C)OC(=O)n1cccc1N ; O=c1[nH]c2cccn2c2c1CNCC2]

The compound was prepared by referring to the synthetic method for compound 17-1 in Example 212 with compound 13 and compound 14-5 as starting materials, MS (ESI) m/z: 190.1 [M+H]⁺.

### Example 217

### Synthesis of 4-(2,4-dimethoxybenzyl)-6,7,8,9-tetrahydroimidazo[1,2-a]pyrido[3,4-e]pyrimidine-5(4H)-one (24-1)

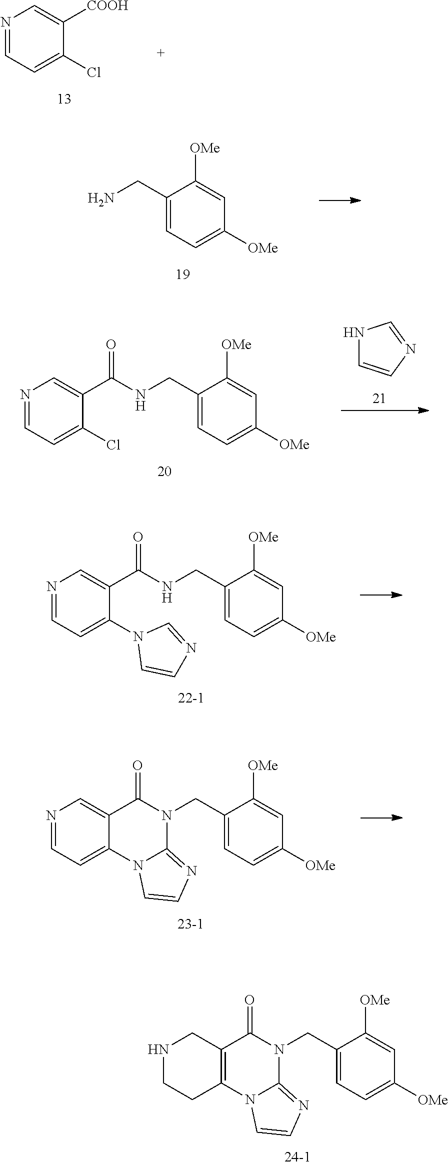

0.63 g of compound 13 (4 mmol) was dissolved in 10 mL of dichloromethane, and 2 mL of oxalyl chloride and two drops of DMF were added to the above solution under stirring at 0° C. The mixture was warmed to room temperature, stirred for half an hour, and concentrated by rotary evaporation under reduced pressure to remove the solvent and excess oxalyl chloride. The resulting acyl chloride was re-dissolved in 5 mL of dichloromethane, and the above acyl chloride solution was slowly added to 10 mL of dichloromethane solution containing 0.67 g of 2,4-dimethoxybenzylamine and 1.5 mL of triethylamine which cooled in an ice bath. The reaction solution was warmed to room temperature, stirred for 3 h, and concentrated by rotary evaporation to remove the solvent. The residue was separated by column chromatography to give compound 20, MS (ESI) m/z: 307.1 [M+H]⁺.

Compound 20 obtained in the above step was dissolved in 15 mL of DMF, and 0.5 g of imidazole and 0.4 g of NaH (purity: 60%) were separately added to the solution. The mixture was heated to 100° C., and stirred overnight under nitrogen atmosphere. After cooling to room temperature, 30 mL of water was added to the above reaction solution, and the mixture was extracted with ethyl acetate (30 mL×3). The organic phases were combined, washed once with a saturated sodium chloride solution, dried over anhydrous sodium sulfate, and concentrated by rotary evaporation to remove the solvent. The residue was purified by column chromatography to give compound 22-1, MS (ESI) m/z: 339.2 [M+H]⁺.

Compound 22-1 obtained in the above step was dissolved in 15 mL of DMF, and 0.1 g of CuI, 0.5 g of potassium tert-butoxide, and 0.2 g of 1,10-phenanthroline were separately added to the solution. The resulting mixture was reacted at 120° C. overnight under oxygen atmosphere. After the reaction was completed, the reaction mixture was cooled to room temperature. 30 mL of water was added, and the mixture was extracted with ethyl acetate (30 mL×3). The organic phases were combined, washed once with a saturated sodium chloride solution, dried over anhydrous sodium sulfate, and concentrated by rotary evaporation to remove the solvent. The residue was purified by column chromatography to give compound 23-1, MS (ESI) m/z: 337.2 [M+H]⁺.

Compound 23-1 obtained in the above reaction was dissolved in 15 mL of methanol, and 0.5 mL of concentrated hydrochloric acid was added, followed by the addition of 50 mg of 10% Pd—C under nitrogen atmosphere. The mixture was stirred overnight under hydrogen atmosphere and filtered through celite to remove the solid. The filtrate was concentrated to give a hydrochloride salt of compound 24-1, MS (ESI) m/z: 341.2 [M+H]⁺.

### Example 218

### Synthesis of 4-(2,4-dimethoxybenzyl)-6,7,8,9-tetrahydropyrido[3,4-e][1,2,4]triazolo[1,5-a]pyrimidine-5(4H)-one (24-2)

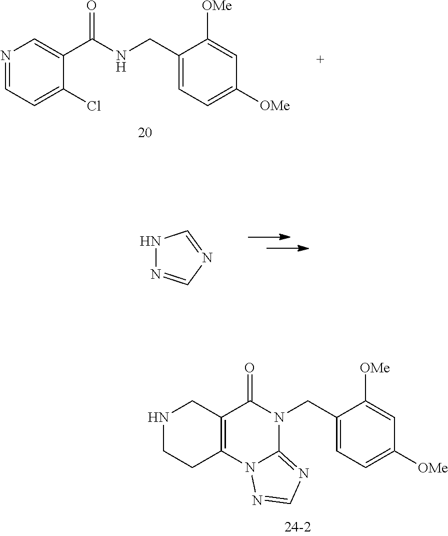 [SMILES: COc1ccc(CNC(=O)c2cnccc2Cl)c(OC)c1 ; c1nc[nH]n1 ; COc1ccc(Cn2c(=O)c3c(n4ncnc24)CCNC3)c(OC)c1]

The compound was prepared by referring to the synthetic method for compound 24-1 in Example 217 with compound 20 and 1,2,4-triazole as starting materials, MS (ESI) m/z: 342.2 [M+H]⁺.

### Example 219

### Synthesis of 4-(2,4-dimethoxybenzyl)-6,7,8,9-tetrahydropyrazolo[1,5-a]pyrido[3,4-e]pyrimidine-5(4H)-one (24-3)

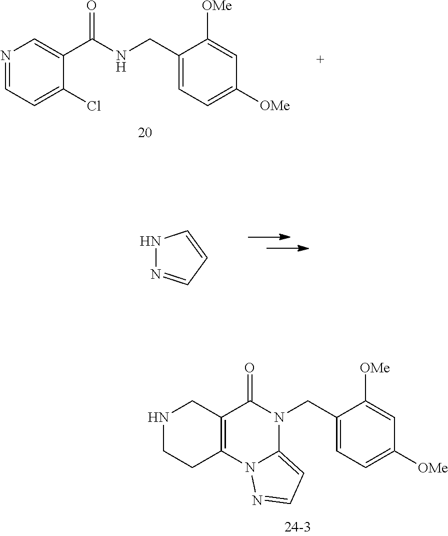 [SMILES: COc1ccc(CNC(=O)c2cnccc2Cl)c(OC)c1 ; c1cn[nH]c1 ; COc1ccc(Cn2c(=O)c3c(n4nccc24)CCNC3)c(OC)c1]

The compound was prepared by referring to the synthetic method for compound 24-1 in Example 217 with compound 20 and pyrazole as starting materials, MS (ESI) m/z: 341.2 [M+H]⁺.

### Synthesis of 7-(3-chlorobenzyl)-4-(benzo[1,3]dioxopentan-5-ylmethyl)-6,7,8,9-tetrahydroimidazo[1,2-a]pyrido[3,4-e]pyrimidine-5(4H)-one (I-147)

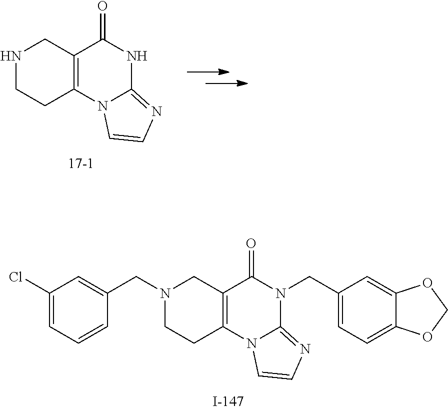 [SMILES: O=c1[nH]c2nccn2c2c1CNCC2 ; O=c1c2c(n3ccnc3n1Cc1ccc3c(c1)OCO3)CCN(Cc1cccc(Cl)c1)C2]

190 mg of compound 17-1 was dissolved in 10 mL of methanol, and 140 mg of m-chlorobenzaldehyde and 120 mg of NaBH₃CN were separately added to the solution, followed by the addition of a drop of concentrated sulfuric acid. The mixture was reacted at room temperature for 3 h. 1 mL of water was added, and the mixture was concentrated by rotary evaporation to remove the solvent. 15 mL of water was added to the residue, and the mixture was extracted with ethyl acetate (20 mL×3). The organic phases were combined, washed once with a saturated sodium chloride solution, dried over anhydrous sodium sulfate, and concentrated by rotary evaporation to remove the solvent. The residue was dissolved in 5 mL of DMF, 0.2 g of 5-bromomethylbenzo[1,3]dioxolane and 0.2 g of potassium carbonate were separately added, and the mixture was stirred at room temperature overnight. 10 mL of water was added, and the mixture was extracted with ethyl acetate (10 mL×3). The organic phases were combined, washed with saturated brine, dried over anhydrous sodium sulfate, and concentrated by rotary evaporation under reduced pressure to remove the solvent. The residue was first purified by silica gel column chromatography and then further purified by semi-preparative HPLC to give compound I-147. MS (ESI) m/z: 449.2 [M+H]⁺.

### Example 221

### Synthesis of 7-(3-chlorobenzyl)-4-((2,3-dihydro[1,4]benzodioxin-6-yl)methyl)-6,7,8,9-tetrahydroimidazo[1,2-a]pyrido[3,4-e]pyrimidine-5(4H)-one (I-148)

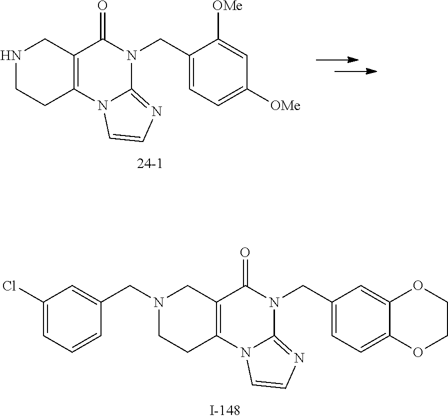 [SMILES: COc1ccc(Cn2c(=O)c3c(n4ccnc24)CCNC3)c(OC)c1 ; O=c1c2c(n3ccnc3n1Cc1ccc3c(c1)OCCO3)CCN(Cc1cccc(Cl)c1)C2]

170 mg of compound 24-1 was dissolved in 5 mL of DMF, and 120 mg of 3-chlorobenzyl bromide and 0.2 mL of triethylamine were separately added to the solution. The mixture was stirred at room temperature overnight. 5 mL of water was added, and the mixture was extracted with ethyl acetate (10 mL×3). The organic phases were combined, washed with saturated brine, dried over anhydrous sodium sulfate, and concentrated by rotary evaporation under reduced pressure to remove the solvent. The residue was purified by silica gel column chromatography, and the resulting product was dissolved in 2 mL of dichloromethane. 10 mL of trifluoroacetic acid was added to the solution, and the mixture was stirred at room temperature for 2 h and concentrated by rotary evaporation to remove the solvent and excess trifluoroacetic acid. The residue was dissolved in 5 mL of DMF, and 120 mg of 6-bromomethyl[2,3]dihydrobenzo[1,4]dioxin and 0.2 g of potassium carbonate were separately added to the solution. The mixture was stirred at room temperature overnight, 10 mL of water was added, and the mixture was extracted with ethyl acetate (10 mL×3). The organic phases were combined, washed with saturated brine, dried over anhydrous sodium sulfate, and concentrated by rotary evaporation under reduced pressure to remove the solvent. The residue was first purified by silica gel column chromatography and then further purified by semi-preparative HPLC to give compound I-148. MS (ESI) m/z: 463.2 [M+H]⁺.

### Example 222

### Preparation of 4-(4-trifluoromethylbenzyl)-7-benzyl-6,7,8,9-tetrahydropyrido[3,4-e][1,2,4]triazolo[1,5-a]pyrimidine-5(4H)-one (I-149)

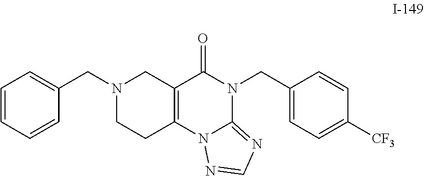 [SMILES: O=c1c2c(n3ncnc3n1Cc1ccc(C(F)(F)F)cc1)CCN(Cc1ccccc1)C2]

The compound was prepared from compound 17-2 by referring to the synthetic method for compound I-147 in Example 220, MS (ESI) m/z: 440.2 [M+H]⁺.

### Example 223

### Preparation of 4-(4-trifluoromethylbenzyl)-7-(3-chlorobenzyl)-6,7,8,9-tetrahydropyrido[3,4-e][1,2,4]triazolo[1,5-a]pyrimidine-5(4H)-one (I-150)

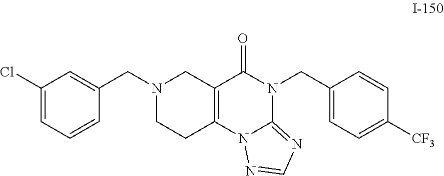 [SMILES: O=c1c2c(n3ncnc3n1Cc1ccc(C(F)(F)F)cc1)CCN(Cc1cccc(Cl)c1)C2]

The compound was prepared from compound 17-2 by referring to the synthetic method for compound I-147 in Example 220, MS (ESI) m/z: 474.2 [M+H]⁺.

### Example 224

### Preparation of 4-(4-trifluoromethylbenzyl)-7-(3-cyanobenzyl)-6,7,8,9-tetrahydropyrido[3,4-e][1,2,4]triazolo[1,5-a]pyrimidine-5(4H)-one (I-151)

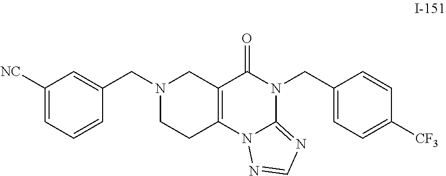 [SMILES: N#Cc1cccc(CN2CCc3c(c(=O)n(Cc4ccc(C(F)(F)F)cc4)c4ncnn34)C2)c1]

The compound was prepared from compound 17-2 by referring to the synthetic method for compound I-147 in Example 220, MS (ESI) m/z: 465.2 [M+H]⁺.

### Example 225

### Preparation of 4-(4-chlorobenzyl)-7-benzyl-6,7,8,9-tetrahydropyrido[3,4-e][1,2,4]triazolo[1,5-a]pyrimidine-5(4H)-one (I-152)

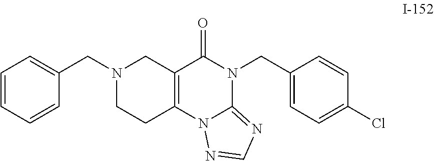 [SMILES: O=c1c2c(n3ncnc3n1Cc1ccc(Cl)cc1)CCN(Cc1ccccc1)C2]

The compound was prepared from compound 24-2 by referring to the synthetic method for compound I-148 in Example 221, MS (ESI) m/z: 406.2 [M+H]⁺.

### Example 226

### Preparation of 4-(4-chlorobenzyl)-7-(3-fluorobenzyl)-6,7,8,9-tetrahydropyrido[3,4-e][1,2,4]triazolo[1,5-a]pyrimidine-5(4H)-one (I-153)

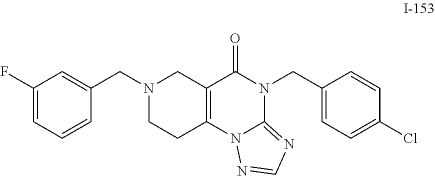 [SMILES: O=c1c2c(n3ncnc3n1Cc1ccc(Cl)cc1)CCN(Cc1cccc(F)c1)C2]

The compound was prepared from compound 24-2 by referring to the synthetic method for compound I-148 in Example 221, MS (ESI) m/z: 424.2 [M+H]⁺.

### Example 227

### Preparation of 4-(4-chlorobenzyl)-7-(3-chlorobenzyl)-6,7,8,9-tetrahydropyrido[3,4-e][1,2,4]triazolo[1,5-a]pyrimidine-5(4H)-one (I-154)

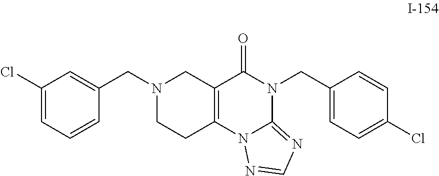 [SMILES: O=c1c2c(n3ncnc3n1Cc1ccc(Cl)cc1)CCN(Cc1cccc(Cl)c1)C2]

The compound was prepared from compound 24-2 by referring to the synthetic method for compound I-148 in Example 221, MS (ESI) m/z: 440.1 [M+H]⁺.

### Example 228

### Preparation of 4-(4-chlorobenzyl)-7-(3-cyanobenzyl)-6,7,8,9-tetrahydropyrido[3,4-e][1,2,4]triazolo[1,5-a]pyrimidine-5(4H)-one (I-155)

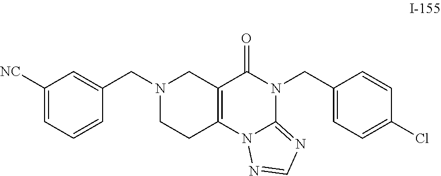 [SMILES: N#Cc1cccc(CN2CCc3c(c(=O)n(Cc4ccc(Cl)cc4)c4ncnn34)C2)c1]

The compound was prepared from compound 24-2 by referring to the synthetic method for compound I-148 in Example 221, MS (ESI) m/z: 431.2 [M+H]⁺.

### Example 229

### Preparation of 4-benzyl-7-(3-chlorobenzyl)-6,7,8,9-tetrahydropyrazolo[1,5-a]pyrido[3,4-e]pyrimidine-5(4H)-one (I-156)

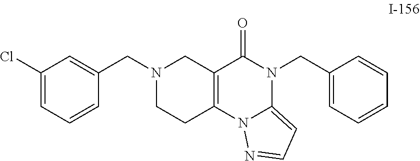 [SMILES: O=c1c2c(n3nccc3n1Cc1ccccc1)CCN(Cc1cccc(Cl)c1)C2]

The compound was prepared from compound 17-3 by referring to the synthetic method for compound I-147 in Example 220, MS (ESI) m/z: 405.2 [M+H]⁺.

### Preparation of 4-benzyl-7-(3-cyanobenzyl)-6,7,8,9-tetrahydropyrazolo[1,5-a]pyrido[3,4-e]pyrimidine-5(4H)-one (I-157)

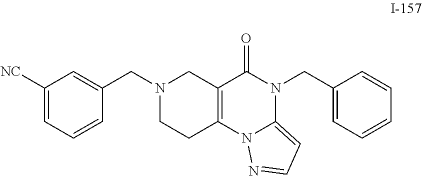 [SMILES: N#Cc1cccc(CN2CCc3c(c(=O)n(Cc4ccccc4)c4ccnn34)C2)c1]

The compound was prepared from compound 17-3 by referring to the synthetic method for compound I-147 in Example 220, MS (ESI) m/z: 396.2 [M+H]⁺.

### Example 231

### Preparation of 4-benzyl-7-(3-methylbenzyl)-6,7,8,9-tetrahydropyrazolo[1,5-a]pyrido[3,4-e]pyrimidine-5(4H)-one (I-158)

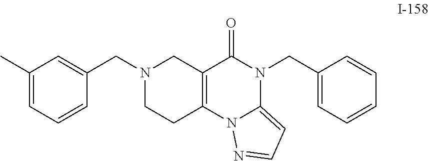 [SMILES: Cc1cccc(CN2CCc3c(c(=O)n(Cc4ccccc4)c4ccnn34)C2)c1]

The compound was prepared from compound 17-3 by referring to the synthetic method for compound I-147 in Example 220, MS (ESI) m/z: 385.2 [M+H]⁺.

### Example 232

### Preparation of 4-(4-trifluoromethylbenzyl)-7-benzyl-6,7,8,9-tetrahydropyrazolo[1,5-a]pyrido[3,4-e]pyrimidine-5(4H)-one (I-159)

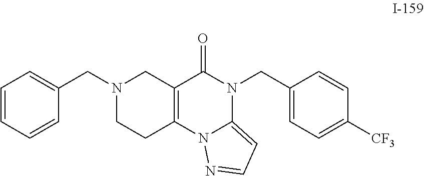 [SMILES: O=c1c2c(n3nccc3n1Cc1ccc(C(F)(F)F)cc1)CCN(Cc1ccccc1)C2]

The compound was prepared from compound 24-3 by referring to the synthetic method for compound I-148 in Example 221, MS (ESI) m/z: 439.2 [M+H]⁺.

### Example 233

### Preparation of 4-(4-trifluoromethylbenzyl)-7-(3-cyanobenzyl)-6,7,8,9-tetrahydropyrazolo[1,5-a]pyrido[3,4-e]pyrimidine-5(4H)-one (I-160)

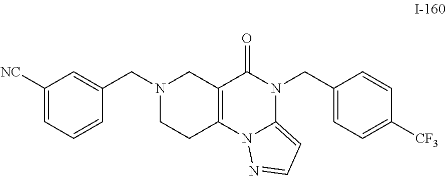 [SMILES: N#Cc1cccc(CN2CCc3c(c(=O)n(Cc4ccc(C(F)(F)F)cc4)c4ccnn34)C2)c1]

The compound was prepared from compound 24-3 by referring to the synthetic method for compound I-148 in Example 221, MS (ESI) m/z: 464.2 [M+H]⁺.

### Example 234

### Preparation of 4-(4-chlorobenzyl)-7-benzyl-6,7,8,9-tetrahydropyrazolo[1,5-a]pyrido[3,4-e]pyrimidine-5(4H)-one (I-161)

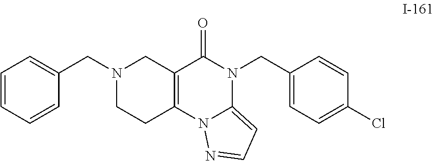 [SMILES: O=c1c2c(n3nccc3n1Cc1ccc(Cl)cc1)CCN(Cc1ccccc1)C2]

The compound was prepared from compound 24-3 by referring to the synthetic method for compound I-148 in Example 221, MS (ESI) m/z: 405.2 [M+H]⁺.

### Example 235

### Preparation of 4-(4-chlorobenzyl)-7-(3-chlorobenzyl)-6,7,8,9-tetrahydropyrazolo[1,5-a]pyrido[3,4-e]pyrimidine-5(4H)-one (I-162)

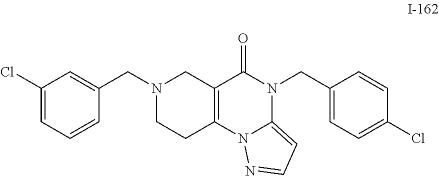 [SMILES: O=c1c2c(n3nccc3n1Cc1ccc(Cl)cc1)CCN(Cc1cccc(Cl)c1)C2]

The compound was prepared from compound 24-3 by referring to the synthetic method for compound I-148 in Example 221, MS (ESI) m/z: 439.1 [M+H]⁺.

### Example 236

### Preparation of 4-(4-chlorobenzyl)-7-(3-cyanobenzyl)-6,7,8,9-tetrahydropyrazolo[1,5-a]pyrido[3,4-e]pyrimidine-5(4H)-one (I-163)

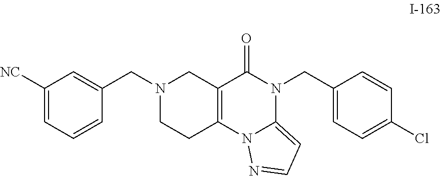 [SMILES: N#Cc1cccc(CN2CCc3c(c(=O)n(Cc4ccc(Cl)cc4)c4ccnn34)C2)c1]

The compound was prepared from compound 24-3 by referring to the synthetic method for compound I-148 in Example 221, MS (ESI) m/z: 430.2 [M+H]⁺.

### Example 237

### Preparation of 7-benzyl-4-(4-trifluoromethylbenzyl)-6,7,8,9-tetrahydroimidazo[1,5-a]pyrido[3,4-e]pyrimidine-5(4H)-one (I-164)

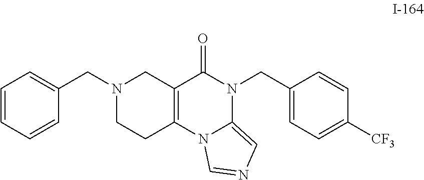 [SMILES: O=c1c2c(n3cncc3n1Cc1ccc(C(F)(F)F)cc1)CCN(Cc1ccccc1)C2]

The compound was prepared from compound 17-4 by referring to the synthetic method for compound I-147 in Example 220, MS (ESI) m/z: 439.2 [M+H]⁺.

### Example 238

### Preparation of 7-(3-cyanobenzyl)-4-(4-trifluoromethylbenzyl)-6,7,8,9-tetrahydroimidazo[1,5-a]pyrido[3,4-e]pyrimidine-5(4H)-one (I-165)

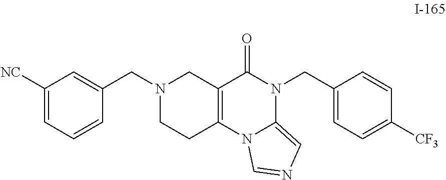 [SMILES: N#Cc1cccc(CN2CCc3c(c(=O)n(Cc4ccc(C(F)(F)F)cc4)c4cncn34)C2)c1]

The compound was prepared from compound 17-4 by referring to the synthetic method for compound I-147 in Example 220, MS (ESI) m/z: 464.2 [M+H]⁺.

### Example 239

### Preparation of 7-(3-chlorobenzyl)-4-(4-chlorobenzyl)-6,7,8,9-tetrahydroimidazo[1,5-a]pyrido[3,4-e]pyrimidine-5(4H)-one (I-166)

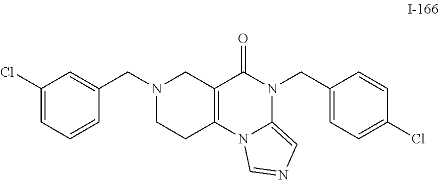 [SMILES: O=c1c2c(n3cncc3n1Cc1ccc(Cl)cc1)CCN(Cc1cccc(Cl)c1)C2]

The compound was prepared from compound 17-4 by referring to the synthetic method for compound I-147 in Example 220, MS (ESI) m/z: 439.1 [M+H]⁺.

### Example 240

### Preparation of 7-(3-cyanobenzyl)-4-(4-chlorobenzyl)-6,7,8,9-tetrahydroimidazo[1,5-a]pyrido[3,4-e]pyrimidine-5(4H)-one (I-167)

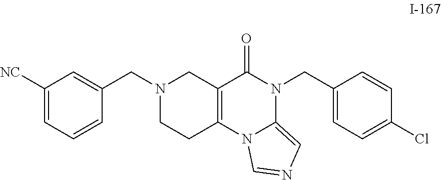 [SMILES: N#Cc1cccc(CN2CCc3c(c(=O)n(Cc4ccc(Cl)cc4)c4cncn34)C2)c1]

The compound was prepared from compound 17-4 by referring to the synthetic method for compound I-147 in Example 220, MS (ESI) m/z: 430.2 [M+H]⁺.

### Example 241

### Preparation of 3-benzyl-6-(4-trifluoromethylbenzyl)-1,2,3,4-tetrahydropyrido[3,4-e]pyrrolo[1,2-a]pyrimidine-5(6H)-one (I-168)

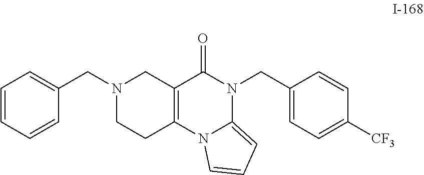 [SMILES: O=c1c2c(n3cccc3n1Cc1ccc(C(F)(F)F)cc1)CCN(Cc1ccccc1)C2]

The compound was prepared from compound 17-5 by referring to the synthetic method for compound I-147 in Example 220, MS (ESI) m/z: 438.1 [M+H]⁺.

### Example 242

### Preparation of 3-(3-chlorobenzyl)-6-(4-chlorobenzyl)-1,2,3,4-tetrahydropyrido[3,4-e]pyrrolo[1,2-a]pyrimidine-5(6H)-one (I-169)

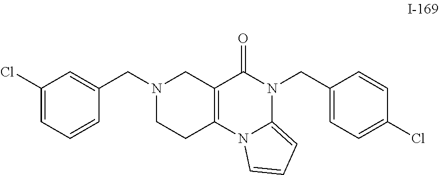 [SMILES: O=c1c2c(n3cccc3n1Cc1ccc(Cl)cc1)CCN(Cc1cccc(Cl)c1)C2]

The compound was prepared from compound 17-5 by referring to the synthetic method for compound I-147 in Example 220, MS (ESI) m/z: 438.2 [M+H]⁺.

### Example 243

The EC₅₀ values for activating ClpP hydrolysis of FITC-casein and the activity for inhibiting the growth of MV4; 11 cells of the compounds of formula I as ClpP agonists were determined by the methods as described in the literature (Cancer Cell 2019, 35, 721-737). The specific procedures were as follows:

### (1) Preparation of Compounds:

Test compounds were each prepared into a 100 mM stock solution in DMSO and stored. and 1 μL of stock solution of each of the compounds was taken and diluted to 10 mM by adding 9 μL of DMSO for later use.

### (2) Dilution of Compounds (2×, 50 μL/Well):

The compounds of formula I of the present invention were each diluted at concentrations of 200 μM, 50 μM, 10 μM, 2 μM, 500 nM, 100 nM, 20 nM, and 2 nM in an assay buffer. 50 μL of each of the compounds at different concentrations was added to each well, with 2 replicate wells. The final concentrations were 100 μM, 25 μM, 5 μM, 1 μM, 250 nM, 50 nM, 10 nM, and 1 nM.

### (3) Dilution of Protein (4×, 25 μL/Well):

The protein hClpP purified by referring to the method in the literature (Cancer Cell 2019, 35, 721-737.) was at a concentration of 14 mg/mL, which was converted to a concentration of 1000 μM. The protein was diluted to 4 μM in an assay buffer. 25 μL of protein was added to each well, and incubated at 37° C. for 30 min. The final concentration was 1 μM.

### (4) Dilution of FITC-Casein (4×, 25 μL/Well)

A FITC-Casein stock solution labeled by referring to the method in the literature (Cancer Cell 2019, 35, 721-737) was at 200 mM, which was diluted to 16 μM in an assay buffer. 25 μL of FITC-Casein was added to each well. The final concentration was 4 μM. The mixture was immediately loaded on the machine for detection.

### (5) Arrangement of Control Group

FITC-Casein and FITC-Casein+ClpP were used as negative controls. The positive drugs ONC201 and ONC212 were used as positive control groups, and the positive drugs ONC201 and ONC212 were synthesized by the method in the literature (Angewandte Chemie International Edition 2014, 53, 6628-6631).

### (6) Fluorescence Kinetic Detection

The detection temperature was 37° C. The plate was shaken for 3 s. The excitation light was at the wavelength of 485±20 nm, and the emission light was at the wavelength of 525±20 nm. Kinetic parameters were set, and scanning was performed once every 1 min for 15 min.

The results for the EC₅₀ values for activating ClpP hydrolysis of FITC-casein and the activity for inhibiting the growth of MV4; 11 cells of the compounds of formula I as ClpP agonists were shown in the table below.

| | ClpP | MV4; 11 |
|---|---|---|
| Compound | EC₅₀ (μM) | IC₅₀ (μM) |
| ONC201 | 1~10 | 0.1~10 |
| I-1 | 10~100 | 1~10 |
| I-2 | 10~100 | 1~10 |
| I-3 | 10~100 | 1~10 |
| I-4 | 10~100 | <1 |
| I-5 | 1~10 | <1 |
| I-6 | 1~10 | <1 |
| I-7 | 1~10 | <1 |
| I-8 | 1~10 | <1 |
| I-9 | 1~10 | <1 |
| I-10 | 1~10 | <1 |
| I-11 | 1~10 | <1 |
| I-12 | <1 | <1 |
| I-13 | <1 | <1 |
| I-14 | <1 | <1 |
| I-15 | <1 | <1 |
| I-16 | 1~10 | <1 |
| I-17 | 1~10 | <1 |
| I-18 | 1~10 | <1 |
| I-19 | 1~10 | <1 |
| I-20 | 1~10 | <1 |
| I-21 | 1~10 | <1 |
| I-22 | 1~10 | <1 |
| I-23 | 1~10 | <1 |
| I-24 | 10~100 | 1~10 |
| I-25 | 10~100 | 1~10 |
| I-26 | 10~100 | 1~10 |
| I-27 | 10~100 | 1~10 |
| I-28 | 10~100 | 1~10 |
| I-29 | 10~100 | 1~10 |
| I-30 | 10~100 | 1~10 |
| I-31 | 10~100 | 1~10 |
| I-32 | 10~100 | 1~10 |
| I-33 | 10~100 | 1~10 |
| I-34 | 10~100 | 1~10 |
| I-35 | 10~100 | 1~10 |
| I-36 | 10~100 | 1~10 |
| I-37 | 10~100 | 1~10 |
| I-38 | 10~100 | 1~10 |
| I-39 | 10~100 | 0.91 |
| I-40 | 1~10 | <1 |
| I-41 | 1~10 | <1 |
| I-42 | 1~10 | <1 |
| I-43 | 1~10 | <1 |
| I-44 | 1~10 | <1 |
| I-45 | 1~10 | <1 |
| I-46 | <1 | <1 |
| I-47 | <1 | <1 |
| I-48 | <1 | <1 |
| I-49 | <1 | <1 |
| I-50 | <1 | <1 |
| I-51 | <1 | <1 |
| I-52 | <1 | <1 |
| I-53 | <1 | <1 |
| I-54 | <1 | <1 |
| I-55 | <1 | <1 |
| I-56 | <1 | <1 |
| I-57 | <1 | <1 |
| I-58 | 10~100 | 1~10 |
| I-59 | 10~100 | 1~10 |
| I-60 | 10~100 | 1~10 |
| I-61 | 10~100 | 1~10 |
| I-62 | 10~100 | 1~10 |
| I-63 | 10~100 | 1~10 |
| I-64 | 10~100 | 1~10 |
| I-65 | 10~100 | 1~10 |
| I-66 | 10~100 | 1~10 |
| I-67 | 10~100 | 1~10 |
| I-68 | 10~100 | 1~10 |
| I-69 | 10~100 | 1~10 |
| I-70 | <1 | <1 |
| I-71 | <1 | <1 |
| I-72 | <1 | <1 |
| I-73 | <1 | <1 |
| I-74 | <1 | <1 |
| I-75 | <1 | <1 |
| I-76 | <1 | <1 |
| I-77 | <1 | <1 |
| I-78 | <1 | <1 |
| I-79 | <1 | <1 |
| I-80 | <1 | <1 |
| I-81 | <1 | <1 |
| I-82 | <1 | <1 |
| I-83 | <1 | <1 |
| I-84 | <1 | <1 |
| I-85 | <1 | <1 |
| I-86 | <1 | <1 |
| I-87 | <1 | <1 |
| I-88 | 1~10 | 1~10 |
| I-89 | 1~10 | 1~10 |
| I-90 | 1~10 | 1~10 |
| I-91 | <1 | <1 |
| I-92 | <1 | <1 |
| I-93 | <1 | <1 |
| I-94 | <1 | <1 |
| I-95 | <1 | <1 |
| I-96 | <1 | <1 |
| I-97 | 1~10 | 1~10 |
| I-98 | 1~10 | 1~10 |
| I-99 | <1 | <1 |
| I-100 | <1 | <1 |
| I-101 | <1 | <1 |
| I-102 | <1 | <1 |
| I-103 | <1 | <1 |
| I-104 | <1 | <1 |
| I-105 | 1~10 | 1~10 |
| I-106 | 1~10 | 1~10 |
| I-107 | 1~10 | 1~10 |
| I-108 | <1 | <1 |
| I-109 | <1 | <1 |
| I-110 | <1 | <1 |
| I-111 | <1 | <1 |
| I-112 | <1 | <1 |
| I-113 | <1 | <1 |
| I-114 | <1 | <1 |
| I-115 | <1 | <1 |
| I-116 | <1 | <1 |
| I-117 | <1 | <1 |
| I-118 | <1 | <1 |
| I-119 | <1 | <1 |
| I-120 | <1 | <1 |
| I-121 | <1 | <1 |
| I-122 | <1 | <1 |
| I-123 | <1 | <1 |
| I-124 | <1 | <1 |
| I-125 | <1 | <1 |
| I-126 | <1 | <1 |
| I-127 | <1 | <1 |
| I-128 | <1 | <1 |
| I-129 | <1 | <1 |
| I-130 | <1 | <1 |
| I-131 | <1 | <1 |
| I-132 | <1 | <1 |
| I-133 | <1 | <1 |
| I-134 | <1 | <1 |
| I-135 | <1 | <1 |
| I-136 | <1 | <1 |
| I-137 | <1 | <1 |
| I-138 | <1 | <1 |
| I-139 | <1 | <1 |
| I-140 | <1 | <1 |
| I-141 | <1 | <1 |
| I-142 | <1 | <1 |
| I-143 | <1 | <1 |
| I-144 | <1 | <1 |
| I-145 | <1 | <1 |
| I-146 | <1 | <1 |
| I-147 | 1~10 | 1~10 |
| I-148 | 1~10 | 1~10 |
| I-149 | 1~10 | 1~10 |
| I-150 | 1~10 | 1~10 |
| I-151 | 1~10 | 1~10 |
| I-152 | 1~10 | 1~10 |
| I-153 | 1~10 | 1~10 |
| I-154 | 1~10 | 1~10 |
| I-155 | 1~10 | 1~10 |
| I-156 | 1~10 | 1~10 |
| I-157 | 1~10 | 1~10 |
| I-158 | 1~10 | 1~10 |
| I-159 | 1~10 | 1~10 |
| I-160 | 1~10 | 1~10 |
| I-161 | 1~10 | 1~10 |
| I-162 | 1~10 | 1~10 |
| I-163 | 1~10 | 1~10 |
| I-164 | 1~10 | 1~10 |
| I-165 | 1~10 | 1~10 |
| I-166 | 1~10 | 1~10 |
| I-167 | 1~10 | 1~10 |
| I-168 | <1 | <1 |
| I-169 | <1 | <1 |

1. Compounds containing a tetrahydronaphthyridinone or tetrahydropyridopyrimidinone skeleton, having a structural general formula shown in formula I: 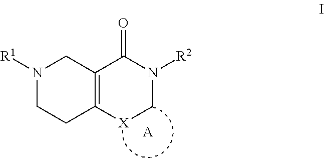
wherein, X is a carbon atom or a nitrogen atom, and ring A is a substituted or unsubstituted aromatic ring or a substituted or unsubstituted aromatic heterocyclic ring;
R¹ is selected from alkyl, substituted alkyl, cycloalkyl, substituted cycloalkyl, optionally substituted aryl, optionally substituted heterocycloaryl, aralkyl, optionally substituted aralkyl, heterocycloaralkyl, and optionally substituted heterocycloaralkyl; R² is selected from alkyl, substituted alkyl, cycloalkyl, substituted cycloalkyl, optionally substituted aryl, optionally substituted heterocycloaryl, aralkyl, optionally substituted aralkyl, heterocycloaralkyl, and optionally substituted heterocycloaralkyl.

2. The compounds containing a tetrahydronaphthyridinone or tetrahydropyridopyrimidinone skeleton according to claim 1, wherein the ring A is a benzene ring, a pyridine ring, a pyrimidine ring, an imidazole ring, a thiophene ring, a furan ring, a thiazole ring, a triazazole ring, a pyrazole ring, or a pyrrole ring.

3. The compounds containing a tetrahydronaphthyridinone or tetrahydropyridopyrimidinone skeleton according to claim 1, wherein R¹ is selected from cyclopropylmethyl, cyclobutylmethyl, cyclopentylmethyl, cyclohexylmethyl, benzyl, 2-fluorobenzyl, 2-chlorobenzyl, 2-bromobenzyl, 2-methylbenzyl, 2-ethylbenzyl, 2-methoxybenzyl, 3-fluorobenzyl, 3-chlorobenzyl, 3-bromobenzyl, 3-methylbenzyl, 3-ethylbenzyl, 3-methoxybenzyl, 3-cyanobenzyl, 4-fluorobenzyl, 4-chlorobenzyl, 4-bromobenzyl, 4-methylbenzyl, 4-ethylbenzyl, 4-methoxybenzyl, 4-trifluoromethylbenzyl, (1-methyl-1H-pyrazol-3-yl)methyl, (1-methyl-1H-pyrazol-5-yl)methyl, (1,3-dimethyl-1H-pyrazol-5-yl)methyl, pyridin-2-ylmethyl, pyridin-3-ylmethyl, pyridin-4-ylmethyl, (4-chloropyridin-2-yl)methyl, (1-methyl-1H-imidazol-2-yl)methyl, (1-methyl-1H-imidazol-4-yl)methyl, (1-methyl-1H-imidazol-5-yl)methyl, (3,5-dimethylisoxazol-4-yl)methyl, (2-methylthiazol-5-yl)methyl, and benzo[1,3]dioxol-4-ylmethyl.

4. The compounds containing a tetrahydronaphthyridinone or tetrahydropyridopyrimidinone skeleton according to claim 1, wherein R² is selected from cyclopropylmethyl, cyclobutylmethyl, cyclopentylmethyl, cyclohexylmethyl, benzyl, 2-fluorobenzyl, 2-chlorobenzyl, 2-bromobenzyl, 2-methylbenzyl, 2-ethylbenzyl, 2-methoxybenzyl, 3-fluorobenzyl, 3-chlorobenzyl, 3-bromobenzyl, 3-methylbenzyl, 3-ethylbenzyl, 3-methoxybenzyl, 4-fluorobenzyl, 4-chlorobenzyl, 4-bromobenzyl, 4-methylbenzyl, 4-ethylbenzyl, 4-methoxybenzyl, 4-nitrobenzyl, 4-methylaminobenzyl, 4-dimethylaminobenzyl, 4-trifluoromethylbenzyl, 2,4-difluorobenzyl, 2-fluoro-4-chlorobenzyl, 2-fluoro-4-methylbenzyl, 2-fluoro-4-methoxybenzyl, 2-fluoro-4-trifluoromethylbenzyl, 2-fluoro-4-bromobenzyl, (1-methyl-1H-pyrazol-3-yl)methyl, (1-methyl-1H-pyrazol-5-yl)methyl, (1,3-dimethyl-1H-pyrazol-5-yl)methyl, pyridin-2-ylmethyl, pyridin-3-ylmethyl, pyridin-4-ylmethyl, (4-chloropyridin-2-yl)methyl, (1-methyl-1H-imidazol-2-yl)methyl, (1-methyl-1H-imidazol-4-yl)methyl, (1-methyl-1H-imidazol-5-yl)methyl, (3,5-dimethylisoxazol-4-yl)methyl, (2-methylthiazol-5-yl)methyl, benzo[1,3]dioxol-4-ylmethyl, benzo[1,3]dioxol-5-ylmethyl, benzo[1,4]dioxan-5-ylmethyl, and [1,4]dioxan-6-ylmethyl.

5. The compounds containing a tetrahydronaphthyridinone or tetrahydropyridopyrimidinone skeleton according to claim 1, wherein the compounds are selected from the following compounds:
6-benzyl-3-(cyclopropylmethyl)-2,3,4,6-tetrahydropyrido[3,4-c][1,8]naphthyridine-5-(1H)-one;
6-benzyl-3-(cyclobutylmethyl)-2,3,4,6-tetrahydropyrido[3,4-c][1,8]naphthyridine-5-(1H)-one;
6-benzyl-3-(cyclopentylmethyl)-2,3,4,6-tetrahydropyrido[3,4-c][1,8]naphthyridine-5-(1H)-one;
6-benzyl-3-(cyclohexylmethyl)-2,3,4,6-tetrahydropyrido[3,4-c][1,8]naphthyridine-5-(1H)-one;
3,6-dibenzyl-2,3,4,6-tetrahydropyrido[3,4-c][1,8]naphthyridine-5-(1H)-one;
6-benzyl-3-(2-fluorobenzyl)-2,3,4,6-tetrahydropyrido[3,4-c][1,8]naphthyridine-5-(1H)-one;
6-benzyl-3-(2-chlorobenzyl)-2,3,4,6-tetrahydropyrido[3,4-c][1,8]naphthyridine-5-(1H)-one;
6-benzyl-3-(2-bromobenzyl)-2,3,4,6-tetrahydropyrido[3,4-c][1,8]naphthyridine-5-(1H)-one;
6-benzyl-3-(2-methylbenzyl)-2,3,4,6-tetrahydropyrido[3,4-c][1,8]naphthyridine-5-(1H)-one;
6-benzyl-3-(2-ethylbenzyl)-2,3,4,6-tetrahydropyrido[3,4-c][1,8]naphthyridine-5-(1H)-one;
6-benzyl-3-(2-methoxybenzyl)-2,3,4,6-tetrahydropyrido[3,4-c][1,8]naphthyridine-5-(1H)-one;
6-benzyl-3-(3-fluorobenzyl)-2,3,4,6-tetrahydropyrido[3,4-c][1,8]naphthyridine-5-(1H)-one;
6-benzyl-3-(3-chlorobenzyl)-2,3,4,6-tetrahydropyrido[3,4-c][1,8]naphthyridine-5-(1H)-one;
6-benzyl-3-(3-bromobenzyl)-2,3,4,6-tetrahydropyrido[3,4-c][1,8]naphthyridine-5-(1H)-one;
6-benzyl-3-(3-methylbenzyl)-2,3,4,6-tetrahydropyrido[3,4-c][1,8]naphthyridine-5-(1H)-one;
6-benzyl-3-(3-ethylbenzyl)-2,3,4,6-tetrahydropyrido[3,4-c][1,8]naphthyridine-5-(1H)-one;
6-benzyl-3-(3-methoxybenzyl)-2,3,4,6-tetrahydropyrido[3,4-c][1,8]naphthyridine-5-(1H)-one;
6-benzyl-3-(4-fluorobenzyl)-2,3,4,6-tetrahydropyrido[3,4-c][1,8]naphthyridine-5-(1H)-one;
6-benzyl-3-(4-chlorobenzyl)-2,3,4,6-tetrahydropyrido[3,4-c][1,8]naphthyridine-5-(1H)-one;
6-benzyl-3-(4-bromobenzyl)-2,3,4,6-tetrahydropyrido[3,4-c][1,8]naphthyridine-5-(1H)-one;
6-benzyl-3-(4-methylbenzyl)-2,3,4,6-tetrahydropyrido[3,4-c][1,8]naphthyridine-5-(1H)-one;
6-benzyl-3-(4-ethylbenzyl)-2,3,4,6-tetrahydropyrido[3,4-c][1,8]naphthyridine-5-(1H)-one;
6-benzyl-3-(4-methoxybenzyl)-2,3,4,6-tetrahydropyrido[3,4-c][1,8]naphthyridine-5-(1H)-one;
6-benzyl-3-((1-methyl-1H-pyrazol-3-yl)methyl)-2,3,4,6-tetrahydropyrido[3,4-c][1,8]naphthyridine-5-(1H)-one;
6-benzyl-3-((1-methyl-1H-pyrazol-5-yl)methyl)-2,3,4,6-tetrahydropyrido[3,4-c][1,8]naphthyridine-5-(1H)-one;
6-benzyl-3-((1,3-dimethyl-1H-pyrazol-5-yl)methyl)-2,3,4,6-tetrahydropyrido[3,4-c][1,8]naphthyridine-5-(1H)-one;
6-benzyl-3-(pyridin-2-ylmethyl)-2,3,4,6-tetrahydropyrido[3,4-c][1,8]naphthyridine-5-(1H)-one;
6-benzyl-3-(pyridin-3-ylmethyl)-2,3,4,6-tetrahydropyrido[3,4-c][1,8]naphthyridine-5-(1H)-one;
6-benzyl-3-(pyridin-4-ylmethyl)-2,3,4,6-tetrahydropyrido[3,4-c][1,8]naphthyridine-5-(1H)-one;
6-benzyl-3-((4-chloropyridin-2-yl)methyl)-2,3,4,6-tetrahydropyrido[3,4-c][1,8]naphthyridine-5-(1H)-one;
6-benzyl-3-((1-methyl-1H-imidazol-2-yl)methyl)-2,3,4,6-tetrahydropyrido[3,4-c][1,8]naphthyridine-5-(1H)-one;
6-benzyl-3-((1-methyl-1H-imidazol-4-yl)methyl)-2,3,4,6-tetrahydropyrido[3,4-c][1,8]naphthyridine-5-(1H)-one;
6-benzyl-3-((1-methyl-1H-imidazol-5-yl)methyl)-2,3,4,6-tetrahydropyrido[3,4-c][1,8]naphthyridine-5-(1H)-one;
6-benzyl-3-((3,5-dimethylisoxazol-4-yl)methyl)-2,3,4,6-tetrahydropyrido[3,4-c][1,8]naphthyridine-5-(1H)-one;
6-benzyl-3-((2-methylthiazol-5-yl)methyl)-2,3,4,6-tetrahydropyrido[3,4-c][1,8]naphthyridine-5-(1H)-one;
3-benzyl-6-(cyclopropylmethyl)-2,3,4,6-tetrahydropyrido[3,4-c][1,8]naphthyridine-5-(1H)-one;
3-benzyl-6-(cyclobutylmethyl)-2,3,4,6-tetrahydropyrido[3,4-c][1,8]naphthyridine-5-(1H)-one;
3-benzyl-6-(cyclopentylmethyl)-2,3,4,6-tetrahydropyrido[3,4-c][1,8]naphthyridine-5-(1H)-one;
3-benzyl-6-(cyclohexylmethyl)-2,3,4,6-tetrahydropyrido[3,4-c][1,8]naphthyridine-5-(1H)-one;
3-benzyl-6-(2-fluorobenzyl)-2,3,4,6-tetrahydropyrido[3,4-c][1,8]naphthyridine-5-(1H)-one;
3-benzyl-6-(2-chlorobenzyl)-2,3,4,6-tetrahydropyrido[3,4-c][1,8]naphthyridine-5-(1H)-one;
3-benzyl-6-(2-bromobenzyl)-2,3,4,6-tetrahydropyrido[3,4-c][1,8]naphthyridine-5-(1H)-one;
3-benzyl-6-(2-methylbenzyl)-2,3,4,6-tetrahydropyrido[3,4-c][1,8]naphthyridine-5-(1H)-one;
3-benzyl-6-(2-ethylbenzyl)-2,3,4,6-tetrahydropyrido[3,4-c][1,8]naphthyridine-5-(1H)-one;
3-benzyl-6-(2-methoxybenzyl)-2,3,4,6-tetrahydropyrido[3,4-c][1,8]naphthyridine-5-(1H)-one;
3-benzyl-6-(3-fluorobenzyl)-2,3,4,6-tetrahydropyrido[3,4-c][1,8]naphthyridine-5-(1H)-one;
3-benzyl-6-(3-chlorobenzyl)-2,3,4,6-tetrahydropyrido[3,4-c][1,8]naphthyridine-5-(1H)-one;
3-benzyl-6-(3-bromobenzyl)-2,3,4,6-tetrahydropyrido[3,4-c][1,8]naphthyridine-5-(1H)-one;
3-benzyl-6-(3-methylbenzyl)-2,3,4,6-tetrahydropyrido[3,4-c][1,8]naphthyridine-5-(1H)-one;
3-benzyl-6-(3-ethylbenzyl)-2,3,4,6-tetrahydropyrido[3,4-c][1,8]naphthyridine-5-(1H)-one;
3-benzyl-6-(3-methoxybenzyl)-2,3,4,6-tetrahydropyrido[3,4-c][1,8]naphthyridine-5-(1H)-one;
3-benzyl-6-(4-fluorobenzyl)-2,3,4,6-tetrahydropyrido[3,4-c][1,8]naphthyridine-5-(1H)-one;
3-benzyl-6-(4-chlorobenzyl)-2,3,4,6-tetrahydropyrido[3,4-c][1,8]naphthyridine-5-(1H)-one;
3-benzyl-6-(4-bromobenzyl)-2,3,4,6-tetrahydropyrido[3,4-c][1,8]naphthyridine-5-(1H)-one;
3-benzyl-6-(4-methylbenzyl)-2,3,4,6-tetrahydropyrido[3,4-c][1,8]naphthyridine-5-(1H)-one;
3-benzyl-6-(4-ethylbenzyl)-2,3,4,6-tetrahydropyrido[3,4-c][1,8]naphthyridine-5-(1H)-one;
3-benzyl-6-(4-methoxybenzyl)-2,3,4,6-tetrahydropyrido[3,4-c][1,8]naphthyridine-5-(1H)-one;
3-benzyl-6-((1-methyl-1H-pyrazol-3-yl)methyl)-2,3,4,6-tetrahydropyrido[3,4-c][1,8]naphthyridine-5-(1H)-one;
3-benzyl-6-((1-methyl-1H-pyrazol-5-yl)methyl)-2,3,4,6-tetrahydropyrido[3,4-c][1,8]naphthyridine-5-(1H)-one;
3-benzyl-6-((1,3-dimethyl-1H-pyrazol-5-yl)methyl)-2,3,4,6-tetrahydropyrido[3,4-c][1,8]naphthyridine-5-(1H)-one;
3-benzyl-6-(pyridin-2-ylmethyl)-2,3,4,6-tetrahydropyrido[3,4-c][1,8]naphthyridine-5-(1H)-one;
3-benzyl-6-(pyridin-3-ylmethyl)-2,3,4,6-tetrahydropyrido[3,4-c][1,8]naphthyridine-5-(1H)-one;
3-benzyl-6-(pyridin-4-ylmethyl)-2,3,4,6-tetrahydropyrido[3,4-c][1,8]naphthyridine-5-(1H)-one;
3-benzyl-6-((4-chloropyridin-2-yl)methyl)-2,3,4,6-tetrahydropyrido[3,4-c][1,8]naphthyridine-5-(1H)-one;
3-benzyl-6-((1-methyl-1H-imidazol-2-yl)methyl)-2,3,4,6-tetrahydropyrido[3,4-c][1,8]naphthyridine-5-(1H)-one;
3-benzyl-6-((1-methyl-1H-imidazol-4-yl)methyl)-2,3,4,6-tetrahydropyrido[3,4-c][1,8]naphthyridine-5-(1H)-one;
3-benzyl-6-((1-methyl-1H-imidazol-5-yl)methyl)-2,3,4,6-tetrahydropyrido[3,4-c][1,8]naphthyridine-5-(1H)-one;
3-benzyl-6-((3,5-dimethylisoxazol-4-yl)methyl)-2,3,4,6-tetrahydropyrido[3,4-c][1,8]naphthyridine-5-(1H)-one;
3-benzyl-6-((2-methylthiazol-5-yl)methyl)-2,3,4,6-tetrahydropyrido[3,4-c][1,8]naphthyridine-5-(1H)-one;
3-benzyl-6-(4-(trifluoromethyl)benzyl)-2,3,4,6-tetrahydropyrido[3,4-c][1,8]naphthyridine-5-(1H)-one;
3-(3-fluorobenzyl)-6-(4-fluorobenzyl)-2,3,4,6-tetrahydropyrido[3,4-c][1,8]naphthyridine-5-(1H)-one;
3-(3-chlorobenzyl)-6-(4-fluorobenzyl)-2,3,4,6-tetrahydropyrido[3,4-c][1,8]naphthyridine-5-(1H)-one;
3-(3-bromobenzyl)-6-(4-fluorobenzyl)-2,3,4,6-tetrahydropyrido[3,4-c][1,8]naphthyridine-5-(1H)-one;
3-(3-fluorobenzyl)-6-(4-chlorobenzyl)-2,3,4,6-tetrahydropyrido[3,4-c][1,8]naphthyridine-5-(1H)-one;
3-(3-chlorobenzyl)-6-(4-chlorobenzyl)-2,3,4,6-tetrahydropyrido[3,4-c][1,8]naphthyridine-5-(1H)-one;
3-(3-bromobenzyl)-6-(4-chlorobenzyl)-2,3,4,6-tetrahydropyrido[3,4-c][1,8]naphthyridine-5-(1H)-one;
3-(3-fluorobenzyl)-6-(4-bromobenzyl)-2,3,4,6-tetrahydropyrido[3,4-c][1,8]naphthyridine-5-(1H)-one;
3-(3-chlorobenzyl)-6-(4-bromobenzyl)-2,3,4,6-tetrahydropyrido[3,4-c][1,8]naphthyridine-5-(1H)-one;
3-(3-bromobenzyl)-6-(4-bromobenzyl)-2,3,4,6-tetrahydropyrido[3,4-c][1,8]naphthyridine-5-(1H)-one;
3-benzyl-6-(4-(trifluoromethyl)benzyl)-2,3,4,6-tetrahydrobenzo[c][2,7]naphthyridine-5-(1H)-one;
8-benzyl-5-(4-(trifluoromethyl)benzyl)-7,8,9,10-tetrahydropyrimidinyl[4,5-c][2,7]naphthyridine-6-(5H)-one;
6-benzyl-3-(3-cyanobenzyl)-2,3,4,6-tetrahydropyrido[3,4-c][1,8]naphthyridine-5-(1H)-one;
6-(4-trifluoromethylbenzyl)-3-(3-cyanobenzyl)-2,3,4,6-tetrahydropyrido[3,4-c][1,8]naphthyridine-5-(1H)-one;
6-(4-fluorobenzyl)-3-(3-cyanobenzyl)-2,3,4,6-tetrahydropyrido[3,4-c][1,8]naphthyridine-5-(1H)-one;
6-(4-chlorobenzyl)-3-(3-cyanobenzyl)-2,3,4,6-tetrahydropyrido[3,4-c][1,8]naphthyridine-5-(1H)-one;
6-(4-bromobenzyl)-3-(3-cyanobenzyl)-2,3,4,6-tetrahydropyrido[3,4-c][1,8]naphthyridine-5-(1H)-one;
6-(4-methoxybenzyl)-3-(3-cyanobenzyl)-2,3,4,6-tetrahydropyrido[3,4-c][1,8]naphthyridine-5-(1H)-one;
6-(4-nitrobenzyl)-3-(3-cyanobenzyl)-2,3,4,6-tetrahydropyrido[3,4-c][1,8]naphthyridine-5-(1H)-one;
6-(4-methylaminobenzyl)-3-(3-cyanobenzyl)-2,3,4,6-tetrahydropyrido[3,4-c][1,8]naphthyridine-5-(1H)-one;
6-(4-dimethylaminobenzyl)-3-(3-cyanobenzyl)-2,3,4,6-tetrahydropyrido[3,4-c][1,8]naphthyridine-5-(1H)-one;
6-(4-methylbenzyl)-3-(3-cyanobenzyl)-2,3,4,6-tetrahydropyrido[3,4-c][1,8]naphthyridine-5-(1H)-one;
6-(4-methylbenzyl)-3-(3-chlorobenzyl)-2,3,4,6-tetrahydropyrido[3,4-c][1,8]naphthyridine-5-(1H)-one;
6-(4-methylbenzyl)-3-(3-fluorobenzyl)-2,3,4,6-tetrahydropyrido[3,4-c][1,8]naphthyridine-5-(1H)-one;
6-(4-ethylbenzyl)-3-(3-cyanobenzyl)-2,3,4,6-tetrahydropyrido[3,4-c][1,8]naphthyridine-5-(1H)-one;
6-(4-ethylbenzyl)-3-(3-fluorobenzyl)-2,3,4,6-tetrahydropyrido[3,4-c][1,8]naphthyridine-5-(1H)-one;
6-(4-ethylbenzyl)-3-(3-chlorobenzyl)-2,3,4,6-tetrahydropyrido[3,4-c][1,8]naphthyridine-5-(1H)-one;
3-(3-chlorobenzyl)-6-((2,3-dihydro[1,4]benzodioxin-6-yl)methyl)-2,3,4,6-tetrahydropyrido[3,4-c][1,8]naphthyridine-5(1H)-one;
6-([1,3]benzodioxol-5-ylmethyl)-3-(3-cyanobenzyl)-2,3,4,6-tetrahydropyrido[3,4-c][1,8]naphthyridine-5-(1H)-one;
6-(4-methoxybenzyl)-3-(3-fluorobenzyl)-2,3,4,6-tetrahydropyrido[3,4-c][1,8]naphthyridine-5-(1H)-one;
6-(4-methoxybenzyl)-3-(3-chlorobenzyl)-2,3,4,6-tetrahydropyrido[3,4-c][1,8]naphthyridine-5-(1H)-one;
7-benzyl-4-(4-trifluoromethylbenzyl)-6,7,8,9-tetrahydrofuro[3,4-c][2,7]naphthyridine-5(4H)-one;
7-benzyl-4-(4-trifluoromethylbenzyl)-6,7,8,9-tetrahydrothieno[3,4-c][2,7]naphthyridine-5(4H)-one;
7-benzyl-4-(4-trifluoromethylbenzyl)-6,7,8,9-tetrahydrothieno[3,2-c][2,7]naphthyridine-5(4H)-one;
7-benzyl-4-(4-trifluoromethylbenzyl)-6,7,8,9-tetrahydrothieno[2,3-c][2,7]naphthyridine-5(4H)-one;
7-benzyl-4-(4-trifluoromethylbenzyl)-6,7,8,9-tetrahydrothiazolo[4,5-c][2,7]naphthyridine-5(4H)-one;
7-benzyl-4-(2-methylbenzyl)-6,7,8,9-tetrahydroimidazo[1,2-a]pyrido[3,4-e]pyrimidine-5(4H)-one;
4,7-dibenzyl-6,7,8,9-tetrahydroimidazo[1,2-a]pyrido[3,4-e]pyrimidine-5(4H)-one;
7-(3-fluorobenzyl)-4-benzyl-6,7,8,9-tetrahydroimidazo[1,2-a]pyrido[3,4-e]pyrimidine-5(4H)-one;
7-(3-chlorobenzyl)-4-benzyl-6,7,8,9-tetrahydroimidazo[1,2-a]pyrido[3,4-e]pyrimidine-5(4H)-one;
7-(3-methylbenzyl)-4-benzyl-6,7,8,9-tetrahydroimidazo[1,2-a]pyrido[3,4-e]pyrimidine-5(4H)-one;
7-(3-cyanobenzyl)-4-benzyl-6,7,8,9-tetrahydroimidazo[1,2-a]pyrido[3,4-e]pyrimidine-5(4H)-one;
7-benzyl-4-(4-fluorobenzyl)-6,7,8,9-tetrahydroimidazo[1,2-a]pyrido[3,4-e]pyrimidine-5(4H)-one;
7-(3-fluorobenzyl)-4-(4-fluorobenzyl)-6,7,8,9-tetrahydroimidazo[1,2-a]pyrido[3,4-e]pyrimidine-5(4H)-one;
7-(3-chlorobenzyl)-4-(4-fluorobenzyl)-6,7,8,9-tetrahydroimidazo[1,2-a]pyrido[3,4-e]pyrimidine-5(4H)-one;
7-(3-methylbenzyl)-4-(4-fluorobenzyl)-6,7,8,9-tetrahydroimidazo[1,2-a]pyrido[3,4-e]pyrimidine-5(4H)-one;
7-(3-cyanobenzyl)-4-(4-fluorobenzyl)-6,7,8,9-tetrahydroimidazo[1,2-a]pyrido[3,4-e]pyrimidine-5(4H)-one;
7-benzyl-4-(4-chlorobenzyl)-6,7,8,9-tetrahydroimidazo[1,2-a]pyrido[3,4-e]pyrimidine-5(4H)-one;
7-(3-fluorobenzyl)-4-(4-chlorobenzyl)-6,7,8,9-tetrahydroimidazo[1,2-a]pyrido[3,4-e]pyrimidine-5(4H)-one;
7-(3-chlorobenzyl)-4-(4-chlorobenzyl)-6,7,8,9-tetrahydroimidazo[1,2-a]pyrido[3,4-e]pyrimidine-5(4H)-one;
7-(3-methylbenzyl)-4-(4-chlorobenzyl)-6,7,8,9-tetrahydroimidazo[1,2-a]pyrido[3,4-e]pyrimidine-5(4H)-one;
7-(3-cyanobenzyl)-4-(4-chlorobenzyl)-6,7,8,9-tetrahydroimidazo[1,2-a]pyrido[3,4-e]pyrimidine-5(4H)-one;
7-benzyl-4-(2,4-difluorobenzyl)-6,7,8,9-tetrahydroimidazo[1,2-a]pyrido[3,4-e]pyrimidine-5(4H)-one;
7-(3-fluorobenzyl)-4-(2,4-difluorobenzyl)-6,7,8,9-tetrahydroimidazo[1,2-a]pyrido[3,4-e]pyrimidine-5(4H)-one;
7-(3-chlorobenzyl)-4-(2,4-difluorobenzyl)-6,7,8,9-tetrahydroimidazo[1,2-a]pyrido[3,4-e]pyrimidine-5(4H)-one;
7-(3-methylbenzyl)-4-(2,4-difluorobenzyl)-6,7,8,9-tetrahydroimidazo[1,2-a]pyrido[3,4-e]pyrimidine-5(4H)-one;
7-(3-cyanobenzyl)-4-(2,4-difluorobenzyl)-6,7,8,9-tetrahydroimidazo[1,2-a]pyrido[3,4-e]pyrimidine-5(4H)-one;
7-benzyl-4-(4-trifluoromethylbenzyl)-6,7,8,9-tetrahydroimidazo[1,2-a]pyrido[3,4-e]pyrimidine-5(4H)-one;
7-(3-fluorobenzyl)-4-(4-trifluoromethylbenzyl)-6,7,8,9-tetrahydroimidazo[1,2-a]pyrido[3,4-e]pyrimidine-5(4H)-one;
7-(3-chlorobenzyl)-4-(4-trifluoromethylbenzyl)-6,7,8,9-tetrahydroimidazo[1,2-a]pyrido[3,4-e]pyrimidine-5(4H)-one;
7-(3-methylbenzyl)-4-(4-trifluoromethylbenzyl)-6,7,8,9-tetrahydroimidazo[1,2-a]pyrido[3,4-e]pyrimidine-5(4H)-one;
7-(3-cyanobenzyl)-4-(4-trifluoromethylbenzyl)-6,7,8,9-tetrahydroimidazo[1,2-a]pyrido[3,4-e]pyrimidine-5(4H)-one;
7-benzyl-4-(4-methylbenzyl)-6,7,8,9-tetrahydroimidazo[1,2-a]pyrido[3,4-e]pyrimidine-5(4H)-one;
7-(3-fluorobenzyl)-4-(4-methylbenzyl)-6,7,8,9-tetrahydroimidazo[1,2-a]pyrido[3,4-e]pyrimidine-5(4H)-one;
7-(3-chlorobenzyl)-4-(4-methylbenzyl)-6,7,8,9-tetrahydroimidazo[1,2-a]pyrido[3,4-e]pyrimidine-5(4H)-one;
7-(3-methylbenzyl)-4-(4-methylbenzyl)-6,7,8,9-tetrahydroimidazo[1,2-a]pyrido[3,4-e]pyrimidine-5(4H)-one;
7-(3-cyanobenzyl)-4-(4-methylbenzyl)-6,7,8,9-tetrahydroimidazo[1,2-a]pyrido[3,4-e]pyrimidine-5(4H)-one;
7-benzyl-4-(4-ethylbenzyl)-6,7,8,9-tetrahydroimidazo[1,2-a]pyrido[3,4-e]pyrimidine-5(4H)-one;
7-(3-fluorobenzyl)-4-(4-ethylbenzyl)-6,7,8,9-tetrahydroimidazo[1,2-a]pyrido[3,4-e]pyrimidine-5(4H)-one;
7-(3-chlorobenzyl)-4-(4-ethylbenzyl)-6,7,8,9-tetrahydroimidazo[1,2-a]pyrido[3,4-e]pyrimidine-5(4H)-one;
7-(3-methylbenzyl)-4-(4-ethylbenzyl)-6,7,8,9-tetrahydroimidazo[1,2-a]pyrido[3,4-e]pyrimidine-5(4H)-one;
7-(3-cyanobenzyl)-4-(4-ethylbenzyl)-6,7,8,9-tetrahydroimidazo[1,2-a]pyrido[3,4-e]pyrimidine-5(4H)-one;
7-benzyl-4-(4-methoxybenzyl)-6,7,8,9-tetrahydroimidazo[1,2-a]pyrido[3,4-e]pyrimidine-5(4H)-one;
7-(3-fluorobenzyl)-4-(4-methoxybenzyl)-6,7,8,9-tetrahydroimidazo[1,2-a]pyrido[3,4-e]pyrimidine-5(4H)-one;
7-(3-chlorobenzyl)-4-(4-methoxybenzyl)-6,7,8,9-tetrahydroimidazo[1,2-a]pyrido[3,4-e]pyrimidine-5(4H)-one;
7-(3-methylbenzyl)-4-(4-methoxybenzyl)-6,7,8,9-tetrahydroimidazo[1,2-a]pyrido[3,4-e]pyrimidine-5(4H)-one;
7-(3-cyanobenzyl)-4-(4-methoxybenzyl)-6,7,8,9-tetrahydroimidazo[1,2-a]pyrido[3,4-e]pyrimidine-5(4H)-one;
7-(3-chlorobenzyl)-4-([1,3]benzodioxol-5-ylmethyl)-6,7,8,9-tetrahydroimidazo[1,2-a]pyrido[3,4-e]pyrimidine-5(4H)-one;
7-(3-chlorobenzyl)-4-((2,3-dihydro[1,4]benzodioxin-6-yl)methyl)-6,7,8,9-tetrahydroimidazo[1,2-a]pyrido[3,4-e]pyrimidine-5(4H)-one;
4-(4-trifluoromethylbenzyl)-7-benzyl-6,7,8,9-tetrahydropyrido[3,4-e][1,2,4]triazolo[1,5-a]pyrimidine-5(4H)-one;
4-(4-trifluoromethylbenzyl)-7-(3-chlorobenzyl)-6,7,8,9-tetrahydropyrido[3,4-e][1,2,4]triazolo[1,5-a]pyrimidine-5(4H)-one;
4-(4-trifluoromethylbenzyl)-7-(3-cyanobenzyl)-6,7,8,9-tetrahydropyrido[3,4-e][1,2,4]triazolo[1,5-a]pyrimidine-5(4H)-one;
4-(4-chlorobenzyl)-7-benzyl-6,7,8,9-tetrahydropyrido[3,4-e][1,2,4]triazolo[1,5-a]pyrimidine-5(4H)-one;
4-(4-chlorobenzyl)-7-(3-fluorobenzyl)-6,7,8,9-tetrahydropyrido[3,4-e][1,2,4]triazolo[1,5-a]pyrimidine-5(4H)-one;
4-(4-chlorobenzyl)-7-(3-chlorobenzyl)-6,7,8,9-tetrahydropyrido[3,4-e][1,2,4]triazolo[1,5-a]pyrimidine-5(4H)-one;
4-(4-chlorobenzyl)-7-(3-cyanobenzyl)-6,7,8,9-tetrahydropyrido[3,4-e][1,2,4]triazolo[1,5-a]pyrimidine-5(4H)-one;
4-benzyl-7-(3-chlorobenzyl)-6,7,8,9-tetrahydropyrazolo[1,5-a]pyrido[3,4-e]pyrimidine-5(4H)-one;
4-benzyl-7-(3-cyanobenzyl)-6,7,8,9-tetrahydropyrazolo[1,5-a]pyrido[3,4-e]pyrimidine-5(4H)-one;
4-benzyl-7-(3-methylbenzyl)-6,7,8,9-tetrahydropyrazolo[1,5-a]pyrido[3,4-e]pyrimidine-5(4H)-one;
4-(4-trifluoromethylbenzyl)-7-benzyl-6,7,8,9-tetrahydropyrazolo[1,5-a]pyrido[3,4-e]pyrimidine-5(4H)-one;
4-(4-trifluoromethylbenzyl)-7-(3-cyanobenzyl)-6,7,8,9-tetrahydropyrazolo[1,5-a]pyrido[3,4-e]pyrimidine-5(4H)-one;
4-(4-chlorobenzyl)-7-benzyl-6,7,8,9-tetrahydropyrazolo[1,5-a]pyrido[3,4-e]pyrimidine-5(4H)-one;
4-(4-chlorobenzyl)-7-(3-chlorobenzyl)-6,7,8,9-tetrahydropyrazolo[1,5-a]pyrido[3,4-e]pyrimidine-5(4H)-one;
4-(4-chlorobenzyl)-7-(3-cyanobenzyl)-6,7,8,9-tetrahydropyrazolo[1,5-a]pyrido[3,4-e]pyrimidine-5(4H)-one;
7-benzyl-4-(4-trifluoromethylbenzyl)-6,7,8,9-tetrahydroimidazo[1,5-a]pyrido[3,4-e]pyrimidine-5(4H)-one;
7-(3-cyanobenzyl)-4-(4-trifluoromethylbenzyl)-6,7,8,9-tetrahydroimidazo[1,5-a]pyrido[3,4-e]pyrimidine-5(4H)-one;
7-(3-chlorobenzyl)-4-(4-chlorobenzyl)-6,7,8,9-tetrahydroimidazo[1,5-a]pyrido[3,4-e]pyrimidine-5(4H)-one;
7-(3-cyanobenzyl)-4-(4-chlorobenzyl)-6,7,8,9-tetrahydroimidazo[1,5-a]pyrido[3,4-e]pyrimidine-5(4H)-one;
3-benzyl-6-(4-trifluoromethylbenzyl)-1,2,3,4-tetrahydropyrido[3,4-e]pyrrolo[1,2-a]pyrimidine-5(6H)-one; and
3-(3-chlorobenzyl)-6-(4-chlorobenzyl)-1,2,3,4-tetrahydropyrido[3,4-e]pyrrolo[1,2-a]pyrimidine-5(6H)-one.

6. A preparation method for the compounds of general formula I according to claim 1, wherein when the ring A is of a structure shown in general formula II, a synthetic route comprises 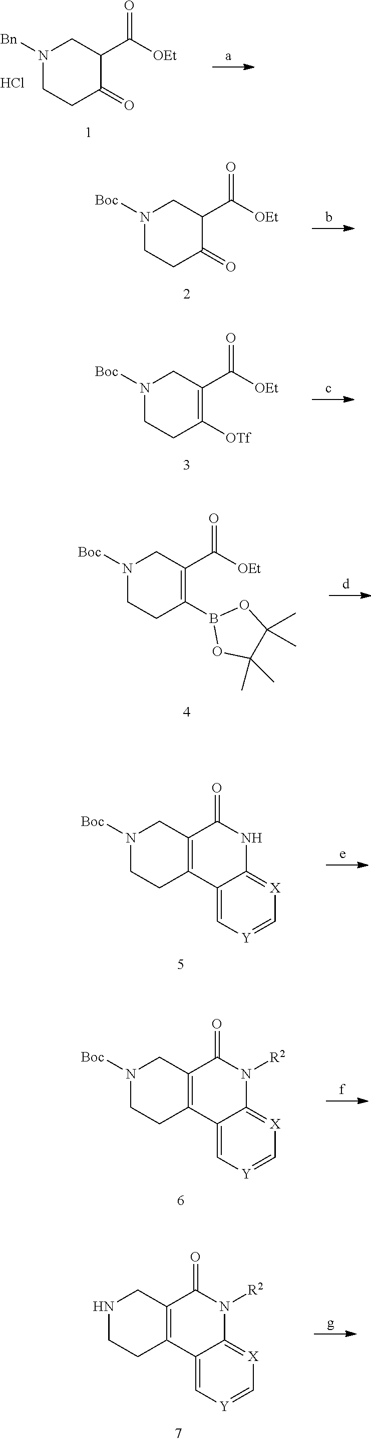 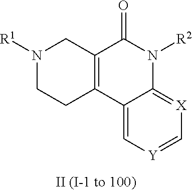
the following steps:
(1) step a: after hydrogenation to remove a benzyl group, reacting compound 1 with di-tert-butyl dicarbonate to prepare compound 2;
(2) step b: reacting compound 2 with trifluoromethanesulfonic anhydride to prepare compound 3;
(3) step c: reacting compound 3 with bis(pinacolato)diboron to prepare compound 4;
(4) step d: reacting the compound 4 with iodo- and amino-disubstituted pyridine, benzene or pyrimidine to prepare compound 5;
(5) step e: reacting the compound 5 with different halogenated hydrocarbons to prepare compound 6;
(6) step f: deprotecting the compound 6 with trifluoroacetic acid from a tert-butoxycarbonyl group to prepare compound 7; and
(7) step g: reacting the compound 7 with different halogenated hydrocarbons to prepare compounds represented by general formula II;
wherein the compound 4 is reacted with other heterocyclic aromatic amines substituted by ortho-iodo or ortho-bromo according to the method described above to synthesize compounds of general formula I in which the ring A is other aromatic heterocyclic rings, including furan, thiophene, and thiazole;
or, when the ring A in general formula I is an imidazole ring of general formula III, a synthetic route comprises 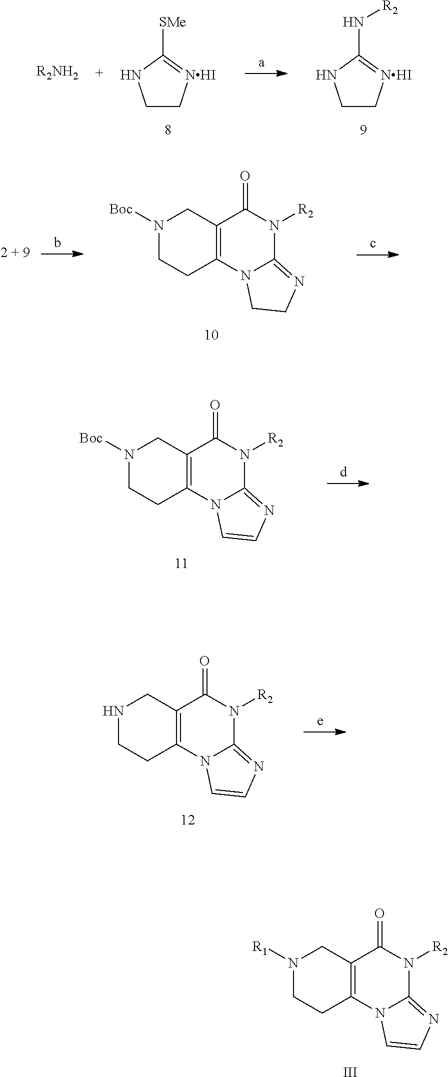
the following steps:
(1) step a: reacting compound 8 with different amines to prepare compound 9;
(2) step b: reacting the compound 2 with the compound 9 to prepare compound 10;
(3) step c: oxidizing a dihydroimidazole ring in the compound 10 into an imidazole ring to prepare compound 11;
(4) step d: deprotecting the compound 11 with trifluoroacetic acid from a tert-butoxycarbonyl group to prepare compound 12; and
(5) step e: reacting the compound 12 with different halogenated hydrocarbons to prepare compounds represented by general formula III;
or, when the ring A in general formula I is an imidazole ring, a triazole ring, a pyrazole ring or a pyrrole ring in general formula IV, and X, Y and Z are each a nitrogen atom or a methine group, a synthetic route comprises: 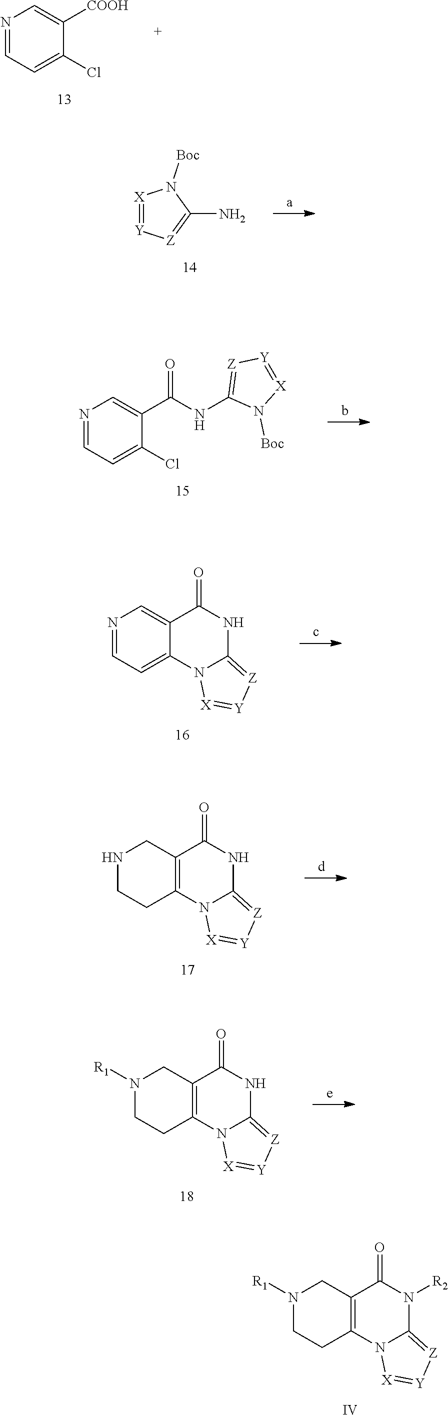
the following steps:
(1) step a: condensing compound 13 with compound 14 to synthesize compound 15; wherein when a five-membered ring of the compound 14 contains multiple nitrogen atoms, a Boc protecting group is on different nitrogen atoms;
(2) step b: removing the Boc protecting group of the compound 15 and then performing cyclization through an intramolecular nucleophilic substitution reaction of aryl groups under basic conditions to prepare compound 16;
(3) step c: reducing a pyridine ring in the compound 16 by hydrogenation to prepare compound 17;
(4) step d: subjecting an amino group in the compound 17 to reductive amination with different aromatic aldehydes to synthesize compound 18;
(5) step e: subjecting the compound 18 to a nucleophilic substitution reaction with different halogenated hydrocarbons to synthesize compounds represented by general formula IV;
or, when the ring A in general formula I is an imidazole ring, a triazole ring or a pyrazole ring in general formula IV, and X, Y and Z are each a nitrogen atom or a methine group, a synthetic route comprises 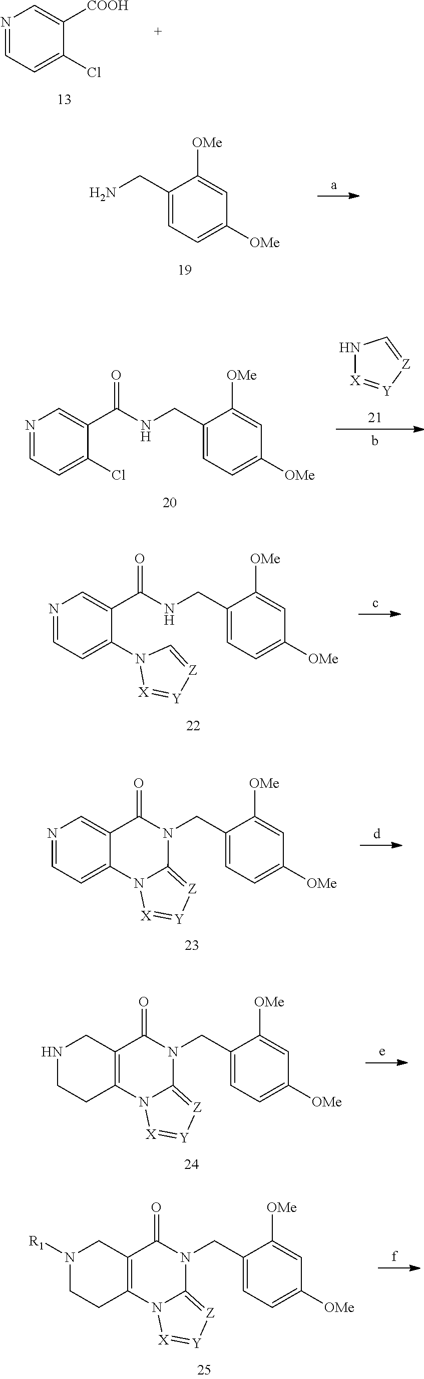 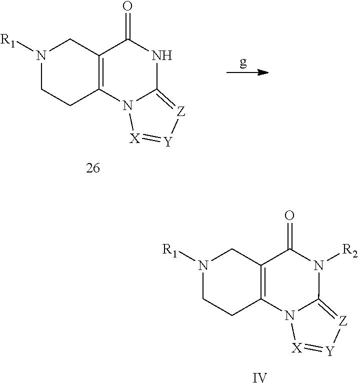
the following steps:
(1) step a: condensing the compound 13 with compound 19 to prepare compound 20;
(2) step b: subjecting the compound 20 to a nucleophilic substitution reaction of aromatic rings with compound 21 under basic conditions to synthesize compound 22;
(3) step c: subjecting the compound 22 to intramolecular coupling and cyclization under the catalysis of a cuprous salt to synthesize compound 23;
(4) step d: reducing pyridine in the compound 23 under catalytic hydrogenation to synthesize compound 24;
(5) step e: subjecting the compound 24 to a nucleophilic substitution reaction with halogenated hydrocarbons to synthesize compound 25;
(6) step f: removing a 2,4-dimethoxybenzyl group of the compound 25 under acidic conditions to prepare compound 26; and
(7) step g: subjecting the compound 26 to a nucleophilic substitution reaction with halogenated hydrocarbons to synthesize compounds represented by general formula IV;
or, a synthetic route for the compounds of general formula IV comprises 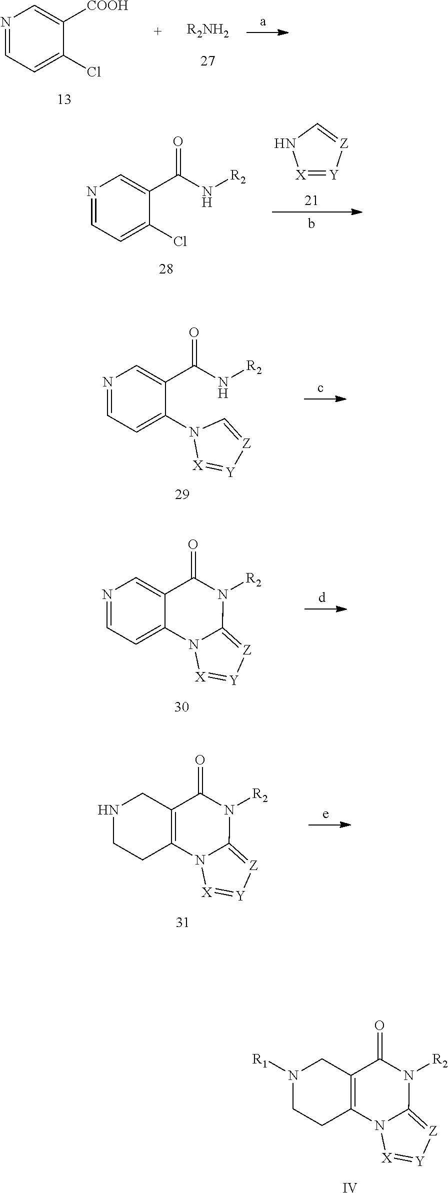
the following steps:
(1) step a: condensing the compound 13 with compound 27 to prepare compound 28;
(2) step b: subjecting the compound 28 to a nucleophilic substitution reaction of aromatic rings with the compound 21 under basic conditions to synthesize compound 29,
(3) step c: subjecting the compound 29 to intramolecular coupling and cyclization under the catalysis of a cuprous salt to synthesize compound 30;
(4) step d: reducing pyridine in the compound 30 under catalytic hydrogenation to synthesize compound 31; and
(5) step e: subjecting compound 31 to a nucleophilic substitution reaction with halogenated hydrocarbons to synthesize compounds represented by general formula IV.

7. Pharmaceutical compositions comprising the compounds or the pharmaceutically acceptable salts thereof according to claim 1, and pharmaceutically acceptable carriers.

8. Use of the compounds according to claim 1 in the preparation of ClpP agonists.

9. Use of the compounds according to claim 1 in the preparation of medicaments for treating cancer.

10. The use according to claim 9, wherein the cancer includes acute myeloid leukemia, breast cancer, lung cancer, liver cancer, ovarian cancer, bladder cancer, prostate cancer, uterine cancer, gastric cancer, testicular cancer, thyroid cancer, cervical cancer, osteosarcoma, neuroblastoma, colon cancer, and brain tumors.

11. Use of the pharmaceutical compositions according to claim 7 in the preparation of ClpP agonists.

12. Use of the pharmaceutical compositions according to claim 7 in the preparation of medicaments for treating cancer.

13. The use according to claim 12, wherein the cancer includes acute myeloid leukemia, breast cancer, lung cancer, liver cancer, ovarian cancer, bladder cancer, prostate cancer, uterine cancer, gastric cancer, testicular cancer, thyroid cancer, cervical cancer, osteosarcoma, neuroblastoma, colon cancer, and brain tumors.
